(19) United States
(12) Patent Application Publication
(10) Pub. No.: US 2023/0040324 A1
(43) Pub. Date: Feb. 9, 2023

(54) AMINOACRIDINE AND AMINOPYRENE DYES AND THEIR USE AS FLUORESCENT TAGS, IN PARTICULAR FOR CARBOHYDRATE ANALYSIS

(71) Applicant: Max-Planck-Gesellschaft zur Foerderung der Wissenschaften e.V., Muenchen (DE)

(72) Inventors: Maksim A. FOMIN, Goettingen (DE); Elizaveta SAVICHEVA, Goettingen (DE); Jan SEIKOWSKI, Goettingen (DE); Vladimir N. BELOV, Goettingen (DE); Stefan W. HELL, Goettingen (DE)

(21) Appl. No.: 17/782,691

(22) PCT Filed: Dec. 6, 2019
(86) PCT No.: PCT/EP2019/084067

§ 371 Date: Jun. 6, 2022

(51) Int. Cl. G01N 33/58; C09B 15/00; C09B 57/00; C09K 11/06; G01N 33/68

(52) U.S. Cl. CPC G01N 33/582; C09B 15/00; C09B 57/001; C09K 11/06; G01N 33/6803; C09K 2211/1018; C09K 2211/1011

(57) ABSTRACT

The invention relates to novel fluorescent dyes with multiple negatively charged groups in their ionized form which are 9-aminoacridines or 1-aminopyrene shaving of one of the following general formulae A-E: or salts or protonated forms thereof, wherein the ionizable groups are typically selected from the following: OH, SH, COOH, SO₃H, OSO₃H, SO₂NHCN, P(O)(OH)₂, P(O) (OH)₂. The invention further relates to the use of these dyes as fluorescent tags, in particular for reducing sugars and glycans, and to carbohydrate-dye conjugates comprising these dyes as well as to methods for preparing the same.

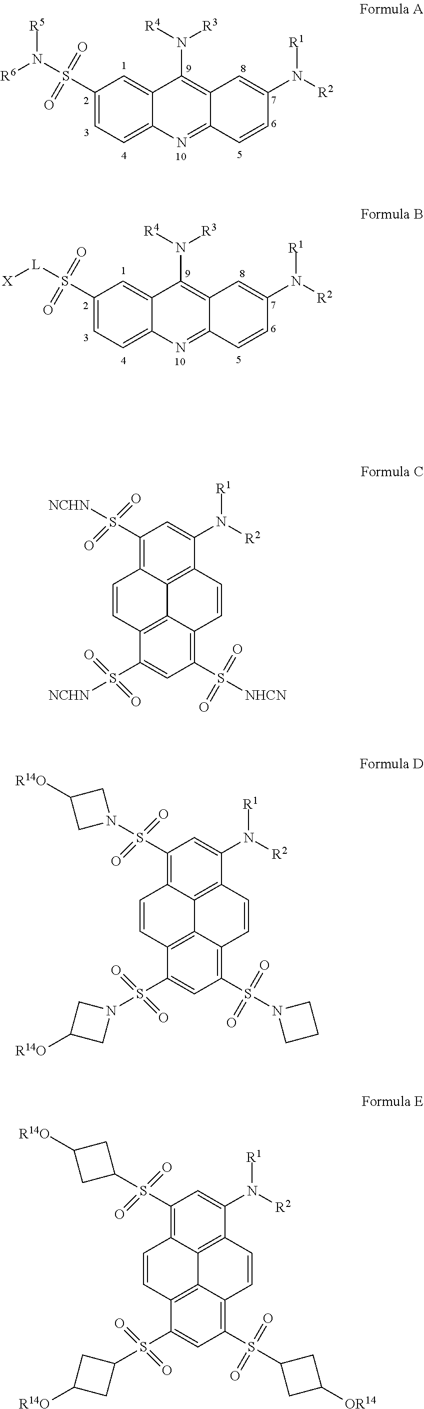

### CROSS-REFERENCE TO RELATED APPLICATIONS

This application is a U.S. National Phase Application of PCT/EP2019/084067, filed Dec. 6, 2019, the contents of which are incorporated herein by reference in their entireties for all purposes.

### FIELD OF THE INVENTION

The present invention provides novel negatively charged fluorescent dyes with aromatic amino groups, low mass-to-charge (m/z) ratios and demonstrates their use as fluorescent tags facilitating separation of the analytes and their sensitive detection by fluorescence. The novel dyes contain polycyclic aromatic systems (acridine, pyrene), anionic groups (e.g., primary phosphate, N-cyanosulfonamide) and are applied within carbohydrate analysis, in particular, as reagents for reductive amination of glycans, followed by separation and fluorescence detection of the carbohydrate derivatives. Their emission wavelengths are finely tunable over a wide range including green and red regions. The separation techniques applicable for the dyes of the present invention (e.g., hydrophilic interaction chromatography (HILIC), reversed phase liquid chromatography (HPLC), electrophoresis, in particular, capillary (gel) electrophoresis with laser-induced fluorescence detection) can be coupled to mass-spectrometry, which can be used alone (as a detection method) or in addition to fluorescence detection.

### BACKGROUND OF THE INVENTION

Glycosylation reactions are defined as enzymatically driven and highly diverse transformations of proteins, lipids or other noncarbohydrates in eukaryotic cells. Glycosylation creates a new chemical bond between a carbohydrate (the glycone; donor) and another (noncarbohydrate) molecule (the aglycon; acceptor). The term “carbohydrate(s)” as used herein is the collective term for monosaccharide(s), like xylose arabinose, glucose, galactose, mannose, fructose, fucose, N-acetylglucosamine, homo or hetero sialic acids; disaccharide(s), like lactose, sucrose, maltose, cellobiose; glycans (e.g. N- and O-glycans), homo- or hetero oligosaccharide(s), galactooligosaccharides (GOS), fructooligosaccharides (FOS), milk oligosaccharides (MOS), or even glycomoieties of glycolipids; and, in particular, polysaccharide(s), like amylose, amylopektin, cellulose, glycogen, glycosaminoglycan, or chitin. Oligo- and polysaccharides can be linear, branched or multiply branched. Typically, the aglycone is either a protein (“glycoprotein”) or a lipid (“glycolipid”). In a more general sense, “glycoconjugate” means a carbohydrate covalently linked to any other chemical entity such as protein, peptide, lipid, or even saccharide.

Glycoconjugates represent the most structurally and functionally diverse entities in nature (FIG. 5) and are involved in protein folding and stability, regulation of protein bioactivity, cell-cell recognition, protease resistance, cellular signaling, regulation and developmental processes, interactions with hormones, toxins and antibodies. However, only few specific functions of these complex and carbohydrate-rich molecules have been well understood so far.

Glycoconjugates range in complexity from the relatively simple glycolipids to extraordinarily complex and multiple glycosylated proteins. Although the carbohydrate moiety is commonly composed of only a few monosaccharides, including N-acetylglucosamine, N-acetylgalactosamine, mannose, galactose, fucose, glucose, and sialic acids, their structural diversity (not yet fully revealed) is much larger than that of proteins or DNA. The reasons for this diversity are the presence of the anomers and the ability of monosaccharides to branch and build different (glycosidic) linkages. Accordingly, an oligosaccharide with a relatively small chain length may have an enormous number of structural isomers. Moreover, unlike protein biosynthesis, which is based on RNA as a template, the information flow from the genome to the glycome is a complex and no longer a template-driven process. The co- and post-translational modifications of proteins in glycan biosynthesis are caused by enzymatic reactions. This leads to a drastic increase of complexity and structural diversity of glycans (as depicted in FIG. 5). In practice, the term glycan refers to the carbohydrate portion of a glycoconjugate. The terms glycan, oligosaccharide and polysaccharide are synonyms and mean “compounds consisting of a medium to large number of monosaccharides linked glycosidically” (a IUPAC definition).

In order to elucidate the structural features of glycome (the complete set of glycoconjugates that cells produce under specified conditions) and to understand its functions and interaction with the DNA and protein “machinery”, rapid, robust and high resolution bioanalytic techniques must be available.

Many analytic techniques have been utilized to analyze carbohydrates. Complex samples containing a variety of different oligosaccharides can be separated by chromatographic or electrokinetic techniques. The chromatographic techniques include size exclusion chromatography (SEC), hydrophilic interaction chromatography (HILIC), high-performance liquid chromatography (HPLC), reversed-phase ion-pairing chromatography (RPIPC), and porous graphitized carbon chromatography (PGC). Mass spectrometry (MS) and nuclear magnetic resonance (NMR) allow de novo structural characterization of complex oligosaccharides. A combination of several techniques is often applied, e.g. HPLC-NMR, HPLC-MS (also called LC-MS; LC=liquid chromatography) or capillary electrophoresis coupled with mass-spectrometry (CE-MS). As soon as the structure of a particular glycan is determined, its characteristic properties, such as constitution, net charge, the exact molecular mass, retention or migration times, are tabulated. By comparing the data obtained for unknown samples with tabulated parameters, the rapid screening and evaluation of unknown samples can be performed.

Carbohydrates do not absorb visible light; for the sensitive fluorescence detection they must be labeled with fluorescent tags. Thus, for analysis via chromatographic or electrokinetic separation followed by fluorescence detection, it is necessary to label the oligosaccharides with fluorescent dyes prior to the analysis. Consequently, besides providing proper excitation and emission wavelengths (and additional electric charges required for electrophoretic separation; see below), these dyes must have a reactive group.

The most common and straightforward method for labeling carbohydrates with fluorescent dyes is the reductive amination (FIG. 6). The two-step procedure (see next section for details) yields a covalent bond between the reducing end of a carbohydrate (acetal or aldehyde group) and a primary amine (see, for example, N. Volpi, Capillary electrophoresis of carbohydrates. From monosaccharides to complex polysaccharides, Humana Press, New York, 2011, pp. 1-51). The primary amino group is either incorporated into the structure of the fluorophore or bound to a linker, which is connected with the fluorescent dye.

The electrokinetic separation is based on the motion of charged particles under the influence of an applied electric field and includes various electrophoretic techniques. The most important electrokinetic separation methods for glycoanalysis are capillary zone electrophoresis (CZE) and capillary (gel) electrophoresis (C(G)E). These techniques provide high resolution, fast separation, and allow for quantification. Capillary electrophoresis (CE) is a family of electrokinetic separation methods defined by the IUPAC as “separation techniques carried out in capillaries based solely on the differences in the electrophoretic mobilities of charged species (analytes) either in aqueous or non-aqueous background electrolyte solution” (see also M.-L. Riekkola, J. A. Jönsson, R. M. Smith, Pure Appl. Chem. 2004, 76, 443-451). In other words, CE terms the separation of ions by electrophoresis within a thin capillary. CGE is defined by the IUPAC as “a special case of capillary sieving electrophoresis when the capillary is filled with a cross-linked gel” (see also M.-L. Riekkola et al., above). The net electrical charge is required for separation of the analytes by C(G)E. The electrophoretic mobility of a compound depends on the mass to charge ratio, and when employing CGE—due to the gels sieving effect—it depends additionally on the molecular shape. The native carbohydrates, except sialic or glucuronic acids, sulphated or phosphorylated derivatives, are electroneutral and cannot be separated by their mass to charge ratio (electrophoresis). Since the size of a molecule is strongly related to the mass, and the size is rather difficult to evaluate, it is reasonable to use the mass to charge ratio as an approximation for predicting the electrophoretic mobility.

Remarkably, the mass to charge (or size to charge) ratio of many macromolecules such as DNA is virtually constant because each nucleotide adds practically the same mass and charge to the macromolecule keeping the mass to charge ratio nearly constant on average [D. N. Heiger, High performance capillary electrophoresis. An introduction: a primer, Agilent Technologies, Germany, 2000, pp. 19-24, 70]. This regularity may limit the separation ability of electrophoresis. However, the limitation can be overcome by using capillary gel electrophoresis. The capillary is filled with gel, and the separation works like in traditional gel electrophoresis, but using 10 to 100 times higher electric fields without the negative effects of Joule heating [due to efficient heat dissipation; H. H. Lauer, G. P. Rozing, High Performance Capillary Electrophoresis. A Primer. Agilent Technologies, 2009, pp. 17, 60-65, 157-159]. Since small molecules are likely to pass more pores than large molecules, small molecules move faster through the gel matrix. Due to this sieving effect, not only the mass to charge ratio but also the size and shape of a sample ion are affecting the electrophoretic mobility. The factor of molecular size is important, as it enables to use DNA-ladder (mixture of DNA oligomers of various lengths labeled with a fluorescent dye) as an internal standards for calibration of the migration times (see below). The factors of molecular size and shapes predict that the fluorescent tags providing fast-moving derivatives of glycans must have compact structures (one of the aims of the present application).

Capillary gel electrophoresis (CGE) emerged as a particularly important method for analyzing glycoconjugates including glycoproteins, glycopeptides and “released” (enzymatically cleaved from the acceptors) N- or O-glycans ((a) Novotny, M. V.; Alley, W. R., Recent trends in analytical and structural glycobiology. Curr. Opin. Chem. Biol. 2013, 17 (5), 832-840. (b) Lu, G.; Crihfield, C. L.; Gattu, S.; Veltri, L. M.; Holland, L. A. Chem. Rev. 2018, 118, 7867-7885. (c) Ruhaak, L. R.; Xu, G.; Li, Q.; Goonatilleke, E.; Lebrilla, C. B. Chem. Rev. 2018, 118, 7886-7930.) Combined with laser induced fluorescence detection (LIF), this method (CGE-LIF) allows fast and very fine resolution of analytes according to their charge-to-size ratio (G. Lu, C. L. Crihfield, S. Gattu, L. M. Veltri, L. A. Holland, Chem. Rev. 2018, 118, 7867-7885). Since the hydrodynamic radius is difficult to evaluate, the mass-to-charge ratio (m/z) is used as an estimate of the electrophoretic mobility. The main reason for using CGE-LIF is the sensitivity of detection provided by fluorescence (measuring the emission intensity) and the possibility to improve separation of the analytes by labeling them with fluorescent markers having additional electrical charges.

The procedure of glycan analysis can be divided into the following steps: sample preparation (e.g. deglycosylation), glycan labeling, sample purification, chromatographic or electrokinetic separation with detection by emission and the data analysis. The present invention introduces the negatively charged organic dyes with an amino group for glycan labeling, followed by electrophoretic separation of the derivatives obtained upon labeling and, finally, their detection by fluorescence (with an additional option of quantification). In another embodiment, the present invention introduces the negatively charged and red-emitting organic dyes with an amino group for reductive amination of carbohydrates and preparation of internal standards—mixtures of oligosaccharides labeled with a fluorescent dye detectable independently from the analytes (e.g., in a separate “color channel” of a fluorescence detector). The internal standards' mixture is injected together with an analytical sample. The components of an internal standard must have retention times covering the whole range of the analytically relevant compounds. This aspect of the present application will be discussed in detail below.

Importantly, the features of an “ideal” fluorescent tag intended for modification of glycans followed by the electrophoretic separation and detection of the derivatives—the reactive group, electrical charge and emissive properties—can be incorporated into one structure with (multiple) charges and the amino group reacting with aldehyde residues in reducing sugars. After derivatization (reductive amination) and purification (to remove proteins, excess electrolytes, excess dye, labeling reagents, etc.), the labeled sample is injected into the chromatographic column, or the electrokinetic capillary, and the separation is carried out. Due to different properties (structure, polarity, mass/charge ratio, shape, etc.), the carbohydrates are separated and reach the detector according to their characteristic retention, or migration times. When the labeled carbohydrates reach the fluorescence detector, the fluorescent markers are excited (most often with 488 nm (argon) laser or 505 nm solid state laser), and the emission signal is detected.

The first step of the reductive amination (FIG. 6) involves the nucleophilic attack of an amino group to an aldehyde group of the carbohydrate residue in its open-chain form (1). The acid-catalyzed elimination of water from intermediate 2 (or its protonated equivalent) gives imine 3. The imine formation is reversible, but imine 3 can be converted into a secondary amine (5) via irreversible acid-catalyzed reduction of iminium salt 4 (formed from compound 3 upon protonation) with a borohydride, such as sodium cyanoborohydride, triacetoxy borohydride, 2-picoline-borane, or similar reagents. The nature of borane is important, because only iminium ions 4 must be reduced, while carbohydrates R²CHO (1) have to remain unreactive towards the reduction. Additionally, the borane reagent has to be sufficiently stable under acidic conditions.

Thus, the applicability of the reaction sequence depicted in FIG. 6 to carbohydrate analysis is based (besides fluorescent dyes R³NH₂) on the availability, stability and sufficient reactivity of special reducing agents (boranes) which do not react with aldehydes (or reduce them very slowly), but under acidic conditions readily reduce iminium ions (4). Most often, commercially available sodium cyanoborohydride and 2-picoline borane are used for reductive amination of glycans. Weak or medium strong acids such as acetic (pKₐ=4.76), malonic (pK¹ₐ=2.83) or citric acid (pKia=3.13) are frequently used at pH=3-6 to achieve an irreversible and rapid reduction (K. R. Anumula, Anal. Biochem. 2006, 350, 1-23).

Therefore, the applied amine (fluorescent dye R³NH₂) has to be a weak base, because the full protonation of amine R³NH₂ in FIG. 6 slows down or even blocks its reaction with aldehyde 1. An example of such a base is aniline with pKₐ value of about 4.5 (this is the value for the conjugated acid), which have been used to detect reducing sugars on a TLC plate. In proteins, the aliphatic amino groups of lysine, nucleophilic nitrogen atoms in histidine and arginine residues are mostly protonated at pH=2-6, which disfavors their reaction according to FIG. 6. Aromatic amines with rather low pKₐ values of 2-5 are required and widely used as analytical reagents for reductive amination of natural glycans. Shown below are four commercially available aromatic amines applicable for labeling of glycans via reductive amination followed by electrokinetic separation of conjugates and detection by fluorescence.

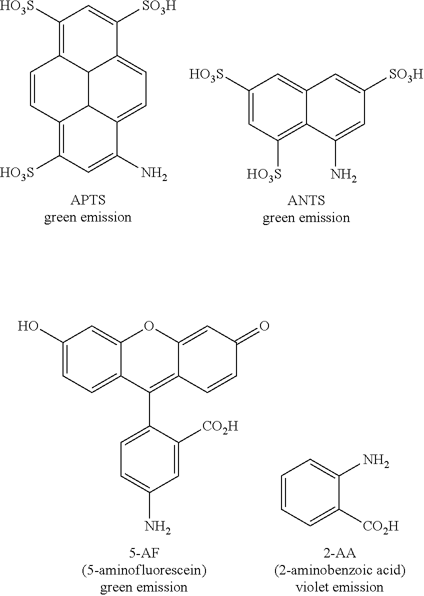 [SMILES: NC1=CC(S(=O)(=O)O)=C2C=CC3=C(S(=O)(=O)O)C=C(S(=O)(=O)O)C4=CC=C1C2C43 ; Nc1cc(S(=O)(=O)O)cc2cc(S(=O)(=O)O)cc(S(=O)(=O)O)c12 ; Nc1ccc(-c2c3ccc(=O)cc-3oc3cc(O)ccc23)c(C(=O)O)c1 ; Nc1ccccc1C(=O)O]

The aromatic amines are negatively charged (or potentially charged) groups. These compounds can be used for reductive amination of carbohydrates. Emission colors of conjugates (obtained according FIG. 6) are given. For details, see: N. V. Shilova, N. V. Bovin, Russ. J. Bioorg. Chem. 2003, 29 (4), 339-355. 3-Aminopyrene-1,6,8-trisulfonic acid (APTS) is a bench-mark dye which is widely used as a reagent for carbohydrate labeling. It is by far the best available marker for glycan analysis in respect of spectral properties and electrophoretic mobility. Three strongly acidic residues (sulfonic acid groups) introduce three negative charges at pH>2 (in a very wide pH range). Conjugates of APTS dye can be efficiently excited with an argon laser (488 nm and 514 nm lines with intensity ratio of 3:1) which is a part of many commercially available DNA sequencing equipment applied for glycan analysis by means of CGE-LIF. ANTS dye also has three negative charges, but its absorption maximum is blue-shifted (350 nm in conjugates) and, therefore, N-alkyl derivatives of ANTS could not be efficiently excited with a 488 nm laser. 5-Aminofluorescein (5-AF) has a drawback; it may be reduced by boranes into the colorless leuco-form (as a free dye and in conjugates), which has to be re-oxidized. This additional chemical step is undesirable, as it is often accompanied by the formation of by-products interfering with compounds present in the analytical sample. Conjugates of 2-aminobenzoic acid (2-AA) emit violet light (420 nm) and cannot be excited with 488 nm laser. A high throughput analysis of fluorescent glycan derivatives is performed on commercial multiplex CGE-systems, e.g., DNA sequencers equipped with a CGE-LIF module (W. Laroy, R. Contreras, N. Callewaert, Nat. Protoc. 2006, 1, 397-405.). These instruments contain a multiplexed capillary gel electrophoresis unit with laser-induced fluorescence (xCGE-LIF) for the separation of charged analytes (e.g., APTS-labeled glycans), an excitation laser (e.g., a 488 nm laser, argon laser or a 505 nm solid state laser) and a fluorescence detector.

APTS is a routinely used fluorescent marker for labelling of glycans (R. A. Evangelista, M.-S. Liu, F.-T. A. Chen, Anal. Chem. 1995, 67, 2239-2245.). The main spectral properties of APTS and its conjugates with glycans are given in FIGS. 7 and 8. The “brightness” of a fluorescent dye as a glycan label is termed as a product of the extinction coefficient (at 488 nm, an excitation wavelength) and the fluorescence quantum yield. The fluorescence quantum yields of APTS conjugates (R=alkyl; FIG. 7) are always high (>0.8). For APTS conjugates, the extinction coefficient at the maximum (457 nm) is 17000, and the absorption at 488 nm is ca. 35% of the maximal intensity (FIG. 8; Evangelista, R. A.; Liu, M.-S.; Chen, F.-T. A. Anal. Chem. 1995, 67, 2239-2245).

1-Aminopyrene dyes other than APTS have been disclosed as fluorescent tags for carbohydrates. WO 2012/027717 A1 describes systems comprising functionally substituted 1,6,8-trisulfonamido-3-aminopyrenes (APTS derivatives), an analyte-reactive group, a cleavable anchor as well as a porous solid phase. WO 2010/116142 A2 describes a large variety of fluorophores and fluorescent sensor compounds which also encompass aminopyrene-based dyes. However, these documents do not disclose negatively charged 1,6,8-tris-[(3-hydroxy-azetidinyl)sulfonyl]-3-aminopyrenes or 1,6,8-tris-[(3-hydroxycyclobutyl)sulfonyl]-3-aminopyrenes according to the present invention.

Previously, the use of 7-aminoacridone-2-sulfonamides with multiple negatively charged groups and orange emission (for example VBDP in FIG. 10) as fluorescent tags for carbohydrate analysis was described (PCT/EP2019/051396). Acridone dyes structurally related to 9-aminoacridines of the present application are described in WO 2002/099424 A3 and WO 2009/112791 A1. However, these documents do not disclose either 7-aminoacridone-2-sulfonamides from PCT/EP2019/051396 or 7,9-diaminoacridin-2-sulfonamides according to the present application.

U.S. Pat. No. 9,127,164B2 (Fluorescent dyes and uses thereof) describes 9-aminoacridine dyes and peptides obtained from them. This document anticipates 9-aminoacridines independently substituted with amino or alkylamino groups, and electron-withdrawing groups such as halogen, amide, cyano, nitro, carbonyl, carboxyl, sulphonic acid etc. WO 2013/093481 A1 (Fluorescent dyes based on acridine and acridinium derivatives) describes acridine dyes and peptides obtained from them. This document anticipates acridines independently substituted with amino or alkylamino groups, and electron-withdrawing groups such as halogen, amide, cyano, nitro, carbonyl, carboxyl, sulphonic acid etc. US20120220537A1 (9-Aminoacridine derivatives, their preparation and uses) describes the synthesis of N-substituted 9-aminoacridines with electron-withdrawing groups or electron-donating groups. WO2011127406A2 (Acridines as inhibitors of haspin and dyrk kinases) describes N-substituted 9-aminoacridines as inhibitors of haspin and dyrk kinases. This document anticipates acridines independently substituted in 2-, 6- and 7-positions with —CN, —C(O)NR^{A}R^{B}, —S(O)₂R^{A}, —S(O)₂NR^{A}R^{B}, or —NR^{A}R^{B} etc.

Though the documents mentioned above represent the closest prior art, they are irrelevant to the subject matter of the present application, because here are claimed specific compounds with multiple negative charges localized on primary phosphate (—OP(O)(OH)₂ with two negative charges at pH 8 and above) or N-cyanosulfonamide (—SO₂NHCN with one negative charge at pH>5-6) groups which are covered by the general formula C.

None of these dyes has been previously shown or suggested to have superior spectral and electrophoretic properties, in particular as conjugates with carbohydrates, in comparison with APTS. APTS is a unique dye for reductive amination of oligosaccharides (Pabst, M.; Kolarich D.; Poltl, G.; Dalik, T.; Lubec, G.; Hofinger, A.; Altmann, F. Anal. Biochem. 2009, 384, 263-273), but its performance as a fluorescent tag providing only one emission color, moderate brightness and three negative charges, is limited. The fluorescence of APTS labeled glycans is captured in the “green” color channel of the standard LIF detectors of DNA sequencers. APTS and its structural analogs—Teal™ and Turquoise™ dyes—are excitable with an argon laser (emission lines 488 nm and 514 nm) (H.-T. Feng, P. Li, G. Rui, J. Stray, S. Khan, S.-M. Chen, S. F. Y Li, Electrophoresis 2017, 38, 1788-1799). In this context, “the superior spectral and electrophoretic properties” would mean higher brightness (product of the absorbance at the excitation wavelength and fluorescence quantum yield), red-shifted emission, and higher electrophoretic mobility (to reveal “heavy” and slowly moving glycans, yet undetectable as APTS conjugates). The higher brightness is required not only upon excitation with 488 nm light (or Argon laser), but also with 505 nm light (solid state laser in new DNA sequencers).

The red-shifted emission of the new dyes would mean fluorescent tags having minimal interference with the APTS detection window (and different migration profile due to different structure and net electrical charge). The new dyes are needed to cross-validate or increase the precision of glycan identification. The use of a “second dye” complementary to APTS is expected to provide different selectivity profile for complex mixtures of carbohydrates. The new set of migration times based on a new fluorescent tag will enable to create new databases for glycan identification.

Labelled glycans are identified by comparison of their migration times with a standard, the so-called “LIZ 600 DNA Ladder”, which is added to the glycan sample (H.-T. Feng, P. Li, G. Rui, J. Stray, S. Khan, S.-M. Chen, S. F. Y Li, Electrophoresis 2017, 38, 1788-1799). The standard consists of several (ten to twenty) DNA oligomers labeled with a red-emitting fluorescent dye of unknown structure (FRET pair of dyes with a donor absorbing at 488 nm, and an acceptor emitting at about 600 nm). The oligomers have variable migration times covering the region where all “analytically important” APTS conjugates appear in the course of CGE-LIF. The standard DNA Ladder is detected in the red region of the emission spectrum, whereas the labeled glycan samples are detected in the green region. Both samples—mixture of labeled glycans and LIZ 600 DNA Ladder—are excited by the same laser source, thus enabling their simultaneous detection within the same run (without any cross-talk in the detection channel). However, the DNA-based standard is not an ideal marker (US20170369431A1, EP2112506 A1). The structures and shapes of DNA molecules are very different from the structures and shapes of natural glycans. Therefore, the migration parameters of the DNA-based standards “drift” (change in time) differently than these of APTS-labeled glycans. In this context, “drift” means the (long-term) changes of the migration times caused by various factors, e.g., ageing of the gel in capillaries, fluctuations in temperature, buffer concentration, etc. Therefore, up to now, the usability and reproducibility of CGE-LIF for glycan analysis is compromised (limited) by improper and imprecise alignment of migration times of the analytes. The peak of each analyte is positioned between two peaks of the standard, and if the positions of these three peaks drift not uniformly, identification becomes difficult. New fluorescent dyes capable of reductive amination are highly needed to create better internal standards based on glycan oligomers of various lengths decorated with a fluorescent tag (a red-emitting and negatively charged dye detected separately from APTS-glycan conjugates). Maltodextrin oligomers are commercially available and represent the so-called “dextran ladder” consisting of glucose (monomer, M=180), maltose (dimer, M=342), maltotriose (trimer, M=504), etc. For an “n-mer”, the molecular mass will be 180n-18(n−1)=162n+18. Dextran oligomers with average molecular masses of 1000, 5000 (ca. “31-mer”), 9000-11000 and 12000 Da are available from Sigma-Aldrich (Merck). These oligosaccharides can be labeled with APTS dye and provide the so-called “APTS-dextran ladder”. Analogously, labeling of a “dextran ladder” with another (red-emitting and negatively charged) dye will result in a new “dye-dextran ladder” usable as an internal standard for calibration of the migration times. The main challenge is to design and prepare a compact fluorescent dye (with an aromatic amino group and multiple negative charges) excitable with 488 nm or 505 nm light and emitting red light (ca. 600 nm), far away from the band-edge emission of APTS conjugates (520 nm). This would mean the dye with a large Stokes shift (separation between the absorption and emission maxima) of ca. 100 nm. These dyes are rare and have low quantum yields.

In view of the drawbacks of the fluorescent dyes of the prior art, the main objective of the present invention was to provide novel fluorescent dyes with improved properties, such as even higher electrophoretic mobility and/or higher brightness or other favorable spectroscopic characteristics, as compared to APTS. These properties are highly demanded from fluorescent tags for carbohydrate analysis based on electrokinetic separation with fluorescence detection.

This objective has been achieved according to the present invention by providing novel 9-aminoacridine and 1-aminopyrene dyes with multiple negatively charged groups according to claims 1-10, as well as by providing the carbohydrate-dye conjugates comprising these dyes according to claim 14-15. Further aspects and more specific embodiments of the invention are the subject of further claims.

### DESCRIPTION OF THE INVENTION

The structures of the novel fluorescent dyes of the invention are selected from the group consisting of compounds of the following general Formulae A-E or protonated forms or salts thereof:

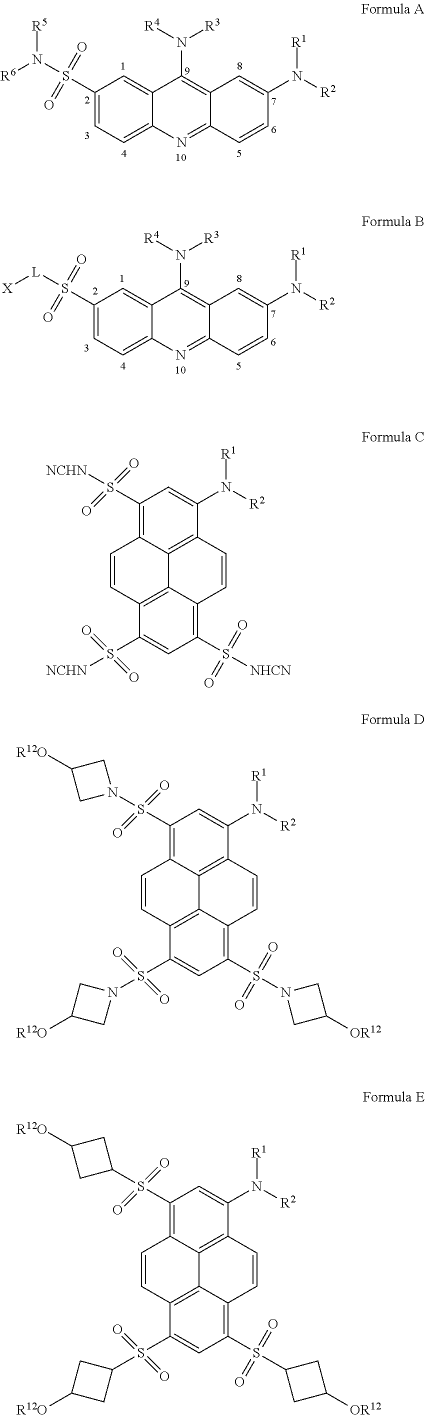

Specifically, the structures of the novel 9-aminoacridine fluorescent dyes with an additional amino group (at C-7) are selected from compounds having the following Formulae A (7,9-diaminoacridine-2-sulfonamides), B (7,9-diaminoacridine-2-sulfones) or salts (protonated forms) thereof:

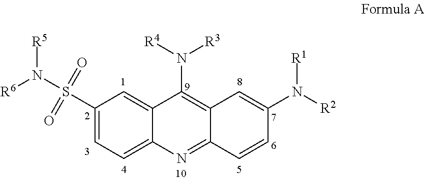

wherein
R¹, R², R³, R⁴, R⁵, R⁶ are independent from each other and selected from the group consisting of
(a) hydrogen;
(b) CH₃, C₂H₅, a straight or branched C₃-C₆, preferably C₃-C₄, alkyl, deuterated alkyl, or perfluoroalkyl group;
(c) straight or branched alkyl group (C₂-C₆) that is unsubstituted, substituted with 1 to 3 halogen atoms (in particular F, Cl, Br), or incorporates 1 to 3 heteroatoms (in particular O, S, N), or optionally substituted with solubilizing groups (in particular OH, NH₂, SH, COOH) and/or ionizable anionic residues, in particular consisting of or including a moiety selected from the group comprising (alkyl) carboxy (CH₂)ₙCO₂H, (alkyl) sulfonate (CH₂)ₙSO₃H, (alkyl) sulfate (CH₂)ₙOSO₃H, (alkyl) phosphate (CH₂)ₙOP(O)(OH)₂ or (alkyl) phosphonate (CH₂)ₙP(O)(OH)₂, wherein n is an integer ranging from 0 (zero) to 6, or analogs thereof wherein one or more of the CH₂ groups are replaced by CF₂ and/or CD₂;
(d) (CH₂)ₙCOOR⁷, where n=1-6, preferably 1-4, and R⁷ may be alkyl, in particular C₁-C₆ alkyl, CH₂CN, CH₂CF₃, benzyl, substituted benzyl, polyhaloalkyl, polyhalophenyl, e.g. tetra- or pentafluorophenyl, pentachlorophenyl, 2- and 4-nitrophenyl, N-succinimidyl, sulfo-N-succinimidyl, 1-benzotriazolyl, 9-aza-1-benzotriazolyl or other groups forming an “active” ester of a carboxylic acid;
(e) a carbonate or carbamate derivative (CH₂)ₘOCOOR⁸ or (CH₂)ₘNHCOOR⁸, where m=1-6 and R⁸=lower alkyl (C₁-C₄), benzyl, 9-fluorenylmethyl, CH₂CN, N-succinimidyl, sulfo-N-succinimidyl, 1-benzotriazolyl, phenyl, substituted phenyl group, e.g., 2- or 4-nitrophenyl, pentachlorophenyl, pentafluorophenyl, 2,3,5,6-tetrafluorophenyl, 2-pyridyl, 4-pyridyl, pyrimid-4-yl;
(f) (CH₂)ₘNR^{a}R^{b}, where m=1-6, preferably 2-4, with a straight or branched alkyl chain; R^{a}, R^{b} are independent from each other and represent hydrogen, C₁-C₄ alkyl and/or C₁-C₄ acyl groups, optionally substituted with a hydroxyalkyl group (CH₂)ₙOH, where n=1-6, with a straight or branched alkyl chain, a phosphorylated hydroxyalkyl group (CH₂)ₚOP(O)(OH)₂, where p=1-6, with a straight or branched alkyl chain;
(g) an alkyl azide (CH₂)_{q}N₃, where q=1-8, preferably 2-6, with a straight or branched alkyl chain;
(h) an alkylalkyne (CH₂)ᵣC<triple bond>CR⁹, where r=1-8, with a straight or branched alkyl chain in (CH₂)ᵣ and R⁹=hydrogen, alkyl C₁-C₆ or (functionally) substituted alkyl C₁-C₆;
(i) (CH₂)ₛCONHR¹⁰, with s=1-8, preferably 1-5; R¹⁰═H, C₁-C₆ alkyl, (CH₂)_{q}N₃, (CH₂)ᵣC<triple bond>CH or (CH₂)ₜ—N-maleimido, (CH₂)ᵤ—NH—COCH₂X (X═Br or I), where q, r, t, u indices are independent in the range 1-8, preferably 2-6, and with straight or branched alkyl chains in (CH₂)ₛ, (CH₂)_{q}, (CH₂)ᵣ, (CH₂)ₜ, (CH₂)ᵤ and R¹⁰;
(j) a primary amino group (NH₂) or secondary amino group (NHR¹¹); R¹¹=alkyl C₁-C₆ or (functionally) substituted alkyl C₁-C₆;
R¹, R², R³, R⁴, R⁵, R⁶ may contain a terminal alkyloxyamino group (CH₂)ᵥONH₂, where v=1-12, preferably 2-6, with a straight or branched alkyl chain that can include one or multiple (alkyl)oxy (CH₂)ₖO, (alkyl)amino (CH₂)ₗNH or (alkyl)amido (CH₂)_{w}CONH groups in all possible combinations with indices k, l and w=0-12 (independent from each other);
R¹, R², R³, R⁴, R^{5′} R⁶ may contain a terminal acylhydrazino group (CH₂)ₓCONHNH₂, where x=1-12, preferably 2-6, with a straight or branched alkyl chain that can include one or multiple (alkyl)oxy (CH₂)ₖO, (alkyl)amino (CH₂)ₗNH or (alkyl)amido (CH₂)_{w}CONH groups in all possible combinations with indices k, l and w=0-12 (independent from each other);
one of the residues R¹, R², R³, R⁴ may represent C(O)R¹² or COOR¹³, where R¹² or R¹³ ═C₁-C₄ alkyl, optionally substituted with a hydroxyalkyl group (CH₂)_{y}OH, where y=1-6, with a straight or branched alkyl chain, a phosphorylated hydroxyalkyl group (CH₂)ₘOP(O)(OH)₂, where m=1-6, with a straight or branched alkyl chain;
one of the residues R⁵, R⁶ may represent CN;
further, one of the residues R¹, R², R³, R⁴, R⁵, R⁶ may represent (CH₂)_{z}—C₆H₄—NH₂, COC₆H₄—NH₂, CONHC₆H₄—NH₂ or CSNHC₆H₄—NH₂ with z=1-4, and C₆H₄ being a 1,2-, 1,3- or 1,4-phenylene, COC₅H₃N—NH₂ or (CH₂)ₛ—C₅H₃N—NH₂, with z=1-4, and C₅H₃N being pyridin-2,4-diyl, pyridin-2,5-diyl, pyridin-2,6-diyl, or pyridin-3,5-diyl; additionally, R¹-R² (R³-R⁴, R⁵-R⁶) may form a four-, five, six-, or seven-membered cycle (comprising the nitrogen atom); which heterocycle may be unsubstituted or substituted with a straight or branched alkyl group (C₂-C₆), which is unsubstituted, substituted with 1 to 3 halogen atoms (in particular F, Cl, Br) or incorporates 1 to 3 heteroatoms (in particular O, S, N), or optionally substituted with solubilizing groups (in particular OH, NH₂, SH, COOH) and/or ionizable anionic residues, in particular consisting of or including a moiety selected from the following groups: (alkyl) carboxy (CH₂)ₙCO₂H, (alkyl) sulfonate (CH₂)ₙSO₃H, (alkyl) sulfate (CH₂)ₙOSO₃H, (alkyl) phosphate (CH₂)ₙOP(O)(OH)₂ or (alkyl) phosphonate (CH₂)ₙP(O)(OH)₂, wherein n is an integer ranging from 0 (zero) to 6, or analogs thereof wherein one or more of the CH₂ groups are replaced by CF₂ and/or CD₂;
in particular, R¹-R² (R³-R⁴, R⁵-R⁶) may form a four-, five, six-, or seven-membered cycle (comprising the nitrogen atom) with a hydroxyl group OH, or a phosphorylated hydroxyl group OP(O)(OH)₂ attached to one of the carbon atoms in this cycle, a primary amino group NH₂, secondary amino group NHR^{a} (where R^{a}═C₁-C₆ alkyl of functionally substituted alkyl group), or the combination of such groups;
with the proviso that in all compounds of Formula A at least one, preferably at least 2, 3, or 4, 5 or 6 negatively charged (anionic) groups are present under basic conditions (preferably in aqueous solutions), i.e. 7<pH<14, and these negatively charged groups represent at least partially deprotonated residues of ionizable groups selected from the following set: OH, SH, COOH, a sulfonic acid residue SO₃H, a sulfate residue OSO₃H, an N-cyanosulfonamide residue SO₂NHCN, a phosphate group OP(O)(OH)₂, a phosphonate group P(O)(OH)₂;

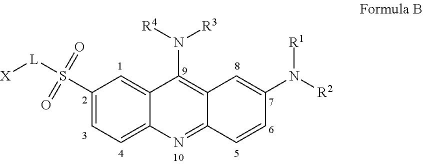

wherein
R¹, R², R³, R⁴ are independent from each other and may represent:
(a) hydrogen;
(b) CH₃, C₂H₅, a straight or branched C₃-C₆, preferably C₃-C₄, alkyl, deuterated alkyl, or perfluoroalkyl group;
(c) straight or branched alkyl group (C₂-C₆) that is unsubstituted, substituted with 1 to 3 halogen atoms (in particular F, Cl, Br), or incorporates 1 to 3 heteroatoms (in particular O, S, N), or optionally substituted with solubilizing groups (OH, NH₂, SH, COOH) and/or ionizable anionic residues, in particular consisting of or including a moiety selected from the group comprising (alkyl) carboxy (CH₂)ₙCO₂H, (alkyl) sulfonate (CH₂)ₙSO₃H, (alkyl) sulfate (CH₂)ₙOSO₃H, (alkyl) phosphate (CH₂)ₙOP(O)(OH)₂ or (alkyl) phosphonate (CH₂)ₙP(O)(OH)₂, wherein n is an integer ranging from 0 (zero) to 6, or analogs thereof wherein one or more of the CH₂ groups are replaced by CF₂ and/or CD₂ residues;
(d) (CH₂)ₙCOOR⁷, where n=1-6, preferably 1-4, and R⁷ may be alkyl, in particular C₁-C₆ alkyl, CH₂CN, CH₂CF₃, benzyl, substituted benzyl, polyhaloalkyl, polyhalophenyl, e.g. tetra- or pentafluorophenyl, pentachlorophenyl, 2- and 4-nitrophenyl, N-succinimidyl, sulfo-N-succinimidyl, 1-benzotriazolyl, 9-aza-1-benzotriazolyl or other groups forming an “active” ester of a carboxylic acid;
(e) a carbonate or carbamate derivative (CH₂)ₘOCOOR⁸ or (CH₂)ₘNHCOOR⁸, where m=1-6 and R⁸=lower alkyl (C₁-C₄), benzyl, 9-fluorenylmethyl, CH₂CN, N-succinimidyl, sulfo-N-succinimidyl, 1-benzotriazolyl, phenyl, substituted phenyl group, e.g., 2- or 4-nitrophenyl, pentachlorophenyl, pentafluorophenyl, 2,3,5,6-tetrafluorophenyl, 2-pyridyl, 4-pyridyl, pyrimid-4-yl;
(f) (CH₂)ₘNR^{a}R, where m=1-6, preferably 2-4, with a straight or branched alkyl chain; R^{a}, R^{b} are independent from each other and represent hydrogen, C₁-C₄ alkyl and/or C₁-C₄ acyl groups, optionally substituted with a hydroxyalkyl group (CH₂)ₙOH, where n=1-6, with a straight or branched alkyl chain, a phosphorylated hydroxyalkyl group (CH₂)ₚOP(O)(OH)₂, where p=1-6, with a straight or branched alkyl chain;
(g) an alkyl azide (CH₂)_{q}N₃, where q=1-8, preferably 2-6, with a straight or branched alkyl chain;
(h) an alkylalkyne (CH₂)ᵣC<triple bond>CR⁹, where r=1-8, with a straight or branched alkyl chain in (CH₂)ᵣ and R⁹=hydrogen, alkyl C₁-C₆ or (functionally) substituted alkyl C₁-C₆;
(i) (CH₂)ₛCONHR¹⁰, with s=1-8, preferably 1-5; R¹⁰═H, C₁-C₆ alkyl, (CH₂)_{q}N₃, (CH₂)ᵣC<triple bond>CH or (CH₂)ₜ—N-maleimido, (CH₂)ᵤ—NH—COCH₂X (X═Br or I), where q, r, t, u indices are independent in the range 1-8, preferably 2-6, and with straight or branched alkyl chains in (CH₂)ₛ, (CH₂)_{q}, (CH₂)ᵣ, (CH₂)ₜ, (CH₂)ᵤ and R¹⁰;
(j) a primary amino group (NH₂) or secondary amino group (NHR¹¹); R¹¹=alkyl C₁-C₆ or (functionally) substituted alkyl C₁-C₆;
R¹, R², R³, R⁴ may contain a terminal alkyloxyamino group (CH₂)ᵥONH₂, where v=1-12, preferably 2-6, with a straight or branched alkyl chain that can include one or multiple (alkyl)oxy (CH₂)ₖO, (alkyl)amino (CH₂)ₗNH or (alkyl)amido (CH₂)_{w}CONH groups in all possible combinations with indices k, l and w=0-12 (independent from each other);
R¹, R², R³, R⁴ may contain a terminal acylhydrazino group (CH₂)ₓCONHNH₂, where x=0-12, preferably 2-6, with a straight or branched alkyl chain that can include one or multiple (alkyl)oxy (CH₂)ₖO, (alkyl)amino (CH₂)ₗNH or (alkyl)amido (CH₂)_{w}CONH groups in all possible combinations with indices k, l and w=0-12 (independent from each other);
one of the residues R¹, R², R³, R⁴ may represent C(O)R¹² or COOR¹³, where R¹² or R¹³ ═C₁-C₄ alkyl, optionally substituted with a hydroxyalkyl group (CH₂)_{y}OH, where y=1-6, with a straight or branched alkyl chain, a phosphorylated hydroxyalkyl group (CH₂)ₘOP(O)(OH)₂, where m=1-6, with a straight or branched alkyl chain;
further, one of the residues R¹, R², R³, R⁴ may represent (CH₂)_{z}—C₆H₄—NH₂, COC₆H₄—NH₂, CONHC₆H₄—NH₂ or CSNHC₆H₄—NH₂ with z=1-4, and C₆H₄ being a 1,2-, 1,3- or 1,4-phenylene, COC₅H₃N—NH₂ or (CH₂)ₛ—C₅H₃N—NH₂, with z=1-4, and C₅H₃N being pyridin-2,4-diyl, pyridin-2,5-diyl, pyridin-2,6-diyl, or pyridin-3,5-diyl;
additionally, R¹-R² (R³-R⁴) may form a four-, five, six-, or seven-membered cycle (comprising the nitrogen atom); which heterocycle may be unsubstituted or substituted with a straight or branched alkyl group (C₂-C₆), which is unsubstituted, substituted with 1 to 3 halogen atoms (in particular F, Cl, Br) or incorporates 1 to 3 heteroatoms (in particular O, S, N), or optionally substituted with solubilizing groups (in particular OH, NH₂, SH, COOH) and/or ionizable anionic residues, in particular consisting of or including a moiety selected from the following groups: (alkyl) carboxy (CH₂)ₙCO₂H, (alkyl) sulfonate (CH₂)ₙSO₃H, (alkyl) sulfate (CH₂)ₙOSO₃H, (alkyl) phosphate (CH₂)ₙOP(O)(OH)₂ or (alkyl) phosphonate (CH₂)ₙP(O)(OH)₂, wherein n is an integer ranging from 0 (zero) to 6, or analogs thereof wherein one or more of the CH₂ groups are replaced by CF₂ and/or CD₂;
in particular, R¹-R² (R³-R⁴) may form a four-, five, six-, or seven-membered cycle (comprising the nitrogen atom) with a hydroxyl group OH, or a phosphorylated hydroxyl group OP(O)(OH)₂ attached to one of the carbon atoms in this cycle, a primary amino group NH₂, secondary amino group NHR^{a} (where R^{a}═C₁-C₆ alkyl of functionally substituted alkyl group), or the combination of such groups;
with the proviso that in all compounds of Formula A at least one, preferably at least 2, 3, or 4-6 negatively charged groups are present under basic conditions (preferably in aqueous solutions), i.e. 7<pH<14, and these negatively charged groups represent at least partially deprotonated residues of ionizable groups selected from the following set: OH, SH, COOH, a sulfonic acid residue SO₃H, a sulfate residue OSO₃H, an N-cyanosulfonamide residue SO₂NHCN, a phosphate group OP(O)(OH)₂, a phosphonate group P(O)(OH)₂;
the linker L (connecting the dye core with the group X) comprises at least one carbon atom and can be represented by alkyl, heteroalkyl (e. g., alkyloxy: CH₂OCH₂, CH₂CH₂O, CH₂CH₂OCH₂), difluoromethyl (CF₂), alkene or alkyne moieties in any combinations, at any occurrence, linear or branched, with the length ranging from C₁ to C₁₂, which allow further connection to a solubilizing and/or ionizable moieties X, or incorporation of heteroatoms (e.g., O, N, S) into such (a) linker(s);
in particular, the linker L may consist of alkanediyl (C₂-C₁₂), heterosubstituted alkanediyl containing heteroatoms (e.g., 0, N and/or S) in any combination, in particular alkyloxyalkyl (CH₂)ₙO(CH₂)ₘ, alkylthiaalkyl (CH₂)ₙS(CH₂)ₘ, or alkylazaalkyl (CH₂)ₙNR(CH₂)ₘ, where n=1-6, m=1-6 (independent from each other) and R═H or (functionally substituted) alkyl (C₁-C₄);
further on, the linker L can be represented by a divalent residue derived from dialkylamine, particularly diethanolamine or N-alkylmonoethanolamine moieties such as —N(CH₃)CH₂CH₂O— and N(CH₂CH₂O—)₂, difluoromethyl (CF₂), alkene or alkyne moieties in any combinations, at any occurrence, linear or branched, with the number of C-atoms ranging from C₁ to C₁₂;
additionally, the linker L may also include a carbonyl (>CO) moiety, the group CF₂CO, one or several CD₂ groups and/or terminal CD₃ groups;
wherein X denotes a solubilizing and/or ionizable anion-providing moiety, in particular consisting of or including a group selected from hydroxyalkyl (CH₂)ₙOH, thioalkyl (CH₂)ₙSH, carboxy alkyl (CH₂)ₙCO₂H, alkyl sulfonate (CH₂)ₙSO₃H, alkyl sulfate (CH₂)ₙOSO₃H, alkyl phosphate (CH₂)ₙOP(O)(OH)₂ or phosphonate (CH₂)ₙP(O)(OH)₂, wherein n is an integer ranging from 0 to 12 or 1 to 12, or an analogon thereof wherein one or more of the CH₂ groups are replaced by CF₂ and/or CD₂;
with the proviso that in all compounds of Formula B at least one, preferably 2 and 3, or 4-6 negatively charged groups are present under basic conditions, i.e. 7<pH<14, and these negatively charged groups represent at least partially deprotonated residues of ionizable groups selected from hydroxyl OH, thiol SH, carboxyl COOH, a sulfonic acid residue SO₃H, a sulfate residue OSO₃H, an N-cyanosulfonyl group SO₂NHCN, a phosphate group OP(O)(OH)₂, a phosphonate group P(O)(OH)₂.

The structures of the novel 1-aminopyrene fluorescent dyes are selected from compounds having either three cyanamidosulfonyl (C), or (3-hydroxyazetidinyl)sulfonamido (D), or (3-hydroxycyclobutyl)sulfonyl (E) groups in positions 3, 6 and 9, or salts (protonated forms) thereof:

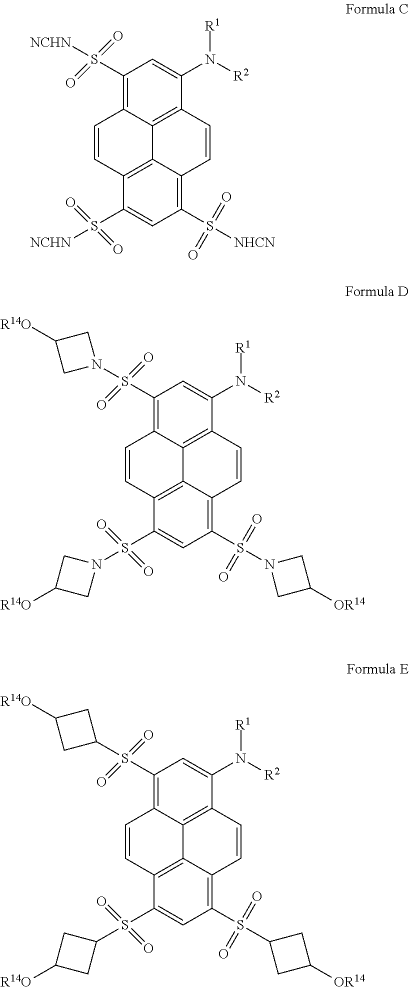

wherein
R¹, R² are independent from each other and selected from the group consisting of
(a) hydrogen;
(b) CH₃, C₂H₅, a straight or branched C₃-C₆, preferably C₃-C₄, alkyl, deuterated alkyl, or perfluoroalkyl group;
(c) straight or branched alkyl group (C₂-C₆) that is unsubstituted, substituted with 1 to 3 halogen atoms (in particular F, Cl, Br), or incorporates 1 to 3 heteroatoms (in particular O, S, N), or optionally substituted with solubilizing groups (in particular OH, NH₂, SH, COOH) and/or ionizable anionic residues, in particular consisting of or including a moiety selected from the group comprising (alkyl) carboxy (CH₂)ₙCO₂H, (alkyl) sulfonate (CH₂)ₙSO₃H, (alkyl) sulfate (CH₂)ₙOSO₃H, (alkyl) phosphate (CH₂)ₙOP(O)(OH)₂ or (alkyl) phosphonate (CH₂)ₙP(O)(OH)₂, wherein n is an integer ranging from 0 (zero) to 6, or analogs thereof wherein one or more of the CH₂ groups are replaced by CF₂ and/or CD₂ residues;
(d) (CH₂)ₙCOOR⁷, where n=1-6, preferably 1-4, and R⁷ may be alkyl, in particular C₁-C₆ alkyl, CH₂CN, CH₂CF₃, benzyl, substituted benzyl, CH₂CF₃, polyhaloalkyl, polyhalophenyl, e.g. tetra- or pentafluorophenyl, pentachlorophenyl, 2- and 4-nitrophenyl, N-succinimidyl, sulfo-N-succinimidyl, 1-benzotriazolyl, 9-aza-1-benzotriazolyl or other groups forming an “active” ester of a carboxylic acid; (e) a carbonate or carbamate derivative (CH₂)ₘOCOOR⁸ or (CH₂)ₘNHCOOR⁸, where m=1-6 and R⁸=lower alkyl (C₁-C₄), benzyl, 9-fluorenylmethyl, CH₂CN, N-succinimidyl, sulfo-N-succinimidyl, 1-benzotriazolyl, phenyl, substituted phenyl group, e.g., 2- or 4-nitrophenyl, pentachlorophenyl, pentafluorophenyl, 2,3,5,6-tetrafluorophenyl, 2-pyridyl, 4-pyridyl, pyrimid-4-yl;
(f) (CH₂)ₘNR^{c}R^{d}, where m=1-6, preferably 2-4, with a straight or branched alkyl chain; R^{c}, R^{d} are independent from each other and represent hydrogen and/or C₁-C₄ alkyl groups, a hydroxyalkyl group (CH₂)ₘOH, where m=2-6, with a straight or branched alkyl chain, a phosphorylated hydroxyalkyl group (CH₂)ₘOP(O)(OH)₂, where m=2-6, with a straight or branched alkyl chain;
(g) an alkyl azide (CH₂)ₘN₃, where m=1-8, preferably 2-6, with a straight or branched alkyl chain;
h) an alkylalkyne (CH₂)ᵣC<triple bond>CR⁹, where r=1-8, with a straight or branched alkyl chain in (CH₂)ᵣ and R⁹=hydrogen, alkyl C₁-C₆ or (functionally) substituted alkyl C₁-C₆.
i) (CH₂)ₛCONHR¹⁰, with s=1-8, preferably 1-5; R¹⁰═H, C₁-C₆ alkyl, (CH₂)_{q}N₃, (CH₂)ᵣC<triple bond>CH or (CH₂)ₜ—N-maleimido, (CH₂)ᵤ—NH—COCH₂X (X═Br or I), where q, r, t, u indices are independent in the range 1-8, preferably 2-6, and with straight or branched alkyl chains in (CH₂)ₛ, (CH₂)_{q}, (CH₂)ᵣ, (CH₂)ₜ, (CH₂)ᵤ and R¹⁰;
j) a primary amino group (NH₂) or secondary amino group (NHR¹¹); R¹¹=alkyl C₁-C₆ or (functionally) substituted alkyl C₁-C₆; R¹, R² may contain a terminal alkyloxyamino group (CH₂)ᵥONH₂, where v=1-12, preferably 2-6, with a straight or branched alkyl chain that can include one or multiple (alkyl)oxy (CH₂)ₖO, (alkyl)amino (CH₂)ₗNH or (alkyl)amido (CH₂)_{w}CONH groups in all possible combinations with indices k, l and w=0-12 (independent from each other); R¹, R² may contain a terminal acylhydrazino group (CH₂)ₓCONHNH₂, where x=1-12, preferably 2-6, with a straight or branched alkyl chain that can include one or multiple (alkyl)oxy (CH₂)ₖO, (alkyl)amino (CH₂)ₗNH or (alkyl)amido (CH₂)_{w}CONH groups in all possible combinations with indices k, l and w=0-12 (independent from each other); one of the residues R¹, R² or both residues R¹ and R² may represent C(O)R¹² or COOR¹³, where R¹² or R¹³═C₁-C₄ alkyl, optionally substituted with a hydroxyalkyl group (CH₂)_{y}OH, where y=1-6, with a straight or branched alkyl chain, a phosphorylated hydroxyalkyl group (CH₂)ₘOP(O)(OH)₂, where m=1-6, with a straight or branched alkyl chain;
further, one of the residues R¹, R² may represent (CH₂)_{z}—C₆H₄—NH₂, COC₆H₄—NH₂, CONHC₆H₄—NH₂ or CSNHC₆H₄—NH₂ with z=1-4, and C₆H₄ being a 1,2-, 1,3- or 1,4-phenylene, COC₅H₃N—NH₂ or (CH₂)ₛ—C₅H₃N—NH₂, with z=1-4, and C₅H₃N being pyridin-2,4-diyl, pyridin-2,5-diyl, pyridin-2,6-diyl, or pyridin-3,5-diyl;
additionally, R¹-R² may form a four-, five, six-, or seven-membered cycle (comprising the nitrogen atom); which (hetero)cycle may be unsubstituted or substituted with a straight or branched alkyl group (C₂-C₆), which is unsubstituted, substituted with 1 to 3 halogen atoms (in particular F, Cl, Br) or incorporates 1 to 3 heteroatoms (in particular O, S, N), or optionally substituted with solubilizing groups (in particular OH, NH₂, SH, COOH) and/or ionizable anionic residues, in particular consisting of or including a moiety selected from the following groups: (alkyl) carboxy (CH₂)ₙCO₂H, (alkyl) sulfonate (CH₂)ₙSO₃H, (alkyl) sulfate (CH₂)ₙOSO₃H, (alkyl) phosphate (CH₂)ₙOP(O)(OH)₂ or (alkyl) phosphonate (CH₂)ₙP(O)(OH)₂, wherein n is an integer ranging from 0 (zero) to 6, or analogs thereof wherein one or more of the CH₂ groups are replaced by CF₂ and/or CD₂;
in particular, R¹-R² may form a four-, five, six-, or seven-membered cycle (comprising the nitrogen atom) decorated with a hydroxyl group OH, or a phosphorylated hydroxyl group OP(O)(OH)₂ attached to one of the carbon atoms in this cycle, a primary amino group NH₂, secondary amino group NHR^{a} (where R^{a}═C₁-C₆ alkyl of functionally substituted alkyl group), or the combination of such groups;
while in a more specific embodiment, in the structures of the novel 1-aminopyrene fluorescent dyes with either three cyanamidosulfonyl (C), or (3-hydroxyazetidinyl)sulfonamido (D), or (3-hydroxycyclobutyl)sulfonyl (E) groups in positions 3, 6 and 9, or salts (protonated forms) thereof
R¹, R² are independent from each other and selected from the group consisting of
a) hydrogen;
b) CH₃, C₂H₅, a straight or branched C₃-C₆ group;
c) aminooxyalkyl groups H₂NO(CH₂)ₙ, wherein n is an integer ranging from 2 to 6, or analogs thereof wherein one or more of the CH₂ groups are replaced by CF₂ and/or CD₂ residues;
d) (CH₂)ₘCOOR⁷, where m=1-6, preferably 1-4, and R⁷ may be H, CH₂CN, CH₂CF₃, CF₃, polyhaloalkyl, polyhalophenyl, e.g. tetra- or pentafluorophenyl, pentachlorophenyl, 2- and 4-nitrophenyl, N-succinimidyl, sulfo-N-succinimidyl, 1-benzotriazolyl, 9-aza-1-benzotriazolyl or other groups forming an “active” ester of a carboxylic acid;
e) a carbonate or carbamate derivative (CH₂)ₘOCOOR⁸ or (CH₂)ₘNHCOOR⁸, where m=1-6 and R⁸═CH₂CN, N-succinimidyl, sulfo-N-succinimidyl, 1-benzotriazolyl, phenyl, substituted phenyl group, e.g., 2- or 4-nitrophenyl, pentachlorophenyl, pentafluorophenyl, 2,3,5,6-tetrafluorophenyl, 2-pyridyl, 4-pyridyl, pyrimid-4-yl; f) an alkyl azide (CH₂)ₘN₃, where m=1-8, preferably 2-6, with a straight or branched alkyl chain;
g) a terminal acylhydrazino group (CH₂)ₓCONHNH₂, where x=1-12, preferably 2-6; with the proviso that in all compounds of Formulae C, D, E at least 3 negatively charged groups are present under basic conditions, i.e. 7<pH<14, and these negatively charged groups represent at least partially deprotonated residues of ionizable groups selected from the following set: OH, SH, COOH, a sulfonic acid residue SO₃H, a sulfate residue OSO₃H, an N-cyanosulfonamide residue SO₂NHCN, a phosphate group OP(O)(OH)₂, a phosphonate group P(O)(OH)₂;
R¹⁴═H or primary phosphate group (P(O)(OH)₂).

The term “substituted” as used herein, generally refers to the presence of one or more substituents, in particular substituents selected from the group comprising straight or branched alkyl, in particular C₁-C₄ alkyl, e.g. methyl, ethyl, propyl, butyl; isoalkyl, e.g. isopropyl, isobutyl (2-methylpropyl); secondary alkyl group, e.g. sec-butyl (but-2-yl); tert-alkyl group, e.g. tert-butyl (2-methylpropyl). Additionally, the term “substituted” may refer here to alkyl groups having at least one deuterium-, fluoro-, chloro- or bromo substituent instead of hydrogen atoms, or methoxy, ethoxy, 2-(alkyloxy)ethyloxy groups (alk-OCH₂CH₂O), and, in a more general case, oligo(ethylenglycol) residues of the art alk(OCH₂CH₂)ₙOCH₂CH₂—, where alk=CH₃, C₂H₅, C₃H₇, C₄H₁₀, and n=1-12.

The term “linker” as used herein, generally refers to a single covalent bond (“zero-linker”) or any divalent residue incorporating 1-20 nonhydrogen atoms selected from the group consisting of C, N, O, S and P that covalently attaches the fluorescent compounds to another entity, such as solubilizing and/or ionizable anion-providing group or a chemically reactive group. Exemplary linkers include any divalent moiety derived from an alkyl, heteroalkyl, in particular, alkyloxy group, and represented, for example, by CH₂OCH₂, CH₂CH₂O, CH₂CH₂OCH₂CH₂. Further, linkers may include any divalent moiety derived from alkylamino or dialkylamino groups; particularly derived from the structures of diethanolamine or N-alkylmonoethanolamine, such as N(CH₃)CH₂CH₂O— and N(CH₂CH₂O—)₂. Linkers may include in their structures difluoromethyl (CF₂), alkene or alkyne moieties in any combinations, at any occurrence, linear or branched, with the length ranging from C₁ to C₁₂.

A “cleavable linker” is a linker comprises one or more “cleavable groups” that may be broken by irradiation with light (a “photocleavable linker”) or in the course of a chemical transformation, including enzymatic reaction. Exemplary enzymatically cleavable groups include natural amino acids or peptide sequences that end with a natural amino acid. Cleavage of a linker breaks at least one chemical bond and releases a (fluorescent) dye or any other “active” part of the initial assembly; e.g., a physiologically active drug, catalyst, inhibitor, acidic or basic component, quencher of the fluorescence signal.

The term “alkyl” refers to any alkyl group selected from the group comprising straight or branched alkyl, more specifically C₁-C₂₀ alkyl, C₁-C₁₂ alkyl, or C₁-C₆ alkyl, e.g. methyl, ethyl, propyl, butyl; isoalkyl, e.g. isopropyl, isobutyl (2-methylpropyl); secondary alkyl group, e.g. sec-butyl (but-2-yl); tert-alkyl group, e.g. tert-butyl (2-methylpropyl) etc.

The terms “aromatic heterocyclic group” or “heteroaromatic group”, as used herein, generally refer to an unsubstituted or substituted cyclic aromatic radical (residue) having from 5 to 10 ring atoms of which at least one ring atom is selected from S, O and N; the radical being joined to the rest of the molecule via any of the ring atoms. Representative, but not limiting examples are furyl, thienyl, pyridinyl, pyrazinyl, pyrimidinyl, pyrrolyl, imidazolyl, thiazolyl, oxazolyl, isooxazolyl, thiadiazolyl, oxadiazolyl, quinolinyl and isoquinolinyl.

The term “groups capable of forming an active ester”, as used herein, generally refers to groups which activate a carboxyl group, making it more reactive with nucleophiles such as, but not limited to, free amino groups of peptides, polyaminoacids, polysaccharides, or analytes under such conditions that no interfering side reactions with other reactive groups of the nucleophile-carrying substance can usefully occur. Examples of such groups that form active esters include N-succinimidyl, sulfo-N-succinimidyl, 1-benzotriazolyl, and the like.

In some specific embodiments of the novel fluorescent dyes according to the present invention, the analyte-reactive group at variable positions R¹, R², R³, R⁴, R⁵, R⁶ in Formula A or the analyte-reactive group at variable positions R¹, R², R³, R⁴ in Formula B may be represented by an aromatic or heterocyclic amine, carboxylic acid, ester of the carboxylic acid (e.g., N-hydroxysuccinimidyl or another amino reactive ester); or represented by alkyl azide (CH₂)ₙN₃, alkyne (e. g., propargyl), strained cyclic alkene, strained cyclic alkyne, amino (NH₂), amino-oxyalkyl (CH₂)ₘONH₂, maleimido (C₄H₃NO₂ with a nucleophile-reactive double bond) or halogeno ketone function (COCH₂X; X═Cl, Br and I), as well as halogeno amide group (NRCOCH₂X, R═H, C₁-C₆-alkyl, X═Cl, Br, I) connected either directly or indirectly via alkandiyl —(CH₂)ₚ—, carbonyl (>CO), amido (—CONR^{a}—), nitrogen (—NR^{b}—), oxygen (—O—) or sulfur-containing linkers (—S—, >SO, >SO₂). R, R^{a}, R^{b} are independent from each other and represent H, lower alkyl group (C₁-C₆), or (functionally substituted) lower alkyl group (C₁-C₆); n, m, p=1-8 (independent from each other).

In some other specific embodiments the aryl amino groups (NR¹R² and/or NR³R⁴) in Formula A or B are connected to an analyte-reactive group via (poly)methylene, poly(ethylene glycol), carbonyl, nitrogen or sulfur-containing linear or branched linkers, particularly (CH₂)ₙCONR (R═H, low (substituted) alkyl), CO(CH₂)ₘNR (R═H, low (substituted) alkyl), CO(CH₂)ₚS(CH₂)ₙ, (CH₂)_{q}S(CH₂)ₙCO, CO(CH₂)ᵣSO₂(CH₂)ₛ, (CH₂)ₜSO₂(CH₂)ᵤCO, their combinations, or linked as a part of nitrogen-containing non-aromatic heterocycles (e.g., piperazines, pipecolines, oxazolines, azetidines); m, n, p, q, r, s, t, u are independent integers ranging from 0 to 12 or 1 to 12; or alternatively the aryl amino groups (NR¹R² and/or NR³R⁴) in Formula A or B are connected to an acyl hydrazine or alkyl hydrazine moiety indirectly, via linkers, thus comprising hydrazides (ZCONHNH₂) or hydrazines (ZNHNH₂), respectively, wherein Z denotes the dye residue of Formula A or B that includes aryl amino groups and linkers;

in particular, R¹ and R³ may be represented by: (CH₂)ₘCONR, CO(CH₂)ₙNR (R═H, low (substituted) alkyl), CO(CH₂)ₚS(CH₂)ₙ, (CH₂)_{q}S(CH₂)ᵣCO, CO(CH₂)ₛSO₂(CH₂)ₜ, (CH₂)ᵤSO₂(CH₂)_{w}CO and their combinations; m, n, p, q, r, s, t, u, w are independent integers ranging from 0 to 12;
linkers may also be represented by non-aromatic O, N and S-containing heterocycles, e. g., piperazines, pipecolines, azetidines.

A further specific embodiment of the invention relates to a fluorescent dye or dye salt according to formulae A or B above, wherein carbohydrate-reactive groups, particularly hydrazine —N(R)NH₂, hydroxylamine —N(R)OH or aminooxy —ONH₂ groups with R being H, lower alkyl (C₁-C₆), heteroalkyl (e.g. CH₃O(CH₂CH₂)ₙO(CH₂CH₂)ₘ; n, m=1-6), alkenyl (e.g., allyl), or (per)fluoroalkyl (C₁-C₆) are connected to the nitrogen site N(R¹)R² in the dye of formulae A or B via all types of linkers (groups R¹, R², R³, R⁴, R⁵ or R⁶ in structures A and B), or linkers L in formula B.

Some further specific and preferred embodiments of the present invention are described below.

In Formula A above, NR¹R² and/or NR³R⁴ typically comprise or represent carbonyl-reactive groups. Preferably, R¹, R², R³ and R⁴ are represented by hydrogen, a primary amino group (NH₂) or secondary amino group (NHR¹⁰); R¹⁰=alkyl C₁-C₆ or (functionally) substituted alkyl (C₁-C₆); linear or branched alkyl, hydroxyalkyl or perfluoroalkyl groups. Substituents R³, R⁴, R⁵ and R⁶ preferably comprise solubilizing and/or anion-providing groups, particularly hydroxyalkyl ((CH₂)ₙOH), thioalkyl ((CH₂)ₙSH), carboxyalkyl ((CH₂)ₙCO₂H), alkyl sulfonate ((CH₂)ₙSO₃H), alkyl sulfate ((CH₂)ₙOSO₃H), alkyl phosphate ((CH₂)ₙOP(O)(OH)₂) or alkyl phosphonate ((CH₂)ₙP(O)(OH)₂), wherein n is an integer ranging from 1 to 12.

Alternatively, substituents R¹, R², R³, R⁴, R⁵ and R⁶ may be represented by carboxylic acid residues (CH₂)ₙCOOH, where n=1-12, and their reactive esters (CH₂)ₙCOOR⁷ as nucleophile-reactive groups. R⁷ can be alkyl (including tert-butyl), benzyl, 9-fluorenylmethyl, polyhalogenoalkyl, CH₂CN, polyhalogenophenyl (e. g., tetra- or pentafluorophenyl, pentachlorophenyl), 2- and 4-nitrophenyl, N-succinimidyl, sulfo-N-succinimidyl, 1-benzotriazolyl or other groups forming an “active” ester of a carboxylic acid. The alkyl chains (or backbones) (CH₂)ₙ may be linear or branched.

Further, the aryl amino groups (NR¹R² and/or NR³R⁴) in Formula A can be connected to an analyte-reactive group via (poly)methylene, polyethylene glycol, carbonyl, nitrogen or sulfur-containing linear or branched linkers, particularly (CH₂)ₘCONR (R═H, low (substituted) alkyl), CO(CH₂)ₘNR (R═H, low (substituted) alkyl), CO(CH₂)ₘS(CH₂)ₙ, (CH₂)ₘS(CH₂)ₙCO, CO(CH₂)ₘSO₂(CH₂)ₙ, (CH₂)ₘSO₂(CH₂)ₙCO, their combinations, or linked as a part of nitrogen-containing non-aromatic heterocycles (e.g., piperazines, pipecolines, oxazolines, azetidines); m and n are integers ranging from 0 to 12 or 1 to 12.

Alternatively, aryl amino groups (NR¹R² and/or NR³R⁴) in Formula A can be connected to an acyl hydrazine or alkyl hydrazine moiety indirectly, via linkers, thus comprising hydrazides (ZCONHNH₂) or hydrazines (ZNHNH₂), respectively. Here Z denotes the dye residue of Formula A that includes aryl amino groups and linkers. In particular, R¹ and R³ may be represented by: (CH₂)ₘCONR, CO(CH₂)ₘNR (R═H, low (substituted) alkyl), CO(CH₂)ₘS(CH₂)ₙ, (CH₂)ₘS(CH₂)ₙCO, CO(CH₂)ₘSO₂(CH₂)ₙ, (CH₂)ₘSO₂(CH₂)ₙCO and their combinations; m and n are integers ranging from 0 to 12. Linkers may also be represented by non-aromatic O, N and S-containing heterocycles (e. g., piperazines, pipecolines, azetidines).

Further, R¹, R², R³, R⁴ may be represented by CH₂—C₆H₄—NH₂, COC₆H₄—NH₂, CONHC₆H₄—NH₂ or CSNHC₆H₄—NH₂ with C₆H₄ being a 1,2-, 1,3- or 1,4-phenylene, COC₅H₃N—NH₂ or CH₂—C₅H₃N—NH₂, with C₅H₃N being pyridine-2,4-diyl, pyridine-2,5-diyl, pyridine-2,6-diyl, pyridine-3,5-diyl.

The analyte-reactive group at variable positions R¹, R², R³, R⁴, R⁵, R⁶ may be represented by an aromatic or heterocyclic amine, carboxylic acid, ester of the carboxylic acid (e.g., N-hydroxysuccinimidyl or another amino reactive ester); or represented by alkyl azide (CH₂)ₙN₃, alkyne (propargyl), amino-oxyalkyl (CH₂)ₙONH₂, maleimido (C₄H₃NO₂ with a nucleophile-reactive double bond) or halogeno ketone function (COCH₂X; X═Cl, Br and I), as well as halogeno amide group (NRCOCH₂X, R═H, C₁-C₆-alkyl, X═Cl, Br, I) connected either directly or indirectly via carbonyl, amido, nitrogen, oxygen or sulfur-containing linkers listed for hydrazine derivatives where n=1-12.

One especially preferred embodiment of the present invention relates to compounds of Formula A above, where the negative charges are provided by several primary phosphate groups, in particular, triple O-phosphorylated 7,9-diaminoacridine-2-sulfonamide (compound 13).

Further specific and preferred embodiments of the present invention are described by Formula B, where:

L is a divalent linker (CH₂)ₘ (m=2-6) connecting the dye core with one or more solubilizing and/or ionizable moieties X, and the moieties L and X are combined according to the formulae (CH₂)ₘOP(O)(OH)₂ and (CH₂)ₘOH;

NR¹R² and/or NR³R⁴ comprise carbonyl-reactive groups. Preferably, R¹, R², R³ and R⁴ are represented by hydrogen, linear or branched alkyl, hydroxyalkyl or perfluoroalkyl groups. Substituents R³, R⁴ preferably comprise solubilizing and/or anion-providing groups, particularly hydroxyalkyl ((CH₂)ₙOH), thioalkyl ((CH₂)ₙSH), carboxyalkyl ((CH₂)ₙ CO₂H), alkyl sulfonate ((CH₂)ₙSO₃H), alkyl sulfate ((CH₂)ₙOSO₃H), alkyl phosphate ((CH₂)ₙOP(O)(OH)₂) or alkyl phosphonate ((CH₂)ₙP(O)(OH)₂), wherein n is an integer ranging from 1 to 12.

Alternatively, substituents R¹, R², R³, R⁴ may be represented by carboxylic acid residues (CH₂)ₙCOOH, where n=1-12, and their reactive esters (CH₂)ₙCOOR⁷ as nucleophile-reactive groups. R⁷ can be alkyl (including tert-butyl), benzyl, 9-fluorenylmethyl, polyhalogenoalkyl, CH₂CN, polyhalogenophenyl (e. g., tetra- or pentafluorophenyl, pentachlorophenyl), 2- and 4-nitrophenyl, N-succinimidyl, sulfo-N-succinimidyl, 1-benzotriazolyl or other groups forming an “active” ester of a carboxylic acid. The alkyl chains (CH₂)ₙ may be linear or branched.

Further, the aryl amino groups (NR¹R² and/or NR³R⁴) in Formula B can be connected to an analyte-reactive group via (poly)methylene, polyethylene glycol, carbonyl, nitrogen or sulfur-containing linear or branched linkers, particularly (CH₂)ₙCONR, (CH₂)ₘCONR (R═H, low (substituted) alkyl), CO(CH₂)ₘS(CH₂)ₙ, (CH₂)ₘS(CH₂)ₙCO, CO(CH₂)ₘSO₂(CH₂)ₙ, (CH₂)ₘSO₂(CH₂)ₙCO, their combinations, or linked as a part of nitrogen-containing non-aromatic heterocycles (e.g., azetidines, piperazines, pipecolines, oxazolines); m and n are integers ranging from 0 to 12 or 1 to 12.

Substituents R¹, R², R³, R⁴ in Formula B may be also represented by a primary amino group, thus comprising carbonyl-reactive aryl hydrazines, unsubstituted or substituted with solubilizing and/or anion-providing moieties, particularly: hydroxyalkyl (CH₂)ₙOH, thioalkyl ((CH₂)ₙSH), carboxyalkyl ((CH₂)ₙCO₂H), alkyl sulfonate ((CH₂)ₙSO₃H), alkyl sulfate ((CH₂)ₙOSO₃H), alkyl phosphate ((CH₂)ₙOP(O)(OH)₂) or phosphonate ((CH₂)ₙP(O)(OH)₂), wherein n is an integer ranging from 0 to 12 or 1 to 12.

Alternatively, aryl amino groups (NR¹R² and/or NR³R⁴) in Formula B can be connected to an acyl hydrazine or alkyl hydrazine moiety indirectly, via linkers, thus comprising hydrazides (ZCONHNH₂) or hydrazines (ZNHNH₂), respectively. Here Z denotes the dye residue of Formula B that includes aryl amino groups and linkers. In particular, R¹ and R³ may be represented by: (CH₂)ₘCONR, CO(CH₂)ₘNR (R═H, low (substituted) alkyl), CO(CH₂)ₘS(CH₂)ₙ, (CH₂)ₘS(CH₂)ₙCO, CO(CH₂)ₘSO₂(CH₂)ₙ, (CH₂)ₘSO₂(CH₂)ₙCO and their combinations; m and n are integers ranging from 0 to 12. Linkers may also be represented by non-aromatic O, N and S-containing heterocycles (e. g., piperazines, pipecolines, azetidines).

Further, R¹, R², R³, R⁴ may be represented by CH₂—C₆H₄—NH₂, COC₆H₄—NH₂, CONHC₆H₄—NH₂ or CSNHC₆H₄—NH₂ with C₆H₄ being a 1,2-, 1,3- or 1,4-phenylene, COC₅H₃N—NH₂ or CH₂—C₅H₃N—NH₂, with C₅H₃N being pyridine-2,4-diyl, pyridine-2,5-diyl, pyridine-2,6-diyl, pyridine-3,5-diyl.

The analyte-reactive group at variable positions R¹, R², R³, R⁴ may be represented by an aromatic or heterocyclic amine, carboxylic acid, ester of the carboxylic acid (e.g., N-hydroxysuccinimidyl or another amino reactive ester); or represented by alkyl azide (CH₂)ₙN₃, alkyne (propargyl), amino-oxyalkyl (CH₂)ₙONH₂, maleimido (C₄H₃NO₂ with a nucleophile-reactive double bond) or halogeno ketone function (COCH₂X; X═Cl, Br and I), as well as halogeno amide group (NRCOCH₂X, R═H, C₁-C₆-alkyl, X═Cl, Br, I) connected either directly or indirectly via carbonyl, amido, nitrogen, oxygen or sulfur-containing linkers listed for hydrazine derivatives where n=1-12.

In preferred embodiments, the fluorescent dye according to claim 1 of Formulae A or B above has a negative net charge q of −1; preferably −3, −5, or higher; the fluorescent dye of Formula C has a net charge z of −3, the fluorescent dye of Formulae D or E above has a net charge z of 0, −3, or −6, preferably −6; in particular in an aqueous medium at pH ranging from 7 to 13. Compounds of Formulae A-E can exist and be applied in the form of salts that involve all possible types of cations, preferably Na⁺, K⁺, Li⁺, NH₄⁺, organic ammonium (e.g. tetraalkylammonium) or organic phosphonium cations.

In more specific embodiments, a fluorescent dye salt according to the present invention may comprise negatively charged N-cyanamidosulfonate and/or phosphate groups and counterions selected from inorganic or organic cations, preferably alkaline metal cations, preferably Na⁺, K⁺, Li⁺, ammonium cations or cations of organic ammonium and phosphonium compounds cations (such as tetraalkylammonium cations), and/or comprising a positively charged group or a charge-transfer complex formed at the nitrogen site N(R¹)R² (with R¹ and R² as defined for formulae A-E above) in the dye of formulae A-E with a counterion selected from anions of a strong mineral, organic or a Lewis acid (such as BF₃).

In an especially preferred embodiment, the fluorescent dye of the general Formula A as defined above has one of the following formulae, including salts or protonated forms thereof:

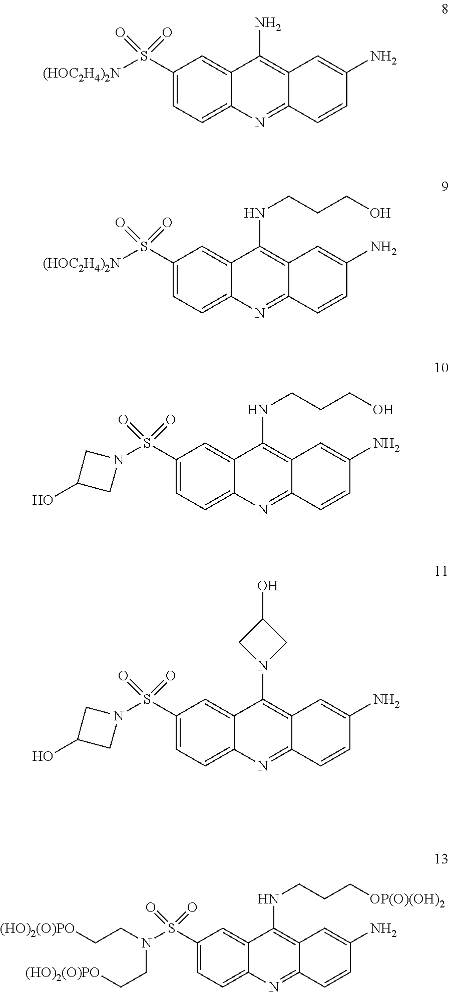

In another especially preferred embodiment, the fluorescent dye of the general Formula B as defined above has one of the following formulae, including salts or protonated forms thereof:

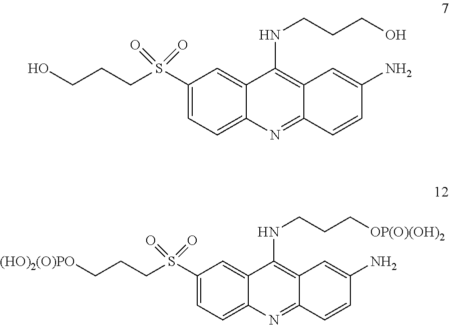

In another especially preferred embodiment, the fluorescent dye of the general Formulae C-E as defined above has one of the following formulae, including salts or protonated forms thereof:

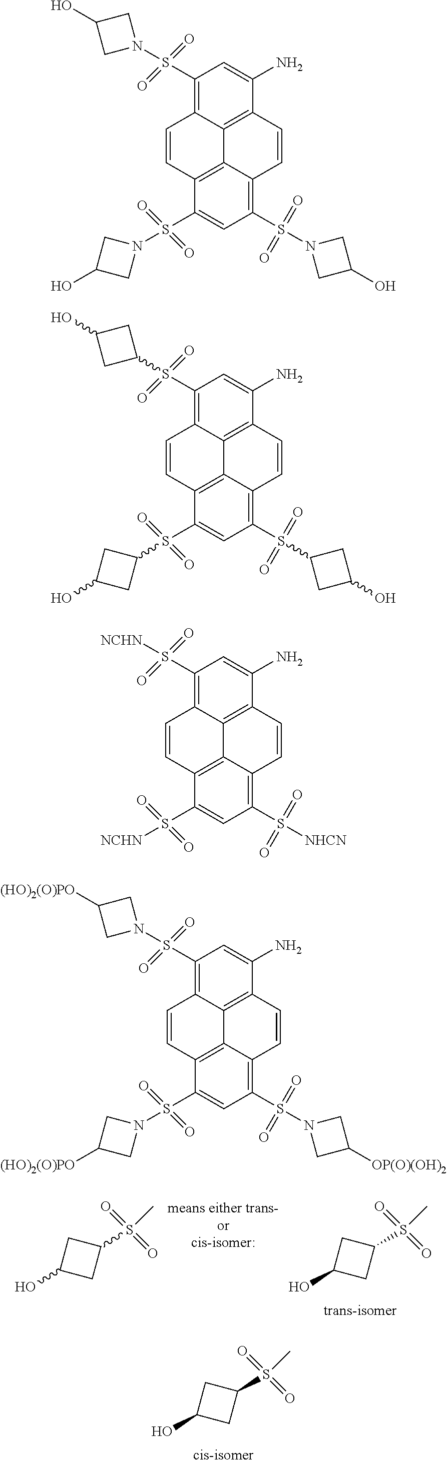

The novel fluorescent dyes of the present invention exhibit favorable properties justifying their use as fluorescent tags (derivatization reagents) facilitating separation of the analytes and their sensitive detection by fluorescence:

they possess either aromatic amino group (ArNH₂), or hydrazine group (NRNH₂, R═H, (substituted) alkyl), hydrazide (CONRNH₂, R═H, (substituted) alkyl), alkoxyamino group (R′ONH₂, R′=substituted alkyl, or L-linker as defined below) for efficient and “clean” (with minimal side products) reductive amination at pH˜2-5 or direct condensation with aldehydes (carbohydrates);
preferably, the aromatic amino group is primary (ArNH₂), but it can also be a secondary one (ArNHR; R=(substituted) alkyl C₂-C₆);
multiple net electrical charges in conjugates (derivatives obtained from biomolecules and reactive dyes)—in the range of −3 to −6 at pH=7-10;
high solubility in aqueous (buffered) solutions at pH 3-10;
high molar absorptivity (extinction coefficient) providing efficient excitation with an argon ion laser (emitting at 488 and 514 nm), 488 nm laser, or 505 nm solid state laser; high brightness (the product of the fluorescence quantum yield and molar absorptivity at the excitation wavelength; e.g., 488 nm or 505 nm);
chemical stability of the dye core against reduction with reducing agents, in particular, borane-based reagents (e.g., sodium cyanoborohydride, 2-picoline borane) at pH=2-6; reactivity towards aldehydes, in particular, reducing carbohydrates and their mixtures (i.e. dextran ladder) under reductive amination conditions;
low emission at ca. 520 nm of their conjugates with aldehydes (carbohydrates) enabling to avoid spectral cross-talk, i.e. simultaneous detection, with APTS-labeled glycans;
high electrophoretic mobility of their conjugates with aldehydes (carbohydrates), outperforming corresponding APTS-labeled glycans;
easy purification resulting in high purity (>99%), as controlled by HPLC and/or electrophoresis.

Summarizing, the present invention provides new 9-aminoacridine dyes with an additional amino group (at C-2), multiple negative charges and red emission. The acridine dyes feature (functionally substituted) sulfonamide or alkylsulfone residues, as strong electron-acceptor groups. The present invention further provides yellow-emitting 1-aminopyrene dyes with six negative charges, as well as 1-aminopyrene dyes with three negatively charged N-cyanosulfonamide residues, which also have sulfonamide or alkylsulfone residues, as electron-acceptor groups providing bathochromic and bathofluoric shifts.

The usefulness of all new dyes for reductive amination and fluorescence detection of glycans is demonstrated by preparation of conjugates with carbohydrates, electrophoresis and fluorescence detection. The spectral properties of dyes and their conjugates (absorption and emission spectra, brightness, stability, reductive amination capacities, electrophoretic mobilities) have been exemplified as shown below.

The novel compounds of the present invention have small molecular size and, in preferred embodiments, a low (negative) net charge (z) at pH>7: such as z=−3 for phosphorylated 7,9-diaminoacridine-2-sulfones (Formula B), z=−3 for pyrene dyes of Formula C, z=−5 for phosphorylated 7,9-diaminoacridine-2-sulfonamides (Formula A), as well as −6 for phosphorylated pyrene dyes of the Formulae D, E. These properties (small molecular size and high negative charge) are equivalent to low mass to charge ratios (m/z), typically in the range from 130 to 199, and a small hydrodynamic radius. As a result, high electrophoretic mobilities and fast separation times can be achieved for these compounds and their conjugates with carbohydrates.

The negative charges are provided by acidic groups which can be deprotonated in basic or even neutral media. Phosphate groups are preferred for this purpose, because primary alkyl phosphates (R—OPO₃H₂) have pKₐ values for the first and the second acidic protons in the range of 1-2 and 6-7, respectively. As a consequence, one single phosphate group can introduce two negative charges in buffer solutions under basic conditions (e.g., at pH 8 and above, when one group R—OPO₃₂— is present), provided that no other basic (proton-acceptor) group is present in the structure. To achieve the negative charge of −4, the attachment of two phosphate groups is necessary, etc. Other acidic groups, for example, N-cyanosulfonamide residues SO₂NHCN are suitable for providing one negative charge.

In a broader scope, compounds of Formulae A-E decorated with additional reactive groups (see text below) are suitable for the use as fluorescent labels for natural products: amino acids, peptides, proteins, including primary and secondary antibodies, single-domain antibodies, taxanes (docetaxel, cabazitaxel, larotaxel), avidin, streptavidin and their modifications, aptamers, nucleotides, nucleic acids, toxins, lipids, carbohydrates, including 2-deoxy-2-aminoglucose and other 2-deoxy-2-aminoaminopyranosides, glycans, biotin, and other so-called “small molecules”, i.e. chemical entities having well-defined known structures with molecular masses of less than 1500 Da, e.g., jasplakinolide and its modifications.

A further aspect of the present invention relates to compounds of formulae A-E and salts thereof for use as a fluorescent label for natural products; e.g., amino acids, peptides, proteins, including primary and secondary antibodies, single-domain antibodies, avidin, streptavidin and their modifications, aptamers, nucleotides, nucleic acids, toxins, lipids, carbohydrates, including 2-deoxy-2-amino glucose and other 2-deoxy-2-aminoaminopyranosides, glycans, biotin, and other small molecules, i.e. having molecular masses of less than 1500 Da, e.g., docetaxel, cabazitaxel, larotaxel, aminophalloidin, jasplakinolide and their modifications.

The claimed compounds are suitable for and may be used as fluorescent reagents for conjugation to analytes, wherein the conjugation comprises formation of at least one covalent chemical bond or at least one molecular complex with a chemical entity or substance, such as amine, carboxylic acid, aldehyde, alcohol, aromatic compound, heterocycle, dye, amino acid, peptide, protein, carbohydrate, nucleic acid, toxin and lipid, followed by fluorescence detection, which can be used alone or combined with any other detection method (e.g., mass-spectrometric detection).

The claimed compounds are in particular suitable for use in the reductive amination and in the conjugation with reducing sugars, i.e. monomeric, oligomeric or polymeric carbohydrates possessing an aldehyde group in a free form or as hemiacetal, including glycans.

Consequently, closely related aspects of the present invention relate to this use and methods for the reductive amination of and/or conjugation with reducing sugars.

In one embodiment, a method for obtaining of dyes' conjugates with reducing sugars, in particular with a mixture of reducing mono-, di- and oligosaccharides with incremental addition of monomeric units and stepwise increasing molecular masses, (so called fluorescent “sugar ladders”) is based on a two-step procedure: 1) formation (and optionally isolation) of the (protonated) imine intermediate (Schiff's base) prepared from the fluorescent dye (8-aminopyrene-1,3,6-trisulfonic acid (APTS), the salt thereof, or any dye according to Formulae A-E and an unlabeled mixture of reducing mono-, di- and oligosaccharides with incremental addition of monomeric units and stepwise increasing molecular masses (unlabeled “sugar ladder”), 2) reduction of the said intermediate.

More specifically, said method for obtaining of dyes' conjugates with reducing sugars comprises a step 1), in which the formation of the (protonated) imine intermediate (Schiff's base) is facilitated by removal of water and high-boiling solvent from the reaction solution containing: a) APTS, the salt thereof, or any dye according to Formulae A-E, b) a reducing sugar of a mixture of reducing sugars; c) acid with pKa=2-6.

In another specific embodiment of the claimed method for obtaining of dyes' conjugates with reducing sugars a dye (APTS, the salt thereof, or any dye according to Formulae A-E), an individual reducing sugar (e.g. glucose or its oligomers) or a “sugar ladder” as defined above are dissolved in water, combined with an organic acid (e.g., 5-20 equiv. of citric, malonic, or malic acid) dissolved in DMSO, incubated at elevated temperature (25-70° C. for 0.5-2 h), followed by removal of solvents under reduced pressure (p<0.2 mbar).

In a still more specific embodiment of the above method, a dye (10 μL of 0.1 M solution in water) is mixed with a dextran ladder (1.0 mg, maltodextrin oligosaccharides—DP2 to DP15, Carbosynth) and malonic acid (10 equiv., 10 μL of 1 M solution in DMSO) followed by incubation at 40° C. for 1 h, removal of the solvents under reduced pressure (p<0.2 mbar), addition of a solution of 2-picoline-borane complex (10 equiv., 10 μL of 1 M solution in DMSO), incubation at 40° C. for 16 h and, finally, isolation of the products.

A further important aspect of the present invention relates to carbohydrate-dye conjugates comprising fluorescent dyes according to Formulae A-E, for example obtainable by the methods described above.

Typically, in said carbohydrate-dye conjugates the carbohydrate moiety is selected from the group comprising or consisting of reducing glycans, such as mannose, N-acetylglucosamine and N-acetylgalactosamine residues, galactose, fucose, glucose, maltose and its oligomers (e.g. maltotriose, maltotetraose, maltopentaose, maltohexaose, maltoheptaose, higher maltose oligomers), as well as sialic acids in various possible combinations.

In a more specific embodiment, the carbohydrate-dye conjugate is selected from the following structures:

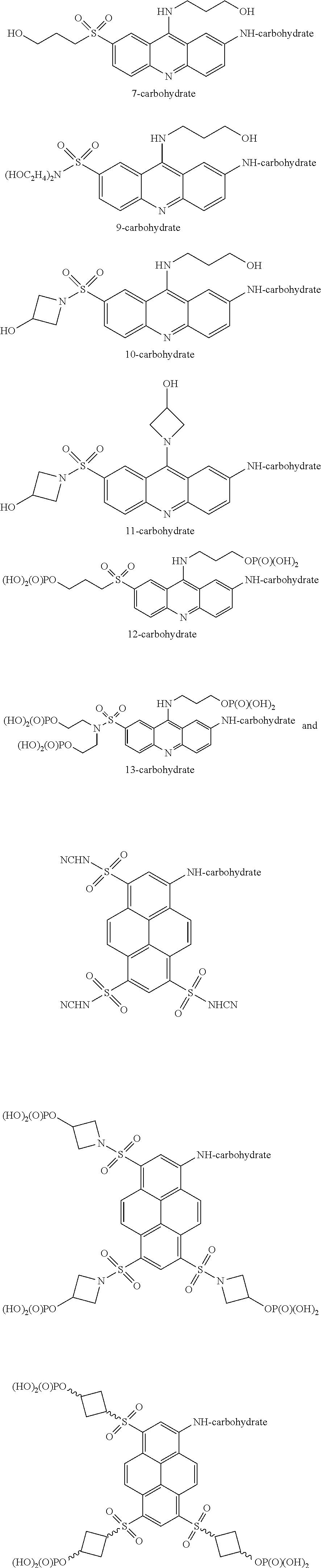

wherein the carbohydrate moiety is selected from the group comprising or consisting of reducing glycans, such as mannose, N-acetylglucosamine and N-acetylgalactosamine residues, galactose, fucose, glucose, maltose and their oligomers (e.g. maltotriose, maltotetraose, maltopentaose, maltohexaose, maltoheptaose, higher maltose oligomers), as well as sialic acids in various possible combinations.

One preferred embodiment of this aspect of the present invention relates to compounds of Formula A above, where the negative charges are provided by several primary phosphate groups, in particular, triple O-phosphorylated 7,9-diaminoacridine-2-sulfonamide. i.e. compound 13. Their glycoconjugates emit red light and have high electrophoretic mobilities. This preferred embodiment of the present invention is in particular represented by the compounds below, where “carbohydrate” denotes reducing glycans (e.g., containing mannose, N-acetylglucosamine and N-acetylgalactosamine residues, galactose, fucose, glucose, maltose and its oligomers (e.g. maltotriose, maltotetraose, maltopentaose, maltohexaose, maltoheptaose, higher maltose oligomers), as well as sialic acids in various possible combinations).

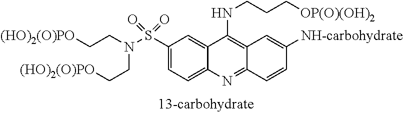

A still further aspect of the present invention relates to a kit or composition comprising one or more of the dyes of Formulae A-E and/or one or more carbohydrate conjugates as disclosed above.

According to the state of the art, glycans may be identified by comparison of their migration times (on the electrophoretogram) against a dextran ladder standard (i.e., fluorescently labeled dextran carbohydrate oligomers differing by one glucose molecule). The dextran ladder may be run in parallel with the glycan samples, and specific glycans may then be identified by locating the time point at which they elute relative to the dextran ladder. Known retention times for specific glycan structures and molecular weights may previously be recorded in an empirically-derived database, which may then be searched. An analysis software may then compare retention times (relative to the dextran ladder) of peaks from an electrophoretogram of unknown glycans with the retention time database to identify the glycans.

Thus, a further aspect of the invention relates to a method of characterizing a sample containing one or more glycans, the method comprising:

a) providing a sample containing one or more glycans (conjugated with a fluorescent dye “A”) as analytes of unknown structure and quantity (concentration) and a known quantity of a fluorescent reference glycan standard, wherein the reference glycan standard is labeled with a dye (“B”) according to Formulae A-E above;
b) contacting (e.g., mixing, co-injecting, etc.) the sample with a dye “B” according to Formulae A-E above conjugated with known (reference) glycans that is different from the dye “A” (fluorescent dye conjugated with one or more unknown glycans) used in step (a);
c) separating the sample by electrophoresis or HPLC;
d) selective (separate) detection of the analytes and reference glycans due to different emission signatures of dyes “A” and “B” and
e) quantifying at least one glycan in the sample relative to the reference glycan standard.

The fluorescent dye “A” may be any dye suitable for conjugation with glycans, in particular any fluorescent dye commonly used for this purpose including ATPS, and may also include a compound of formulae A-E above, provided it is not identical with the compound used as dye “B” of the reference standard used in this method.

A still further aspect of the present invention relates to the compounds according to Formulae A-E or the carbohydrate-dye conjugates comprising the same for the use for spectral calibration of a fluorescence detector, in particular a detector for detection of laser induced fluorescence (LIF) or capillary gel electrophoresis with detection by laser induced fluorescence (CGE-LIF).

The present invention further relates to a method for spectral calibration of a fluorescence detector which comprises injecting a solution containing 2 or more fluorescent dyes according to Formulae A-E above or carbohydrate-dye conjugates comprising the same into an electrophoresis device followed by separation and detection, in particular multichannel detection, of light emitted by each component of the dye mixture by a CCD camera, diodes array detector or similar device providing the spectral resolution and separate detection of the emitted light.

General synthesis and characterization of the novel 7,9-aminoacridine dyes of the invention and their precursors

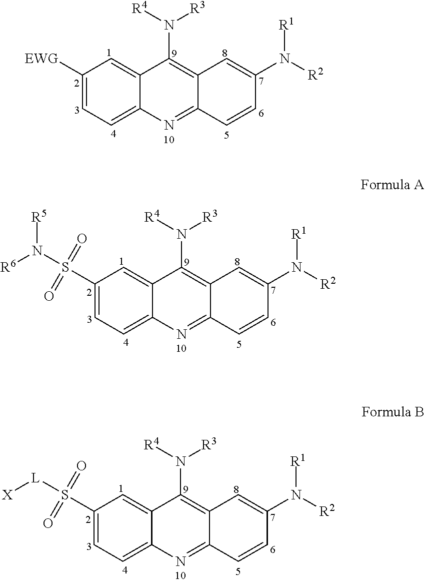

The generic structure of 7,9-diaminoacridines with electron-withdrawing group (EWG) in 2-position exemplified by sulfonamides (Formula A) and sulfones (Formula B) are shown above. Model 7,9-diaminoacridines (6-11) with a strong electron withdrawing group (EWG) in 2-position and ω-hydroxyalkyl substituents were prepared and applied in reductive amination of glucose (FIGS. 9 and 11-18). This allowed to prove the concept; first of all, the reactivity of the primary amino group and spectral properties of the products. The presence of the two electron-donating amino groups and two electron acceptor groups (acridine nitrogen in 10-position and another acceptor in 2-position) leads to dyes with “push-pull” π-systems exhibiting red-shifted absorption and emission bands. Indeed, when the new glycoconjugates (6-7-Glc, 9-11-Glc) were prepared, they were found to absorb and emit light at about 470 nm and 610-630 nm, respectively (in aqueous buffer, see Table 3 for the details). The spectral properties of the dyes (6-11) are given in Table 2.

After establishing the applicability of 2,9-diaminoacridines for labeling and fluorescence detection of reducing sugars, we decorated compounds 7 and 9 with two and three primary phosphate groups, respectively (see structures 12 and 13 in FIGS. 10 and 18). The conjugates with glucose, maltotriose and maltoheptaose were obtained via reductive amination (FIG. 19). The excitation with 488 nm light (and even 505 nm light) is possible (as illustrated by FIG. 1, the absorption bands are broad). The Stokes shifts turned to be large (ca. 150 nm), and the red emission enabled detection by fluorescence without cross-talk with emission provided by APTS conjugates (see below). The spectral properties of the dyes (12, 13) and their glycoconjugates are given in Table 4. The conjugates of compounds 12 and 13 have high electrophoretic mobilities. In particular, conjugates of compound 13 move faster than the corresponding APTS and VBDP (reference acridone dye) derivatives (FIG. 2, FIG. 3, Table 5).

The key features required for the superior fluorescent tags applicable in the reductive amination and C(G)E-LIF of glycans can be summarized as follows: an amino group with relatively low basicity (e.g., aromatic, RONH₂) providing efficient reductive amination at pH˜ 3; the net charge in the range of −3 . . . -6 at pH˜8 (pH of the buffer solution in CE) enabling high electrophoretic mobility; solubility in aqueous buffers and stability against reduction with boranes or borohydrides within a wide range of pH (3-8); efficient “green” excitation (high absorption at 488 nm, ε₄₈₈, or 505 nm, ε₅₀₅); brightness (product of the fluorescence quantum yield and ε₄₈₈ or ε₅₀₅); minimal cross-talk with the “APTS channel” in the detector (low emission of conjugates at 520 nm). These properties follow from the conditions of the reductive amination (FIG. 6), separation and detection of the fluorescent glycan derivatives using the standard DNA sequencing equipment.

Previously, yellow-orange emitting 7-aminoacridone-2-sulfonamides for fluorescence detection of glycans were reported ((PCT/EP2019/051396; e.g.VBDP in FIG. 10). In said application, a red shift of ca. 60 nm was reported in comparison to unsubstituted 2-aminoacridone (AMAC). These electron deficient AMAC derivatives participate in reductive amination reactions with saccharides, have high net charge (z=−4), which is provided by two primary phosphate groups (R—OPO₃H₂), and low m/z ratio (˜140). The first and second pKa values of a primary phosphate group are in the range of 1.5-1.9 and 6.3-6.8, respectively. In the electrophoresis buffer solution (pH˜8), one primary phosphate group introduces two negative charges.

The principles of molecular design outlined above are based on the combination of at least one electron-donor residue (e.g., an amino group) and one electron-acceptor group (e.g., sulfonamide) attached to the acridone system and interacting with each other (and the acridone scaffold) in a “push-pull” fashion. These principles can be applied to 9-aminoacridines to obtain dyes with red shifts in absorption and emission bands. Skripkina et al. reported that absorption bands of 9-aminoacridine-2-sulfonamides with electron-donating methoxy group in 7-position are red-shifted in comparison to the corresponding acridone (V. T. Skripkina, N. N. Dykhanov, V. P. Maksimets, L. D. Shcherbak, Chem. Heterocycl. Compd. 1971, 7, 107-109). A. Szymanska et al. compared 9-aminoacridine and acridone chromophores and reported the emission of acridines to be red-shifted (A. Szymanska, W. Wiczk, L. Lankiewicz, Chem. Heterocycl. Compd. 2000, 36, 801-808). P. Yang et al. described a chromophore based on 9-aminoacridine which showed longer absorption and fluorescence wavelengths than the corresponding acridone (P. Yang, Q. Yang, X. Qian, L. Tong, X. Li, J. Photochem. Photobiol. B, Biol. 2006, 84, 221-226).

The amino group at C-9 in acridine can participate in reductive amination (G. Gellerman, V. Gaisin, T. Brider, Tetrahedron Lett. 2010, 51, 836-839), though it is less reactive than the amino group attached to C-7. In order to exclude any competition between two amino groups (at C-7 and C-9), we decided to shield the amino group in 9-position by alkylation (see E. W. Baxter, A. B. Reitz, in Organic Reactions, 2002, pp. 1-125, doi:10.1002/0471264180.or059.01). The monoalkylation is preferred, since 9-(monoalkylamino)acridines were reported to be more hydrolytically stable than tertiary amines with a 9-(dialkylamino) group (J. R. Goodell, B. Svensson, D. M. Ferguson, J. Chem. Inf Model. 2006, 46, 876-883). The spectral properties of an acridine system and the reactivity of an amino group at C-7 are sensitive to the nature of an acceptor group in 2-position. The electron-acceptor properties of the substituents can be assessed and compared on the basis of their σ-constants in the Hammett equation (Table 1). The present inventors incorporated the strong electron-acceptor groups—cyano, sulfone and sulfonamide—into 7,9-diaminoacridine scaffold. The sulfone and sulfonamide groups are preferred, as they allow to introduce a hydroxyl-substituted side chain, which can be easily phosphorylated. As it was already mentioned above, first acridines 6-11 without phosphate groups (see FIG. 9) were prepared. This approach allowed to study the spectral properties (the required red-emission was achieved), and then carry out the reductive amination successfully.

TABLE 1
Hammett constants for donor and acceptor groups
in meta (σₘ) and para (σₚ) positions.
The positive values correspond to electron-acceptors; negative values—to
electron-donor groups. For hydrogen, σₘ = σₘ = 0 (C. Hansch,
A. Leo, R. W. Taft, Chem. Rev. 1991, 91, 165-195).

| Name | Substituent | σₘ | σₚ |
|---|---|---|---|
| Amino | —NH₂ | −0.16 | −0.66 |
| Methylamino | —NHMe | −0.21 | −0.70 |
| Dimethylamino | —NMe₂ | −0.16 | −0.83 |
| Methylthio | —SMe | 0.15 | 0.00 |
| Methyl sulfonyl | —SO₂Me | 0.60 | 0.72 |
| Methylsulfoxide | —SOMe | 0.52 | 0.49 |
| Sulfonamide | —SO₂NH₂ | 0.53 | 0.60 |
| Nitrile | —CN | 0.56 | 0.66 |

Synthesis of Model Compounds

The universal acridine precursor 18 was prepared in four steps in a good overall yield starting from 5-nitro-N-phenylanthranilic acid 14 (FIG. 11).

2-Amino-7-cyano-9-(6-hydroxyhexyl)aminoacridine (6) with a cyano group at 7-position was synthesized in three steps to evaluate the suitability of 2-aminoacridines with a strong acceptor at C-7 for fluorescent labelling of reducing saccharides (FIG. 12). Model compound 6 was stable as a TFA salt, but was found to be unstable under neutral and basic conditions (formation of insoluble black precipitate was observed). Therefore, in order to evaluate photophysical properties of dye 6, we prepared its conjugate with glucose 6-Glc (FIG. 16). It turned out to be stable under neutral and basic conditions and has an absorption maximum at 471 nm, when dissolved in aqueous buffer (pH 8). Upon excitation, solutions of 6-Glc emit orange light with the maximum at 611 nm. The attractive spectral properties of the model conjugate 6-Glc motivated us to prepare further derivatives (7-11) with various acceptors at C-7, substituted amino group at C-9 and a free amino group at C-2.

Acridine 7 with an electron-withdrawing sulfone substituent at C-7 was prepared in four steps (FIG. 13). Sulfone 7 has two primary hydroxyl groups available for phosphorylation. In contrast to nitrile 6, sulfone 7 was stable in neutral and basic solutions, which allowed to characterize its photophysical properties (Table 2).

Next, the synthesis of acridine-7-sulfonamides 8 and 9 bearing a primary and a secondary amino group in 9-position, respectively, was explored (FIG. 14). Both compounds 8 and 9 are stable under neutral and basic conditions.

The inventors used tertiary sulfonamides as acceptor groups. Primary and secondary sulfonamides were avoided, because the acidity of SO₂NH₂ and SO₂NHR groups in acridines might be high enough, which would lead to partial deprotonation at pH˜ 8, and this could broaden the peaks (bands) in electrophoresis. In an attempt to prepare 3-hyrdoxyazetidine derivative 31 (FIG. 15) in a similar way as described above for the synthesis of 30, the inventors observed transamination which gave compound 32 substituted in 9-position with the amino group of 3-hydroxyazetidine (FIG. 15). The reduction of the nitro group in compounds 31 or 32 afforded amines 10 or 11, respectively. Both dyes 10 and 11 are stable in neutral and basic aqueous solutions.

### Spectroscopic Properties of 2,9-Diaminoacridines

Compounds 7-11 have a free amine group intended for the reductive amination of glycans (see next section). Before performing this reaction, the inventors measured the absorption and fluorescent spectra of the free dyes in aqueous buffer (25 mM HEPES, pH 8) and methanol. The aqueous buffer (pH 8) is highly relevant for the analysis of glycan conjugates using gel electrophoresis. The emission was observed from all dyes 7-11, and the spectral properties are given in Table 2. The absorption spectra of compounds 7-11 in aqueous buffer feature a broad band centered at λ=455-460 nm. When excited in this low-energy absorption band, an intense fluorescence centered at λ=603-626 nm was observed with quantum yields of 2-5%. Dyes 7-11 have particularly large Stokes shifts (156-166 nm) in aqueous buffer, and large Stokes shifts are associated with low emission efficiencies. Changing the solvent to methanol resulted in smaller Stokes shifts of 111-120 nm and larger quantum yields of 17-25%. Comparing sulfonamides (9, 10) with the structurally related alkyl sulfone 7, no significant difference in the position of absorption and emission bands was observed. The bathochromic shift was observed in absorption and fluorescence spectra, when the degree of alkylation of the amino group at C-9 increased. Compared to APTS (emission maximum at 503 nm in aqueous solutions), a remarkable red-shift of 100-113 nm was achieved. The solubility in aqueous media, orange-red emission, and a free amine group on their core structure are important features enabling the use of compounds 7-11 as fluorescent tags for labeling of glycans.

TABLE 2
Photophysical properties of 7-11.

| | | | Emission λₘₐₓ, | Stokes shift, |
|---|---|---|---|---|
| Compound | Solvent | Absorption, nm (ε, M⁻¹cm⁻¹) | nm (Φ_{fl}, %)^{c} | nm |
| 7 | H₂O^{a} | 455 (4700), 300 (27300), 272 (27400) | 611 (3) | 156 |
| | MeOH^{b} | 462 (4100), 352 (5500), 310 (27000), 274 | 584 (17) | 122 |
| | | (28100) | | |
| 8 | H₂O^{a} | 445 (3500), 301 (23600), 270 (26250) | 603 (5) | 158 |
| | MeOH^{b} | 450 (3500), 350 (4500), 306 (29300), 270 | 561 (25) | 111 |
| | | (24000) | | |
| 9 | H₂O^{a} | 455 (5500), 303 (31900), 273 (28700) | 612 (2) | 157 |
| | MeOH^{b} | 459 (5200), 355 (5900), 310 (35500), 275 | 579 (22) | 120 |
| | | (31800) | | |
| 10 | H₂O^{a} | 457 (4700), 303 (27300), 273 (25000) | 615 (2) | 158 |
| | MeOH^{b} | 463 (4700), 354 (5800), 310 (30700), 275 | 582 (19) | 119 |
| | | (30100) | | |
| 11 | H₂O^{a} | 460 (6400), 303 (33700), 275 (30000) | 626 (3) | 166 |
| | MeOH^{b} | 476 (5800), 356 (5800), 313 (35000), 275 | 587 (25) | 111 |
| | | (30800) | | |

^{a}25 mM HEPES pH 8.0;
^{b}MeOH with 0.1% TEA;
^{c}absolute values of the fluorescence quantum yields.

Synthesis of Glucose Conjugates

To study the reaction of the dyes (6-7, 9-11) with glycans under conditions of reductive amination, the labeling of glucose was explored (FIG. 16). A clean conversion to the products (6-7-Glc and 9-11-Glc) was observed (>80% HPLC area). The glucose conjugates were isolated and characterized by ¹H NMR, HRMS, UV-Vis and fluorescence spectroscopy.

Spectroscopic Properties of Glucose Conjugates

The photophysical properties (absorption/emission maxima and quantum yields) of glucose conjugates are summarized in Table 3. In aqueous buffer, their absorption maxima are found at about 470 nm and provide a good match to 488 nm laser. They conjugates with glucose emit orange-red light (with emission maxima at 610-630 nm). The absolute values of the fluorescence quantum yields are 3-4%. Conjugation with glucose results in bathochromic and/or bathofluoric shifts. For example, the absorption and emission maxima of glycosylated sulfone 7-Glc are red-shifted by 18 nm and 12 nm, respectively (in aqueous solutions). In the case of sulfonamides (9-Glc to 11-Glc), glycosylation does not shift the emission maximum to the red region, but results in a bathochromic shift of 11-13 nm. The observed bathochromic shifts can be explained by the stronger electron-donor effect of N-alkyl groups vs. the primary amino group (Table 1). The orange-red emission of the acridine dyes conjugated with glucose is a remarkable feature, as their glycan conjugates (with emission maxima well beyond 600 nm) would have no-cross-talk with the APTS detection window of the standard DNA sequencing equipment (due to negligible emission at 520 nm). Acridines 7 and 9, as stable and more synthetically available compounds, were chosen for further development (phosphorylation).

TABLE 3
Photophysical properties of glucose conjugates 6-Glc to 7-Glc and 9-Glc to 11-Glc.

| | | | Emission λₘₐₓ, | Stokes shift, |
|---|---|---|---|---|
| Compound | Solvent | Absorption, nm (ε, M⁻¹cm⁻¹) | nm (Φ_{fl}, %)^{c} | nm |
| 6-Glc | H₂O^{a} | 471 (5000), 317 (31100), 277 (26100) | 611 (3) | 140 |
| | MeOH^{b} | 480 (4700), 363 (8800), 326 (38100), 283 (29400) | 586 (20) | 106 |
| 7-Glc | H₂O^{a} | 473, 314, 276 | 623 (3) | 150 |
| | MeOH^{b} | 481, 360, 324, 280 | 590 (26) | 109 |
| 9-Glc | H₂O^{a} | 466, 314, 276 | 612 (4) | 146 |
| | MeOH^{b} | 467, 358, 320, 280 | 580 (24) | 113 |
| 10-Glc | H₂O^{a} | 470, 315,276 | 615 (3) | 145 |
| | MeOH^{b} | 471,358,321,280 | 585 (22) | 114 |
| 11-Glc | H₂O^{a} | 473, 314, 276 | 627 (3) | 154 |
| | MeOH^{b} | 482, 361, 324, 279 | 589 (25) | 107 |

^{a}25 mM HEPES pH 8.0;
^{b}MeOH with 0.1% TEA;
^{c}absolute values of the fluorescence quantum yields.

Synthesis and Properties of Phosphorylated Acridines

The residues with terminal hydroxyl groups, in particular 3-hydroxypropyl substituents, enable further modifications of dyes 6-11. For example, decoration with a primary phosphate group increases polarity, makes the dye hydrophilic and water-soluble, and brings a double negative charge (q=−2) in the range of pH (8.0-8.3) recommended for electrophoresis. At pH>8, fluorescent dyes with primary phosphate groups exist in fully ionized forms and move in electric field as sharp single bands.

The phosphorylated analog of diol 7 was prepared according to the route given in FIG. 17. Phosphorylation of diol 22 (for preparation, see FIG. 13) was performed using the general protocol for phosphorylation of alcohols introduced by Haché et al. (B. Haché, L. Brett, S. Shakya, Tetrahedron Lett. 2011, 52, 3625-3629). For one hydroxyl group, it was mandatory to use 3 equiv. of 1H-tetrazole and 2 equiv. of a phosphoramidite reagent. These conditions provided complete conversion of diol 22 to the intermediate bis-phosphite (formation monitored by HPLC), which was oxidized in the reaction mixture with aqueous hydrogen peroxide to alkylphosphate 33 (isolated in 41% yield). Catalytic reduction of the nitro derivative 33 led to amine 34. Both tert-butyl groups in compound 34 were removed upon treatment with 5% TFA in DCM, and the target diphosphate 12 was isolated in a good yield. The final purification (reductive amination of glycans requires highly pure dyes) was achieved by reverse phase chromatography (30 μm, C18AQ, Interchim) using an aqueous buffer (10 mM triethylammonium acetate, pH 7).

In a similar fashion, acridine 30 with N,N-di(2-hydroxyethyl)sulfonamido group (see FIG. 13) was transformed into triphosphate 13 via a three-step route including phosphorylation, reduction and deprotection reactions (FIG. 18). The inventors observed that 7-aminoacridone-2-sulfonamide 36 was formed as a side product in the course of oxidation of phosphite intermediate (not shown) with peroxide. Therefore, the inventors also synthesized acridone diphosphate 39 for spectral comparison.

To prove the applicability of phosphorylated acridines 12 and 13 as new reagents and fluorescent tags for reductive amination and electrophoretic detection of glycans, the inventors prepared their conjugates with glucose, maltotriose and maltoheptaose (FIG. 19, see Experimental Section for details). The inventors used APTS as a reference dye and prepared its conjugates with the same sugars. Compound VBDP was used as a dye similar to compound 12. Both of them have four negative charges and a mass difference of only 2 Da. Spectral properties of the dyes 12, 13, VBDP, their conjugates with glucose, and acridone 39 are collected in Table 4.

TABLE 4
Photophysical properties of the negatively charged dyes and their conjugates with
glucose

| | | | Emission λₘₐₓ, | Stokes shift, |
|---|---|---|---|---|
| Compound | Solvent | Absorption, nm (ε, M⁻¹cm⁻¹) | nm (Φ_{fl}, %)^{c} | nm |
| 12 | H₂O^{a} | 455 (5100), 301 (27400), 272 (24800) | 629 (1) | 174 |
| | MeOH^{b} | 468 (4300), 352 (5500), 312 (26400), 275 | 590 (15) | 122 |
| | | (25900) | | |
| 12-Glc | H₂O^{a} | 469 (4000), 309 (21300), 274 (21300) | 627 (2) | 158 |
| | MeOH^{b} | 483 (3100), 323, (19700), 279 (16600) | 589 (15) | 106 |
| 13 | H₂O^{a} | 455 (5500), 303 (30400), 273 (25800) | 625 (2) | 170 |
| | MeOH^{b} | 470 (4800), 352 (5400), 312 (30300), 276 | 586 (11) | 116 |
| | | (26000) | | |
| 13-Glc | H₂O^{a} | 466,313,275 | 626 (2) | 160 |
| | MeOH^{b} | 480, 322, 279 | 583 (14) | 103 |
| 39 | H₂O^{a} | 422 (3900), 295 (29900), 261 (26200) | 583 (7) | 161 |
| | MeOH^{b} | 428 (3500), 298 (27000) | 562 (20) | 134 |
| VBDP | H₂O^{a} | 431 (5200), 299 (32900), 263 (32900) | 593 (4) | 162 |
| | MeOH^{b} | 435 (4900), 302 (29200) | 575 (15) | 140 |
| VBDP-GIc | H₂O^{a} | 439, 306 | 597 (4) | 158 |
| | MeOH^{b} | 448,310 | 583 (12) | 135 |

^{a}25 mM HEPES pH 8.0;
^{b}MeOH with 0.1% TEA;
^{c}absolute values of the fluorescence quantum yields.

FIG. 1 shows the absorption and emission (2=450 nm) spectra of compounds 13 and 13-Glc in HEPES (25 mM, pH 8).

The structures (FIGS. 17-18), reactivity towards glucose (FIG. 18) and spectral properties (Table 4, FIG. 1) demonstrate that the doubly and triply phosphorylated acridines 12 and 13 have the properties of the fluorescent tags defined above. Additionally, it was necessary to estimate the mobilities of free dyes and their conjugates in electric field. FIG. 2 shows the migration pattern of free dyes premixed with their glycoconjugates. Relative mobility units (RMU) for individual spots were calculated (Table 5). We assumed that the electrophoretic mobilities of the larger labelled oligosaccharides (G3, G7) are inversely proportional to their mass-to-charge ratio (m/z). Glycoconjugates of diphosphate dye 12 migrate slower than the corresponding derivatives prepared from VBDP dye. This indicates that the absolute value of the net charge is smaller for dye 12 (−3) than for dye VBDP (−4) at pH 8.3. The pKₐ values of the structurally similar acridines (A. Albert, R. Goldacre, J. Chem. Soc. 1946, 706) indicate that acridine moiety may be protonated at pH 8.3. If we assume that it is indeed true for dye 12 and other 9-aminoacridines, then triphosphate dye 13 and its conjugates with “neutral” glycans must have 5 negative charges. As expected, the larger conjugates of triphosphate dye 13 move faster than the structurally similar derivatives of APTS and VBDP, which corresponds to the presence of five negative charges. However, due to larger hydrodynamic radius, the free dye 13 moves slower than APTS and VBDP.

FIG. 2 shows the gel electrophoresis results (migration from “north” to “south”, pH 8.3). Lanes I, III and VII are empty. Reading from left to right, the following samples were loaded on each lane (from bottom to top): lane II, APTS, and its conjugates with glucose, maltotriose, maltoheptaose; lane IV, VBDP, and its conjugates with glucose, maltotriose, maltoheptaose; lane V, compound 12, and its conjugates with glucose, maltotriose, maltoheptaose; lane VI, compound 13, and its conjugates with glucose, maltotriose, maltoheptaose. Bands were detected by emission (excitation at 365 nm). Impurities in maltoheptaose give additional (weak) bands. Asterisks (*) indicates bands of interest.

TABLE 5
Gel electrophoresis results from FIG. 2
reported as relative mobility units (RMU)

| Entry | Name (charge) | Mol mass | m/z | RMU^{a} |
|---|---|---|---|---|
| 1 | APTS (-3) | 457 | 152 | 1.00 |
| 2 | -glucose | 621 | 207 | 0.80 |
| 3 | -maltotriose | 945 | 315 | 0.67 |
| 4 | -maltoheptaose | 1593 | 531 | 0.47 |
| 5 | VBDP (-4) | 551 | 138 | 0.94 |
| 6 | -glucose | 715 | 179 | 0.76 |
| 7 | -maltotriose | 1039 | 260 | 0.66 |
| 8 | -maltoheptaose | 1687 | 422 | 0.48 |
| 9 | Dye 12 (-3)^{b} | 549 | 183 | 0.83 |
| 10 | -glucose | 713 | 238 | 0.71 |
| 11 | -maltotriose | 1037 | 346 | 0.62 |
| 12 | -maltoheptaose | 1685 | 562 | 0.45 |
| 13 | Dye 13 (-5)^{b} | 674 | 135 | 0.92 |
| 14 | -glucose | 838 | 168 | 0.78 |
| 15 | -maltotriose | 1162 | 232 | 0.68 |
| 16 | -maltoheptaose | 1810 | 362 | 0.51 |

^{a}Relative mobility units (RMU) were calculated from the cathodal edge of APTS band in the gel.
^{b}Assuming protonation of acridine moiety at pH 8.3.

Compound 13 was tested with a partially hydrolyzed dextran ladder to demonstrate its feasibility in complex carbohydrate analysis (FIG. 3). The inventors used APTS as a reference dye. Images were collected with Amersham Imager 600 with excitation at 460 nm using Cy2 (515 to 535 nm) or Cy3 (560 to 580 nm) filters. Importantly, we observed no emission from conjugates of compound 13 in APTS detection window (515 to 535 nm). Therefore, phosphorylated aminoacridine dyes of the present application and, in particular, compound 13 may be applicable as labeling reagents for producing new internal standards required for calibration of the retention times in CE with LIF detection. The shorter distances between the bands of carbohydrates labeled with dye 13 (FIG. 3) and other doubly phosphorylated 9-aminoacridines are advantageous, as this phenomenon enables higher precision in pinpointing the analyte peaks.

FIG. 3. Gel electrophoresis results (migration from “north” to “south”, pH 8) observed using an Amersham Imager 600 (excitation at 460 nm, Cy2/Cy3 filter overlay). The following samples were loaded on each lane (from bottom to top): lane I, 2 nmol of APTS-G; lane II, 2 nmol of APTS-G7; lane III, 5 nmol of APTS labelled dextran ladder; lane IV, 40 nmol of dextran ladder labelled with compound 13; lane V, 10 nmol of 13-G7; lane VI, 10 nmol of 13-G.

General synthesis and characterization of the novel 1-aminopyrene dyes of the invention and precursors

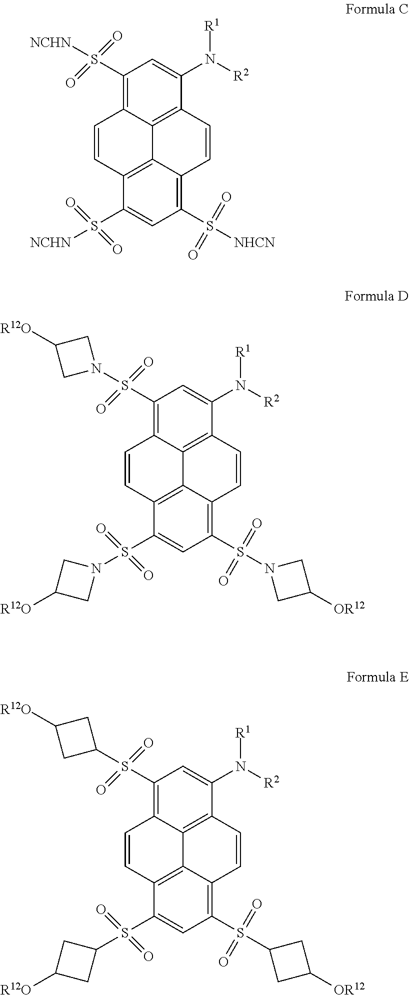

The present invention provides novel aminopyrenes of general formulae C, D and E above as fluorescent dyes decorated with the negatively charged (−1) cyanamidosulfonyl groups (C) or (functionally substituted) hydroxyl groups attached to four-membered rings—azetidinyl (D) or cyclobutyl (E). The dyes emit yellow (C) or orange light (D, E). The hydroxyl groups in structures D and E (if R¹²═H) may be transformed into various functional derivatives, in particular, to primary phosphate residues. As an example of compounds represented by structure C, the inventors obtained 8-amino-1,3,6-tris[(cyanamido)sulfonyl]pyrene (40, FIG. 20) and demonstrated its applicability in reductive amination of carbohydrates (FIG. 4). Importantly, conversion of the sulfonic acid groups in APTS into cyanamidosulfonyl residues in dye 40 results in red shifts of absorption and emission bands (+30 nm). Reductive amination of glucose (and other carbohydrates) with aminopyrene 40 gives conjugates with further bathochromic (+29 nm) and bathofluoric (+13 nm) shifts. The acidity of NH-proton in in the group —SO₂NHCN is high due to the neighboring effect of two strong electron-acceptor groups —SO₂ and CN) and delocalization of the negative charge between two nitrogen atoms (FIG. 20). Therefore, dye 40 and its conjugates move in electric field at pH 8.3 due to the presence of 3 negatively charged —SO₂NCN(—) groups. The gel electrophoresis results are shown in FIG. 4. The samples were applied into three “pockets” on the very top of the plate with a common polyacrylamide gel, and migration (from “north” to “south”) in the buffer solution (pH 8.3) was induced by the applied electrical field. The right (reference) lane shows the spot of the free dye 40 (FIG. 20) with green fluorescence resembling the emission of APTS conjugates. The middle lane contains the bands of APTS-conjugates prepared from the “dextran ladder” (partially hydrolyzed dextran) and APTS dye (for structure, see FIG. 20). These are (from the bottom up): glucose (G), maltose (G₂), maltotriose (G₃), etc. up to G₁₁. The free APTS dye moves too fast and has already left the plate. The left lane shows the free dye 40 (lowest band) and its conjugates obtained from the same “dextran ladder”, as in the middle lane. For APTS conjugates, the extinction coefficient at the maximum (455 nm) is 17000 (Table 6), and the absorption at 488 nm is ca. 35% of the maximal value (R. A. Evangelista, M.-S. Liu, F.-T. A. Chen, Anal. Chem. 1995, 67, 2239-2245). The new dye has a somewhat higher value of the extinction coefficient than APTS, and the absorption maximum of its conjugates (483 nm) is red-shifted by 28 nm (Table 6). Therefore, under excitation with the 488 nm laser, the conjugates of the new dye 40 are at least 3 times “brighter” than APTS derivatives. Due to higher brightness, aminopyrene 40 represents a reagent for reductive amination and fluorescence detection of glycans which provides 3 times higher sensitivity of the detection than APTS. Due to higher m/z ratio (176 vs. 151 of APTS), the conjugates of the new 1-aminopyrene dye 40, and the free dye itself, move somewhat slower than the corresponding APTS derivatives (FIG. 4).

TABLE 6
Spectral properties of aminopyrene dyes and their m/z ratios.

| | | | | Fluor. |
|---|---|---|---|---|
| Dye | Absorption λₘₐₓ, nm | Emission λₘₐₓ, nm | | lifetime |
| (m/z) | (ε, M⁻¹ cm⁻¹) | (Φ_{fl})^{a} | | τ, ns |
| APTS^{b} (151) | 424 (20600) | 500 (0.95) | Solvent | — |
| APTS-G₆ | 455 (17000)^{c} | 511^{c} (0.96)^{d} | H₂O | — |
| 40^{e} (176) | 454 (23900) | 531 (0.93) | H₂O | 5.6 |
| 40-G | 483 | 544 (0.92) | H₂O | 5.3 |
| 51 | 483 (19000) | 534 (0.77) | MeOH | 5.6 |
| 52 (143) | 476^{f} | 543 (0.80)^{f} | aq. TEAB^{f} | 5.9^{f} |
| 52-G | 505 | 565 (0.85) | H₂O | 5.8 |

^{a}absolute values of the fluorescence quantum yields (if not stated otherwise);
^{b}data from ref. [Sharett, Z.; Gamsey, S.; Hirayama, L.; Vilozny, B.; Suri, J. T.; Wessling, R. A.; Singaram; B. Org. Biomol. Chem. 2009, 7, 1461-1470], abs. measured in HO, emission—in aq. phosphate buffer at pH =7.4;
^{c}conjugate with maltohexaose, data from ref. [Evangelista, R. A.; Liu, M.-S.; Chen, F.-T. A. Anal. Chern. 1995, 67, 2239 - 2245];
^{d}an absolute value measured for APTS-glucose conjugate in this study;
^{e}as tris-triethylammonium salt;
^{f}aqueous EtN—H₂CO₃ buffer, pH 8.

Except higher brightness and detection sensitivity, 8-(R-amino)-1,3,6-tri[(cyanamido)sulfonyl]pyrenes (R═H, or carbohydrate residues, as in the left lane of FIG. 4) provide ca. 30 nm red-shift in absorption and emission bands, compared with the corresponding 8-(R-amino)pyrene-1,3,6-trisulfonic acids (APTS, if R═H; or carbohydrate derivatives in the middle lane of FIG. 4). In a broader sense, the present application introduces the (cyanamido)sulfonyl group [—SO₂NHCN] as the negatively charged (at pH>6) electron-acceptor substituent for organic dyes, in particular, for fluorescent dyes having conjugated π-systems. The (cyanamido)sulfonyl group is stronger electron-acceptor than the sulfonic acid, but weaker than the tertiary sulfonamide. This conclusion follows from the data in Table 6, if we assume that the bathochomic and bathofluoric shifts in “push-pull” fluorophores correlate with the difference between the electron-donating (“push”) and electron-attracting (“pull”) properties of the substituents.

FIG. 4 shows gel electrophoresis results (migration from “north” to “south”, pH 8.3). Right (reference) lane: free dye 40 (FIG. 20). Middle lane: APTS-conjugates prepared from the “dextran ladder” (partially hydrolyzed dextran) and APTS dye (FIG. 10); from the bottom up: glucose (G), maltose (G₂), maltotriose (G₃), etc. up to G₁₄. Left lane: dye 40 (lowest band) and its conjugates obtained from the same “dextran ladder” (middle lane). Bands were detected by emission.

In order to increase the bathofluoric shift even further, and improve separation between the emission spectra of APTS and new dyes, the inventors designed 1,6,8-tris[(3-hydroxy-trans cis-cyclobutyl)sulfonyl]-pyrene-3-amines (FIG. 21) and 1,6,8-tris[(3-hydroxyazetidin)sulfonyl]-pyrene-3-amines (FIG. 22). The presence of four-membered rings provides site symmetry in substituents and compact structures with smaller hydrodynamic radius, in comparison with acyclic o-hydroxyalkylsulfonyl and N-(o-hydroxyalkyl)sulfonamido groups possessing higher flexibility and conformational freedom. Alkyl sulfonyl groups and sulfonamido residues have high values of the Hammett σ-constants: σₘ=0.56-0.66, σₚ=0.68-0.77 for SO₂Alkyl and σₘ=0.53, σₚ=0.60 for SO₂NH₂. (a) C. Hansch, A. Leo, R. W. Taft, Chem. Rev. 1991, 91, 165-195; b) D. H. McDaniel, H. C. Brown, J. Org. Chem. 1958, 23, 420-427).

This indicates that they are much more powerful acceptors than (ionized) sulfonic acid residues with σₘ=0.05 and σₚ=0.09 according to H. Zollinger and C. Wittwer, Helv. Chim. Acta. 1953, 36, 1711-1722. The larger values for SO₃⁻ (σₘ=0.30 and σₚ=0.35) are mentioned by C. Hansch, A. Leo, R. W. Taft, Chem. Rev. 1991, 91, 165-195, but these are solely based on a private communication of Viktor Palm.

1-Aminopyrenes of general formulae E possess (functionally substituted) cis- or trans-(3-hydroxycyclobutyl)sulfonyl residues. Their synthesis is exemplified in FIG. 21. At the first step, bromination of 1-(trifluoroacetylamino)pyrene (not shown) leads to tribromide 41 (FIG. 21), an important precursor of the functionally substituted aminopyrenes. We found that trifluoroacetyl residue is a better protecting group for 1-aminopyrene than acetyl (Li, T.; Giasson, R. J. Am. Chem. Soc. 1994, 116, 9890-9893), because it is fairly stable in the presence of acids, but can be easily cleaved under mild basic conditions (e.g, aqueous solution of Na₂CO₃). Palladium-catalyzed cross-coupling [(a) Itoh, T.; Mase, T. Org. Lett. 2004, 6, 4587-4590. (b) Mispelaere-Canivet, C.; Spindler, J.-F.; Perrio, S.; Beslin, P. Tetrahedron, 2005, 61, 5253-5259] of tribromide 41 with trans-3-mercaptocyclobutanol or cis-3-mercaptocyclobutanol (E. Block, J.-A. Lafitte, J. Org. Chem. 1986, 51, 3428-3435) affords triols cis-42 or trans-42 with three pyrenyl 3-hydroxycyclobutyl sulfide residues. Oxidation of cis-42 or trans-42 with concentrated hydrogen peroxide in acetic acid in the presence of sodium tungstate as a catalyst (Schultz, H. S.; Freyermuth, H. B., Buc, S. R. J. Org. Chem. 1963, 28, 1140-1142) leads to trisulfonyl derivatives cis-43 or trans-43. Deprotection of the amino group in compounds cis-43 or trans-43 by heating with aq. NaOH in methanol affords 1-aminopyrenes cis-45 or trans-45. Phosphorylation of dyes cis-45 or trans-45 with excess of POCl₃ in trimethylphosphate occurs on all nucleophilic centers: the three hydroxyl and one aromatic amino group. However, the aromatic amino group in compounds 44 and 45 is only a weak nucleophile. Therefore, the hydrolytic cleavage of phosphate group from phosphorylated amino group occurs already at pH 8-9, and in this way, dyes cis-45 or trans-45 give aminopyrenes cis-44 or trans-44 with three primary phosphate groups. Phosphorylation of compounds cis-43 or trans-43 with excess of POCl₃ in trimethylphosphate occurs only on three hydroxyl groups (the amino group is protected). In this way, aminopyrenes cis-44 or trans-44 can be obtained analogously, but an additional hydrolytic cleavage of trifluoroacetyl group in aqueous Na₂CO₃ is required. 1-Aminopyrenes of general formulae D possess three (functionally substituted) (3-hydroxyazetidino)sulfonyl residues attached to positions 3, 6 and 9. The synthesis is demonstrated in FIG. 22. N-Benzyloxycarbonyl-3-hydroxyazetidine (46) was phosphorylated, and then the Z-group was cleaved to provide amine 48 (with the fully protected phosphate group). In parallel, APTS was converted to tris-sulfonyl chloride 49, an important precursor of the functionally substituted 1,3,6-tri(sulfonamido)-8-aminopyrenes. The reaction between amine 48 and tris-sulfonyl chloride 49 leads to dye 50 with protected phosphate groups. Cleavage of the tert-butyl groups occurs by treating with trifluoroacetic acid in dichloromethane solution followed by evaporation of volatile materials and isolation of the dye 52 via preparative HPLC. The model pyrene dye 51 (with the same chromophore as in phosphorylated dye 52) was prepared from tris-sulfonyl chloride 49 and 3-(tert-butyldimethylsilyloxy)azetidine (not shown in FIG. 22) followed by cleavage of the silyl groups in acetonitrile solution and in the presence of 50-55% aq. HF.

The dyes in Table 6 form two groups. The first group includes compounds with a primary amino group: APTS, 8-amino-1,3,6-tri[(cyanamido)sulfonyl]pyrene (40), sulfonamide 51, and O-phosphorylated sulfonamide 52. The second group includes dyes with N-alkylamino groups: APTS-G₆, 40-G, and 52-G. Compounds of the second group are represented by the products formed in the course of reductive amination (FIG. 6). The absorption maxima of glucose derivatives are red-shifted by about 30 nm, in comparison with the parent dyes, while the emission shows variable bathofluoric shifts of 11, 13 and 22 nm (for APTS, and dyes 40 and 52, respectively). Thus, the Stokes shifts reduced from 67-77 nm to 56-61 nm nm. Importantly, conjugates of the new dyes (40 and 52) absorb and emit at longer wavelengths. This feature is important, as the glycan conjugates of dye 52 have only minimal emission in the APTS detection window. The brightness of a glycan label is termed as a product of the extinction coefficient (at 488 nm, as an excitation wavelength) and the fluorescence quantum yield. The fluorescence quantum yield of APTS-G₆ conjugate (0.96) is somewhat higher than the fluorescence quantum yield of compound 52-G (0.85). However, due to ca. 3 times higher absorbance at 488 nm, the conjugates of the new dyes (40 and 52) provide ca. 3 times higher detection sensitivity than APTS derivatives (under excitation with the 488 nm laser). Due to lower m/z ratio (143 vs. 151 of APTS) and the presence of six negative charges (vs. 3 negative charges in APTS), the conjugates of the new 1-aminopyrene dye 52 move in the electric field much faster than the corresponding APTS derivatives. Therefore, after conjugation with reducing sugars, the phosphorylated pyrene dye 52 with six negative charges can reveal glycans undetectable with APTS due to very long retention times caused by the relatively low net charge (−3) and the limited brightness. In particular, “heavy” glycans require labeling reagents with multiple negative charger (like dye 52).

### BRIEF DESCRIPTION OF THE FIGURES

FIG. 1 shows the absorption and emission (λ_{exc}=450 nm) spectra of compounds 13 and 13-Glc in HEPES (25 mM, pH 8).

FIG. 2 shows gel electrophoresis results for several dyes and their conjugates (migration from “north” to “south”, pH 8.3). Lanes I, III and VII are empty. Reading from left to right, the following samples were loaded on each lane (from bottom to top): lane II, APTS, and its conjugates with glucose, maltotriose, maltoheptaose; lane IV, VBDP, and its conjugates with glucose, maltotriose, maltoheptaose; lane V, compound 12, and its conjugates with glucose, maltotriose, maltoheptaose; lane VI, compound 13, and its conjugates with glucose, maltotriose, maltoheptaose. Bands were detected by emission (excitation at 365 nm).

FIG. 3 shows gel electrophoresis results (migration from “north” to “south”, pH 8) observed using an Amersham Imager 600 (excitation at 460 nm, Cy2/Cy3 filter overlay) for various labelled oligosaccharides. The following samples were loaded on each lane (from bottom to top): lane I, 2 nmol of APTS-G; lane II, 2 nmol of APTS-G7; lane III, 5 nmol of APTS labelled dextran ladder; lane IV, 40 nmol of dextran ladder labelled with compound 13; lane V, 10 nmol of 13-G7; lane VI, 10 nmol of 13-G.

FIG. 4 shows gel electrophoresis results (migration from “north” to “south”, pH 8.3) for various dyes and labelled oligosaccharides. Right (reference) lane: free dye 40 (FIG. 20). Middle lane: APTS-conjugates prepared from the “dextran ladder” (partially hydrolyzed dextran) and APTS dye (FIG. 10); from the bottom up: glucose (G), maltose (G₂), maltotriose (G₃), etc. up to G₁₄. Left lane: dye 40 (lowest band) and its conjugates obtained from the same “dextran ladder” (middle lane). Bands were detected by emission.

FIG. 5 is a bar chart showing increasing information content by passing from genome (whole DNA) over the transcriptome (all RNAs) and the proteome (all proteins) to the glycome (all glycans). [J. E. Turnbull, R. A. Field, Nat. Chem. Biol. 2007, 3, 74-77.]

FIG. 6 shows reductive amination of mono and oligosaccharides. If R³ incorporates the residue of a fluorescent dye, the final product—carbohydrate derivative R²CH₂NHR³—is also fluorescent.

FIG. 7 shows the structure and spectral properties of APTS and its N-alkylated derivatives in aqueous buffers (according to Z. Sharrett, S. Gamsey, L. Hirayama, B. Vilozny, J. T. Suri, R. A. Wessling, B. Singaram, Org. Biomol. Chem. 2009, 7, 1461-1470).

FIG. 8 shows UV-Vis spectra of free APTS (spectrum A) and APTS-maltohexaose (spectrum B). Both were measured as 15 μM solutions in 50 mM phosphate buffer at pH 7.0. The vertical dashed line at 488 nm marks the main wavelength of the argon-ion laser. Adapted from R. A. Evangelista, M.-S. Liu, F.-T. A. Chen, Anal. Chem. 1995, 67, 2239-2245. Copyright (1995) American Chemical Society.

FIG. 9 shows model 7,9-diaminoacridines (6-11) with electron-acceptor cyano, alkylsulfonyl and sulfonamido groups at C-2.

FIG. 10 show negatively charged 7,9-diaminoacridines (12, 13) with primary phosphate groups and reference compounds (APTS and acridone dye VBDP).

FIG. 11 shows a synthesis of 2-bromo-7-nitro-9-phenoxyacridine (18).

FIG. 12 shows synthesis of 2-amino-7-cyano-9-(6-hydroxyhexyl)aminoacridine (6).

FIG. 13 shows synthesis of 7-amino-9-(3-hydroxypropyl)amino-2-[(3-hydroxypropyl)sulfonyl]-acridine 7.

FIG. 14 shows synthesis of acridine-7-sulfonamides 8 and 9 with two amino groups in 2- and 9-positions.

FIG. 15 shows synthesis of acridine-7-sulfonamides 10 and 11.

FIG. 16 shows glucose conjugates prepared from dyes 6-7 and 9-11 via reductive amination.

FIG. 17 shows synthesis of aminoacridine dye 12 decorated with two primary phosphate groups.

FIG. 18 shows synthesis of aminoacridine-triphosphate (13) and acridone-diphosphate (39) dyes.

FIG. 19 shows glycoconjugates prepared via reductive amination from the negatively charged amino-dyes and glucose oligomers.

FIG. 20 shows conversion of APTS dye (trisodium salt) into 8-amino-1,3,6-tri[(cyanamido)sulfonyl]pyrene (40).

FIG. 21 shows triple O-phosphorylated 1,6,8-tris[(3-hydroxy-trans cis-cyclobutyl)sulfonyl]-pyrene-3-amines 44: a) trans- or cis-3-mercaptocyclobutanol (see scheme), DIPEA, Pd₂(dba)₃, XantPhos, DMF, 100° C., overnight; b) aq. H₂O₂, Na₂WO₄, AcOH, r. t., overnight; c) aq. NaOH, MeOH; d) POCl₃, (MeO)₃PO, r. t., then aq. Et₃N*H₂CO₃ buffer (pH 8-9); e) aq. Na2CO3.

FIG. 22 shows synthesis of tris-O-phosphorylated 1-aminopyrene 52: a) di-tert-butyl N,N-diisopropylphosphoramidite, 1H-tetrazole, DMF, r. t., 1 h, then 30% H₂O₂, 0° C.→r. t., 6 h; b) ClSO₃H, 65° C., 3 h; c) H₂, Pd/C, MeOH, r. t., overnight; d) Et₃N, MeCN, r. t., 10 min; e) 3-(tert-butyldimethylsilyloxy)azetidine, Et₃N, MeCN, r. t., 1 h; then 50-55% aq. HF, MeCN, 0° C.→r. t., overnight; f) CF₃CO₂H, DCM, r. t., 1 h, then 1 M aq. Et₃N*H₂CO₃ (pH 8-9), overnight.

FIGS. 23, 24, 25, 26 and 27 show absorption and emission spectra.

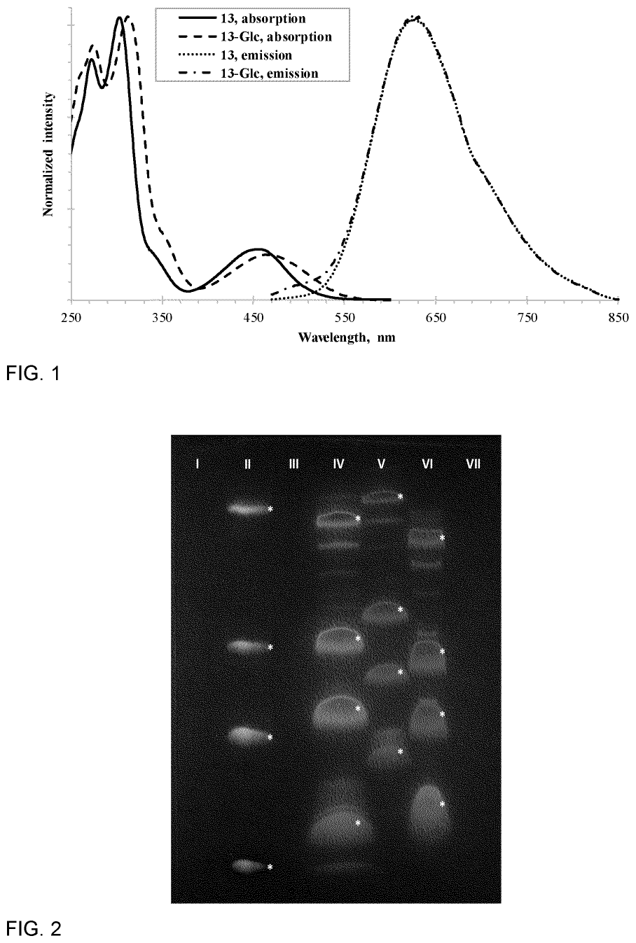

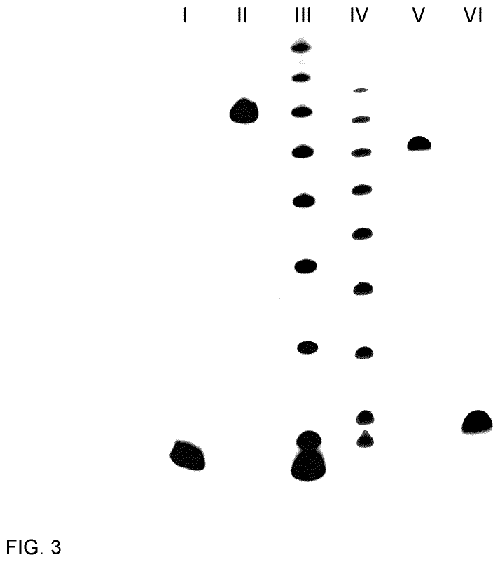

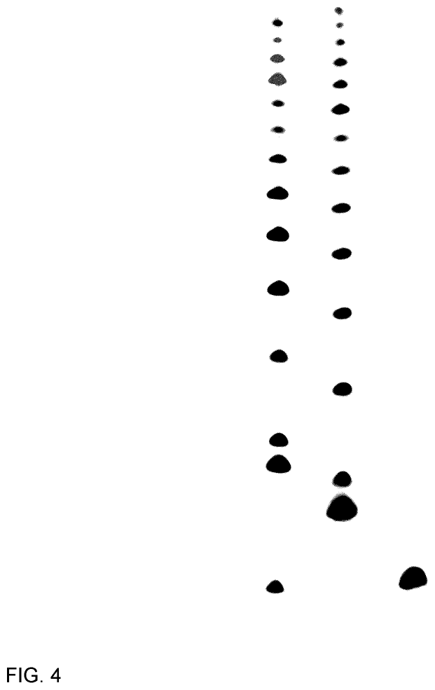

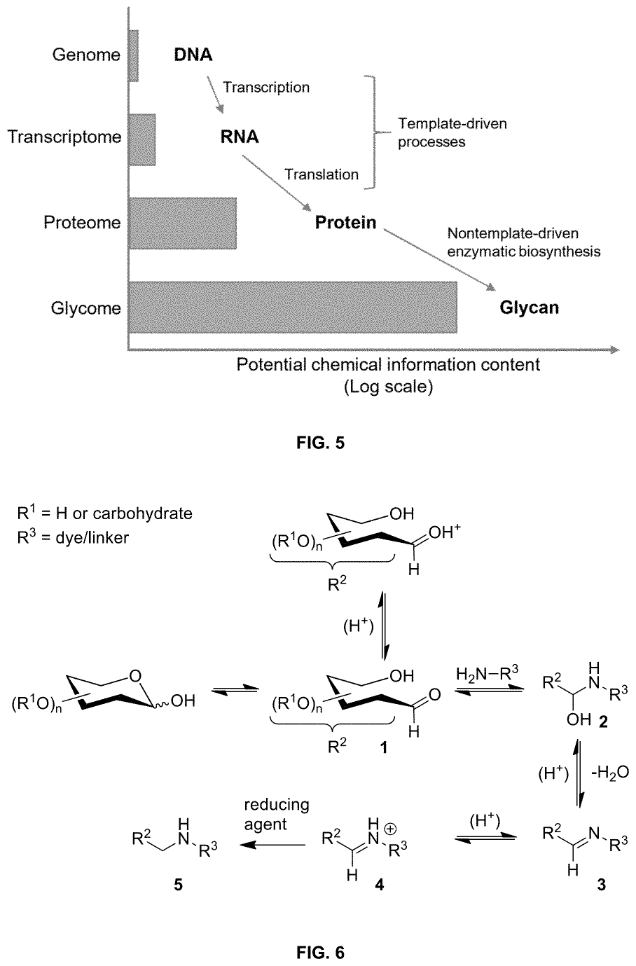

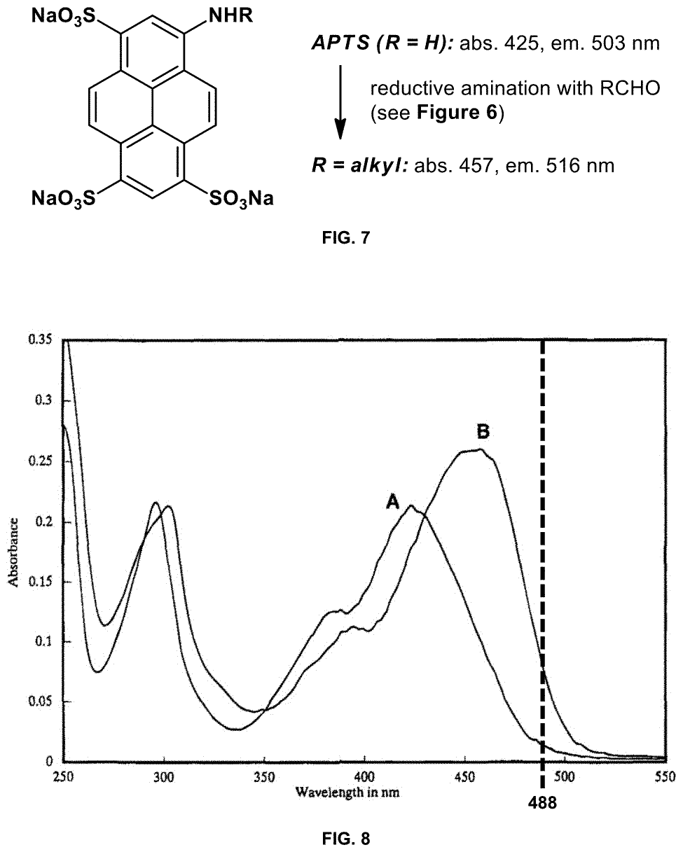

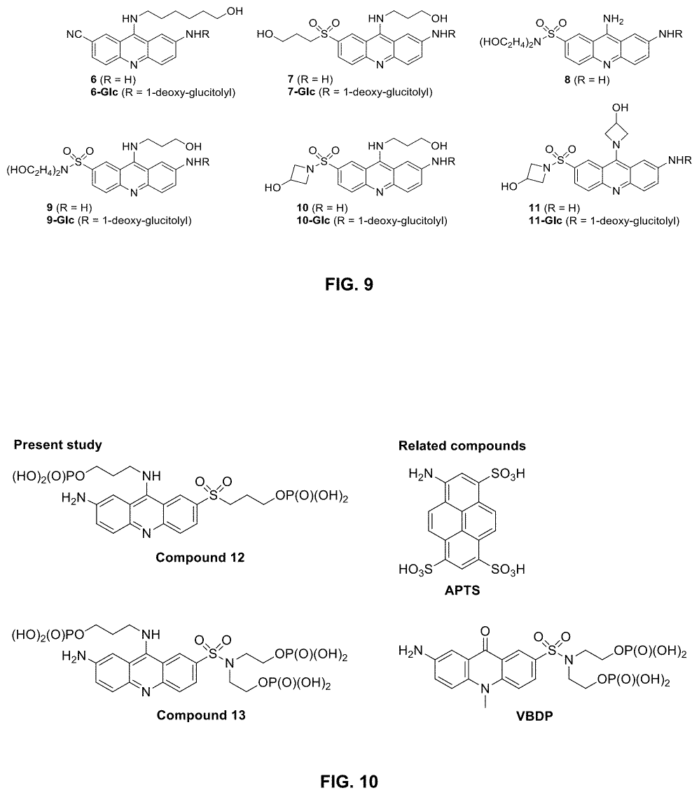

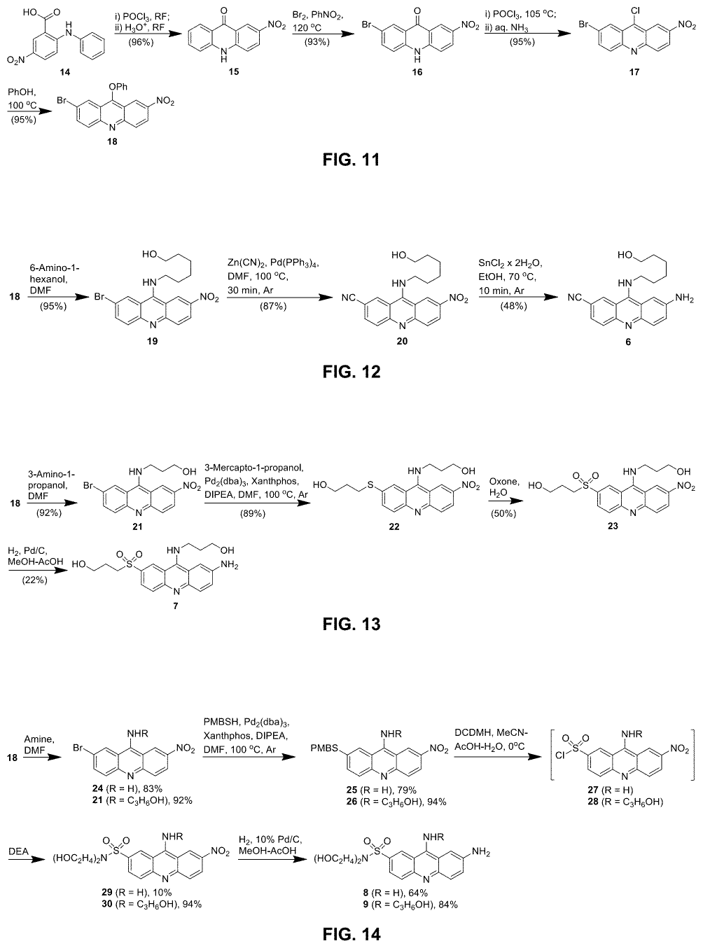

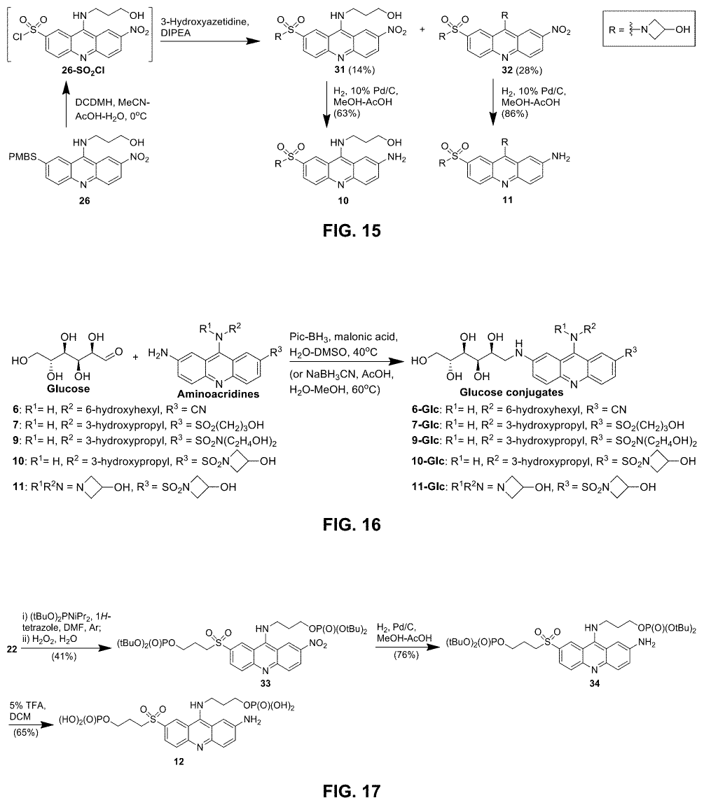

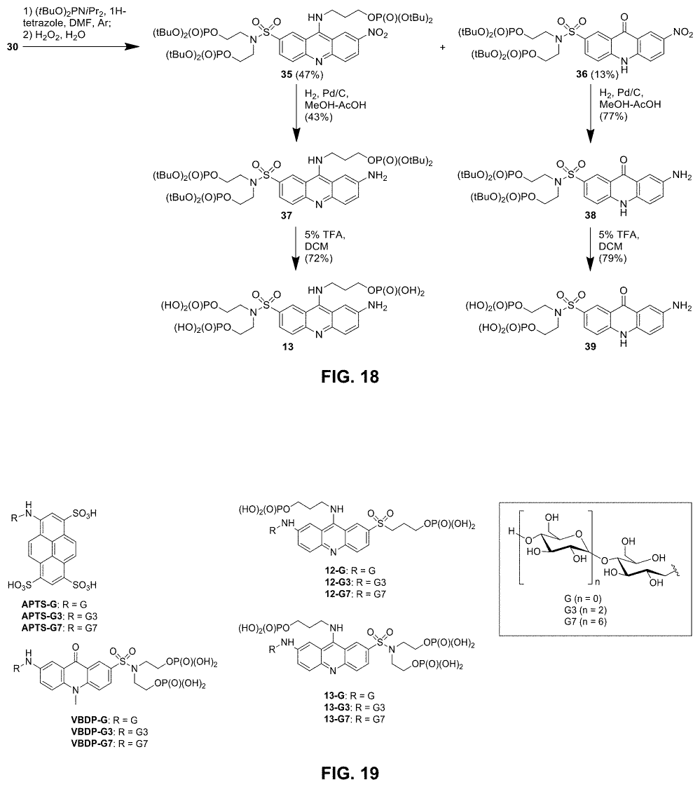

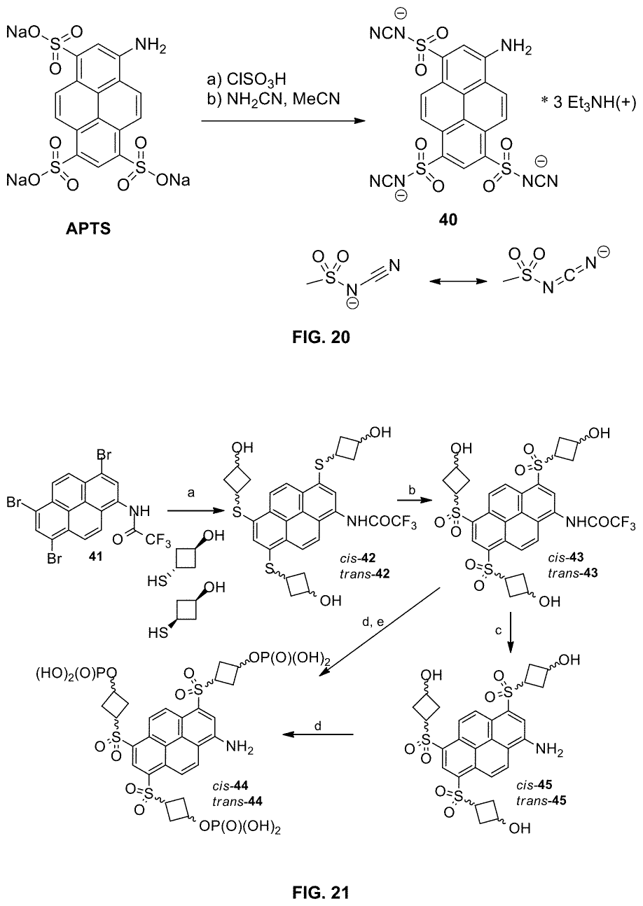

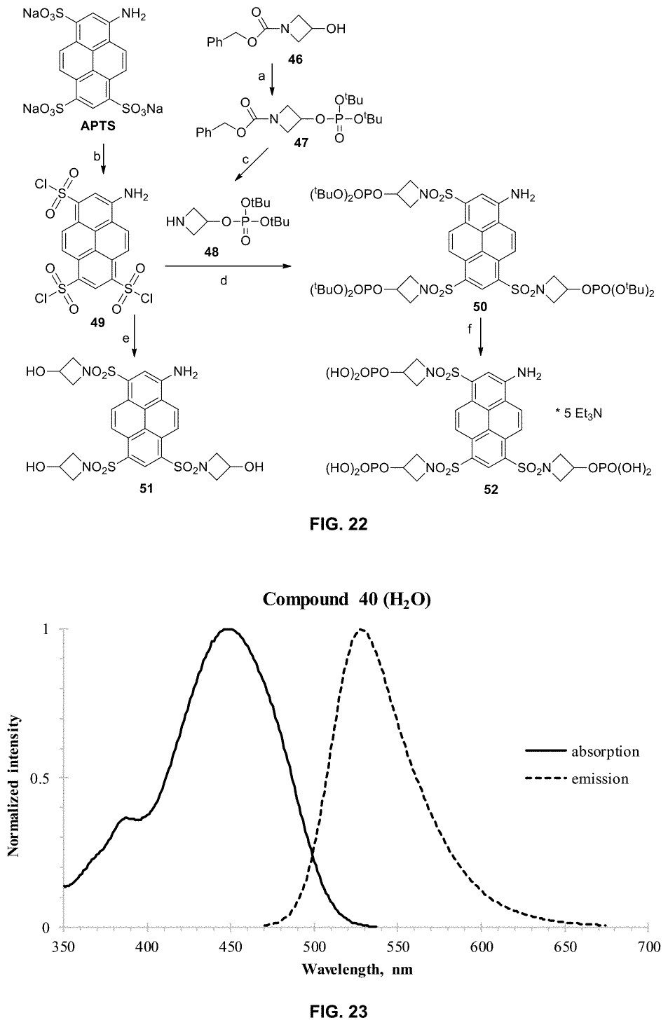

The following Examples illustrate the present invention in more detail.

General Materials and Methods

All commercially available chemicals (Acros, Aldrich, Fluka, Merck, Alfa Aesar, TCI) were used as received. The solvents had the purity “pro analysis”. For photophysical measurements solvents of spectrophotometric grade were used. Anhydrous solvents were stored over molecular sieves. Deuterated solvents were purchased from Deutero GmbH (CDCl₃, CD₃OD, D₂O, DMF-d₇, DMSO-d₆, TFA-d). Reactions at “0° C.” were carried out using an ice-bath.

TLC. Normal phase TLC was performed on silica gel 60 F₂₅₄ (Merck Millipore). Compounds were detected by exposing TLC plates to UV-light (254 or 366 nm).

Analytical HPLC. Analytical HPLC was performed on a Knauer Azura liquid chromatography system with a binary P 6.1 L pump (Article No. EPH35, Knauer), UV diode array detector DAD 6.1 L (Article No. ADC11, Knauer), an injection valve with a 20 μL loop and two electrical switching valves V 2.1 S with 6-port multiposition valve head (Article No. EWA10, Knauer). The UV detection was carried out with double wavelength pattern at wavelength channel 1 of 254 nm and wavelength channel 2 of 350 nm. The column temperature was not standardized, but remained at around 25° C.

Analytical columns: Knauer Eurospher II 100-5 C18, 5 m, 150×4 mm (if not stated otherwise), or Knauer Eurospher II 100-5 C18A, 5 m, 150×4 mm, or Knauer Eurospher II 100-10 C18A, m, 150×4 mm, or Interchim Uptisphere Strategy C18-HQ, 10 μm, 250×4.6 mm.

Flow rate: 1.2 mL/min.

Phase A: water+0.1% v/v trifluoroacetic acid (TFA).

Phase B: MeCN+0.1% v/v TFA.

Method 20-100: 20B (3 min); 20-100B (12 min).

Method 5-50: 5B (3 min); 5-50B (12 min); 50-100B (3 min).

For isolation and purification of phosphorylated dyes, acetonitrile (phase B)—aqueous systems containing 0.05 M of TEAB buffer (phase A, pH=8; self-prepared from 1 M aq. Et₃N and CO₂ gas obtained by evaporation of solid CO₂).

Method TEAB-0-25: OB (3 min); 0-25B (12 min); 25-100B (3 min).

Method TEAB-0-40: OB (3 min); 0-40B (12 min); 40-100B (3 min).

FC. Flash chromatography was performed on an automated Biotage Isolera One or Interchim puriFlash™ flash purification system using the cartridges and solvent gradients indicated in the text.

MS. Mass spectra with ESI Ion source were recorded by J. Bienert (Chemical Facility, Max Planck Institute for Biophysical Chemistry, Göttingen) using a Varian 500 MS (Agilent). High resolution mass spectra were obtained on a Bruker maXis (ESI-QTOF-HRMS) or Bruker Autoflex Speed (MALDI-TOF HRMS) spectrometer by the team of Dr. H. Frauendorf (Facility of Mass Spectrometry, Georg-August-Universitat Gottingen).

NMR. NMR spectra were recorded on an Agilent 400MR DD2 spectrometer. All spectra are referenced to tetrametylsilane as an internal standard (6=0.00 ppm) using the signals of the residual protons of CHCl₃ (7.26 ppm) in CDCl₃, CHD₂OD (3.31 ppm) in CD₃OD, CHD₂COCD₃ (2.05 ppm) in (CD₃)₂CO or DMSO-d₅ in DMSO-d₆. Multiplicities of the signals are described as follows: s=singlet, br. s=broad singlet, d=doublet, t=triplet, q=quartet, m=multiplet. Coupling constants J are given in Hz. For ¹³C-signals, which were revealed by indirect detection by HSQC, only resonances of the carbon atoms linked to H-atoms were recorded.

UV-Vis and fluorescence. Absorption spectra was recorded on a double-beam UV-Vis spectrophotometer (Varian, series 4000). Measurements were performed in 1-cm quartz cells or UV-Vis disposable cuvettes (BRAND semi-micro), and in air-equilibrated solutions at ambient temperature (24-25° C.). Emission spectra and fluorescence quantum yield were obtained on a Quantaurus-QY Absolute PL quantum yield spectrometer C11347 (Quantaurus QY) or on a Cary Eclipse fluorescence spectrometer (Varian). Emission and UV-Vis scan spectra were recorded using following parameters: average time 0.1 s; data interval 1 nm; scan rate 600 nm/min; with base line correction.

Gel electrophoresis. The gel contained 50 mL of 20% of acrylamide, which was made up in the standard TBE buffer (89 mM Tris, 89 mM borate, 2 mM EDTA) containing 7 M urea. Polymerization was catalyzed by the addition of 162.5 μL of a ammoniumpersulfate (APS, 25 wt. % solution in water) and 43.8 μL of N,N,N′,N′-tetramethylethyleendiamine (TEMED). The gels were of the 8- or 17-well format (width 20 cm) with 30 cm well-to-read length and 0.75 mm thickness. The running buffer was 89 mM Tris-borate pH 8.3 containing 2 mM EDTA. Electrophoresis was performed at a constant power of 35 W (Consort EV3330) at ambient temperature with forced air cooling, the front glass plate was equipped with an external aluminium plate. After pre-running the gel for 30 min, the wells were thoroughly rinsed with the TBE buffer, and appropriate volumes (30-50 μL, ca. 50% formamide) of the samples were loaded. Usually one gel lane was skipped between each two samples, to avoid cross-contamination and to ease the lane tracking process. The electrophoresis voltage during separation was 1700-2200 V and the analysis was run until APTS reached the bottom of the gel (1.5-2 h). The fluorescence of APTS- and acridine derivatized carbohydrates was readily resolved in a UV viewing cabinet (254/365 nm) equipped with a digital camera or using Amersham Imager 600.

### Example 1

### Synthesis of Fluorescent Acridone Dyes and their Precursors

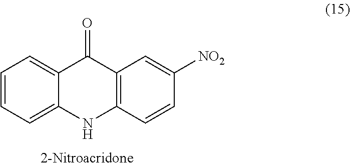 [SMILES: O=c1c2ccccc2[nH]c2ccc([N+](=O)[O-])cc12]

2-Nitroacridone was synthesized according to the literature (P. Graves and J. A. Smith. “Fluorogenic peptides and their method of production.” US 2011/0039289 A1, Mar. 14, 2008). ¹H NMR (400 MHz, DMSO-d₆): δ=12.33 (s, 1H), 8.95 (d, J=2.7 Hz, 1H), 8.45 (dd, J=9.4, 2.7 Hz, 1H), 8.22 (dd, J=8.2, 1.2 Hz, 1H), 7.80 (ddd, J=8.2, 7.0, 1.6 Hz, 1H), 7.65 (d, J=9.4 Hz, 1H), 7.58 (d, J=8.2 Hz, 1H), 7.36 (ddd, J=8.2, 7.0, 0.8 Hz, 1H) ppm.

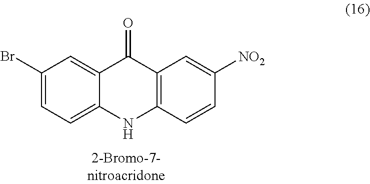 [SMILES: O=c1c2cc(Br)ccc2[nH]c2ccc([N+](=O)[O-])cc12]

2-Bromo-7-nitroacridone was synthesized according to the literature (A. Kliegl, L. Schaible, Chem. Ber. 1957, 90, 60-65.). 1H NMR (400 MHz, DMSO-d₆): δ=12.47 (s, 1H), 8.92 (d, J=2.7 Hz, 1H), 8.46 (dd, J=9.2, 2.7 Hz, 1H), 8.27 (d, J=2.3 Hz, 1H), 7.93 (dd, J=8.6, 2.3 Hz, 1H), 7.65 (d, J=9.2 Hz, 1H), 7.54 (d, J=8.6 Hz, 1H) ppm.

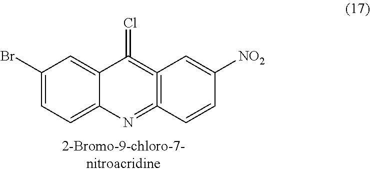

2-Bromo-9-chloro-7-nitroacridine was synthesized according to the literature (K. Singh, G. Singh, Indian J. Pharm. 1952, 14, 47-49.). TLC (SiO₂): R_{f}=0.4 (DCM/MeOH 20:0.05). 1H NMR (400 MHz, CDCl₃): δ=9.41 (d, J=2.7 Hz, 1H), 8.64 (d, J=2.0 Hz, 1H), 8.53 (dd, J=9.4, 2.7 Hz, 1H), 8.33 (d, J=9.4 Hz, 1H), 8.13 (d, J=9.0 Hz, 1H), 7.96 (dd, J=9.0, 2.0 Hz, 1H) ppm.

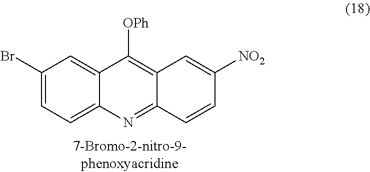 [SMILES: O=[N+]([O-])c1ccc2nc3ccc(Br)cc3c(Oc3ccccc3)c2c1]

A mixture of 7-bromo-9-chloro-2-nitroacridine (76 mg, 225 μmol) and phenol (1 g) was stirred at 100° C. for 1.5 hours under Ar. The reaction mixture was allowed to cool down to rt, diluted with DCM and washed with 0.1 M NaOH to remove phenol. The organic phase was further washed with brine (until pH became neutral), dried (Na₂SO₄), and the solvent was removed in vacuo to give a yellow solid (85 mg, 95%). TLC (SiO₂): R_{f}=0.35 (DCM/MeOH 20:0.05). ¹H NMR (400 MHz, CDCl₃): δ=9.05 (d, J=2.0 Hz, 1H), 8.50 (dd, J=9.4, 2.3 Hz, 1H), 8.34 (dd, J=9.4, 0.8 Hz, 1H), 8.27 (d, J=1.6 Hz, 1H), 8.15 (d, J=9.4 Hz, 1H), 7.92 (dd, J=9.4, 2.3 Hz, 1H), 7.32-7.38 (m, 2H), 7.12-7.19 (m, 1H), 6.86-6.91 (m, 2H) ppm. ¹¹C NMR (101 MHz, CDCl₃): δ=159.2, 157.3, 151.0, 150.9, 145.2, 136.3, 131.9, 131.7, 130.4, 124.9, 123.8, 123.7, 121.6, 121.6, 120.8, 118.9, 115.7 ppm. HRMS: m/z 395.0022 ([M+H]⁺) calculated for C₁₉H₁₂BrN₂O₃+: 395.0026 (Δ1.0 ppm).

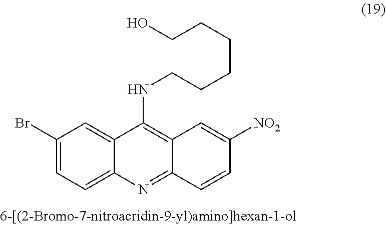 [SMILES: O=[N+]([O-])c1ccc2nc3ccc(Br)cc3c(NCCCCCCO)c2c1]

A mixture of 7-bromo-2-nitro-9-phenoxyacridine (85 mg, 215 μmol) and 6-amino-1-hexanol (50 mg, 430 μmol) in dry DMF (5 mL) was stirred at rt for 1 hour under Ar. The reaction mixture was diluted with DCM (50 mL) and washed with 0.1 M NaOH to remove phenol. The organic phase was further washed with brine (until pH became neutral), dried (Na₂SO₄), and the solvent was removed in vacuo to give a red-orange powder (85 mg, 95%). TLC (SiO₂): R_{f}=0.19 (DCM/MeOH 20:1), R_{f}=0.39 (DCM/MeOH 20:2). HPLC (20-100, Knauer Eurospher II 100-10 C18A): t_{R}=8.7 min. ESI-MS: m/z 418.2 [M+H]⁺. ¹H NMR (400 MHz, MeOD-d₃ with TFA-d): δ=9.22-9.72 (m, 1H), 8.60-8.84 (m, 1H), 8.71 (dd, J=9.4, 2.3 Hz, 1H), 8.15 (dd, J=9.0, 2.0 Hz, 1H), 7.91 (d, J=9.4 Hz, 1H), 7.77 (d, J=9.0 Hz, 1H), 4.14-4.20 (m, 2H), 3.56-3.61 (m, 2H), 1.99-2.18 (m, 2H), 1.57-1.67 (m, 4H), 1.47-1.57 (m, 2H) ppm.

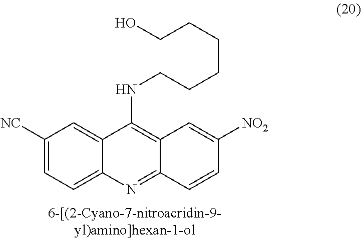 [SMILES: N#Cc1ccc2nc3ccc([N+](=O)[O-])cc3c(NCCCCCCO)c2c1]

Compound 19 (127 mg, 304 μmol), Zn(CN)₂ (71 mg, 608 μmol) and Pd(PPh₃)₄ (35 mg, 30 μmol) were placed in a heat dried Schlenk tube filled with Ar, which was evacuated-backfilled with Ar (3×). DMF (2.5 mL) was added and the reaction mixture was immediately heated up to 100° C. After 30 min of stirring at the same temperature, the reaction mixture was allowed to cool down to rt, diluted with DCM, washed with brine, dried (Na₂SO₄) and concentrated in vacuo. The residue was purified by FC (SiO₂, RediSep Rf 24 g cartridge, dry-load, DCM/MeOH with 0.5-5% MeOH gradient) to give a red solid (96 mg, 87%). TLC (SiO₂): R_{f}=0.42 (DCM/MeOH 10:1). HPLC (20-100, Knauer Eurospher II 100-10 C18A): t_{R}=7.0 min. ESI-MS: m/z 365.2 [M+H]⁺. ¹H NMR (400 MHz, MeOD-d₃ with TFA-d): δ=9.26-9.72 (m, 1H), 8.81-9.08 (m, 1H), 8.75 (dd, J=9.4, 2.0 Hz, 1H), 8.23 (dd, J=9.0, 1.6 Hz, 1H), 7.85-7.98 (m, 2H), 4.16-4.26 (m, 2H), 3.54-3.63 (m, 2H), 1.98-2.22 (m, 2H), 1.57-1.72 (m, 4H), 1.48-1.56 (m, 2H) ppm.

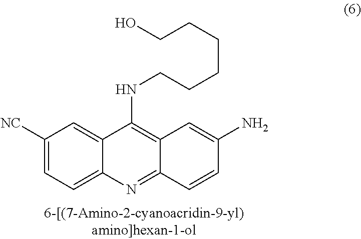 [SMILES: N#Cc1ccc2nc3ccc(N)cc3c(NCCCCCCO)c2c1]

Compound 20 (64 mg, 176 μmol) and SnCl₂×2 H₂O (199 mg, 880 μmol) were placed in a Schlenk flask, which was evacuated-backfilled with Ar. Absolute EtOH (0.5 mL) was added and the reaction mixture was immediately heated up to 70° C. After 15 min of stirring at the same temperature, the reaction mixture was allowed to cool down to rt, diluted with 18 mL of 0.1% aq. TFA and purified by FC (C18, 30C18AQ-F0025 cartridge, H₂O/ACN (with 0.1% TFA) with 0-50% ACN gradient). Good fractions were pooled, concentrated in vacuo, and the excess of TFA was removed by filtration through C18 column using H₂O-ACN as eluents. The resulting orange filtrate was diluted with H₂O and lyophilized to give a red-orange powder (48 mg, 48%, TFA salt). HPLC (Knauer Eurospher II 100-10 C18A): t_{R}=4.8 min (20-100); t_{R}=10.7 min (5-50). ESI-MS: m/z 335.4 [M+H]⁺. HRMS: m/z 335.1867 ([M+H]⁺) calculated for C₂₀H₂₃N₄O⁺: 335.1866 (Δ0.3 ppm). ¹H NMR (400 MHz, D₂O): δ=8.29 (s, 1H), 7.82 (br d, J=8.6 Hz, 1H), 7.47 (d, J=9.0 Hz, 1H), 7.32-7.42 (m, 2H), 7.10 (s, 1H), 3.81 (br dd, J=7.4, 7.0 Hz, 2H), 3.56 (dd, J=6.7, 6.3 Hz, 2H), 1.69-1.89 (m, 2H), 1.46-1.60 (m, 2H), 1.28-1.45 (m, 4H) ppm. The following signals were visible in the ¹³C NMR (100 MHz, D₂O/ACN-d₃, HSQC) spectra of 6: 6=134.3 (CH), 124.8, 120.1 (CH), 119.7 (CH), 118.7, 61.6 (CH₂), 48.9 (CH₂), 31.3 (CH₂), 29.0 (CH₂), 25.8 (CH₂), 24.8 (CH₂) ppm. ¹⁹F NMR (376 MHz, D₂O): δ=−75.53 ppm.

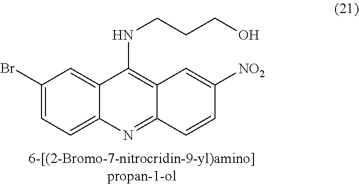 [SMILES: O=[N+]([O-])c1ccc2nc3ccc(Br)cc3c(NCCCO)c2c1]

A mixture of 7-bromo-2-nitro-9-phenoxyacridine (500 mg, 1.27 mmol) and 3-amino-1-propanol (191 mg, 2.54 mmol) in dry DMF (15 mL) was stirred at rt for 1 hour under Ar. The reaction mixture was diluted with DCM-MeOH (10:1, 500 mL) and washed with 0.1 M NaOH to remove phenol. MeOH was added to the organic phase whenever precipitates were formed. The organic phase was further washed with brine (until pH became neutral), dried (Na₂SO₄), and the solvent was removed in vacuo to give a red powder (440 mg, 92%). TLC (SiO₂): R_{f}=0.18 (DCM/MeOH 20:1); R_{f}=0.46 (DCM/MeOH 10:1); R_{f}=0.55 (DCM/MeOH/H₂O 90:10:1). HPLC (20-100): t_{R}=7.2 min; λₘₐₓ=442, 423, 363, 313, 271, 251 nm. ESI-MS: m/z 376.0 [M+H]⁺. HRMS: m/z 376.0291 ([M+H]⁺) calculated for C₁₆H₁₅BrN₃O₃: 376.0291 (Δ0.0 ppm). ¹H NMR (400 MHz, MeOD-d₃ with TFA-d): δ=9.45 (d, J=2.0 Hz, 1H), 8.64-8.75 (m, 2H), 8.13 (dd, J=9.0, 2.0 Hz, 1H), 7.92 (d, J=9.0 Hz, 1H), 7.78 (d, J=9.0 Hz, 1H), 4.39 (t, J=6.6 Hz, 2H), 3.91 (t, J=5.9, 5.5 Hz, 2H), 2.23 (quin., J=6.6, 5.9, 5.5 Hz, 2H) ppm.

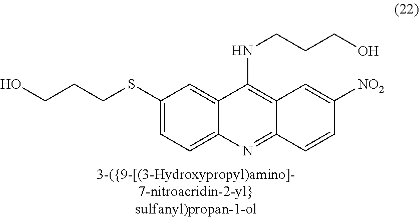 [SMILES: O=[N+]([O-])c1ccc2nc3ccc(SCCCO)cc3c(NCCCO)c2c1]

Compound 21 (50 mg, 133 μmol), Pd₂(dba)₃ (7.4 mg, 8 μmol) and Xantphos (8.9 mg, 15 μmol) were placed in a heat dried Schlenk tube filled with Ar, which was evacuated-backfilled with Ar (3×). DMF (1.3 mL), DIPEA (46 μL, 266 μmol) and 3-mercapto-1-propanol (15 mg, 160 μmol) were added and the reaction mixture was immediately heated up to 100° C. After 1 h of stirring at the same temperature, the reaction mixture was allowed to cool down to rt, diluted with DCM-MeOH (10:1), washed with 10% aq. Na₂SO₃— aq. sat. NaHCO₃ (1:1) and brine, dried (Na₂SO₄) and concentrated in vacuo. The residue was purified by FC (SiO₂, RediSep R_{f} 24 g cartridge, dry-load, DCM/MeOH with 0-15% MeOH gradient) to give a dark-red solid (46 mg, 89%). TLC (SiO₂): R_{f}=0.24 (DCM/MeOH 10:1); R_{f}=0.3 (DCM/MeOH/H₂O 90:10:1). HPLC: t_{R}=7.0 min (20-100); t_{R}=12.1 min (5-50); λₘₐₓ=442, 345, 273 nm. ESI-MS: m/z 388.0 [M+H]⁺. HRMS: m/z 388.1327 ([M+H]⁺) calculated for C₁₉H₂₂N₃O₄S⁺: 388.1326 (Δ0.3 ppm). ¹H NMR (400 MHz, CDCl₃/MeOD-d₃ 7:3): δ=9.29 (d, J=2.3 Hz, 1H), 8.26 (dd, J=9.4, 2.3 Hz, 1H), 7.99 (d, J=2.0 Hz, 1H), 7.80 (d, J=9.4 Hz, 1H), 7.76 (d, J=9.0 Hz, 1H), 7.61 (dd, J=9.0, 2.0 Hz, 1H), 4.19 (t, J=6.3, 5.9 Hz, 2H), 3.93 (t, J=5.5, 5.1 Hz, 2H), 3.69 (t, J=5.9 Hz, 2H), 3.10 (t, J=7.4, 7.0 Hz, 2H), 2.04-2.11 (m, 2H), 1.82-1.97 (m, 2H) ppm.

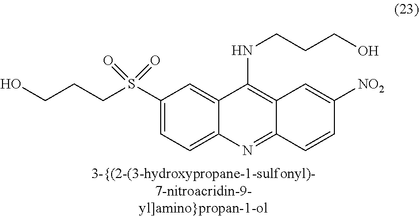 [SMILES: O=[N+]([O-])c1ccc2nc3ccc(S(=O)(=O)CCCO)cc3c(NCCCO)c2c1]

Compound 22 (110 mg, 284 μmol) was suspended in H₂O (6 mL). Oxone (166 mg, 270 μmol) was added in small portions. The suspension changed from red to yellow color. After 30 min of stirring at rt, the reaction mixture was diluted with 10% aq. Na₂SO₃— aq. sat. NaHCO₃ (1:1), and the product was extracted into EtOAc (containing small amounts of MeOH). The organic phase was washed with brine, dried (Na₂SO₄) and concentrated in vacuo. The residue was purified by FC (SiO₂, RediSep R_{f} 24 g cartridge, DCM/MeOH/H₂O 90:10:1) to give a light-orange solid (60 mg, 50%). TLC (SiO₂): R_{f}=0.3 (DCM/MeOH/H₂O 90:10:1); R_{f}=0.1 (DCM/MeOH 20:1). HPLC: t_{R}=10.3 min (5-50); λₘₐₓ=426, 408, 357, 303, 266 nm. ESI-MS: m/z 420.2 [M+H]⁺. HRMS: m/z 420.1227 ([M+H]⁺) calculated for C₁₉H₂₂N₃O₆S⁺: 420.1224 (Δ0.7 ppm). ¹H NMR (400 MHz, MeOD-d₃ with TFA-d): δ=9.39-9.57 (m, 1H), 9.04 (d, J=1.6 Hz, 1H), 8.76 (dd, J=9.4, 1.6 Hz, 1H), 8.43 (dd, J=9.0, 1.6 Hz, 1H), 7.99 (d, J=9.0 Hz, 1H), 7.95 (d, J=9.4 Hz, 1H), 4.33-4.53 (m, 2H), 3.86-3.99 (m, 2H), 3.63 (t, J=6.3 Hz, 2H), 3.39-3.47 (m, 2H), 2.26 (quin., J=6.7, 6.3, 5.9, 5.5 Hz, 2H), 1.88-1.98 (m, 2H) ppm.

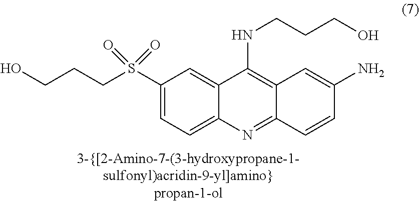 [SMILES: Nc1ccc2nc3ccc(S(=O)(=O)CCCO)cc3c(NCCCO)c2c1]

Compound 23 (15 mg, 36 μmol) was dissolved in MeOH (10 mL) and AcOH (10 μL) under Ar. A 10% Pd/C catalyst (7 mg) was added, and hydrogen atmosphere was applied. After 1 hour of stirring at rt, orange fluorescent solution was filtered through a pad of Celite, and concentrated in vacuo. The residue was dissolved in 10 mL of H₂O and purified by FC (C18, 30C18AQ-F0025 cartridge, ACN/10 mM TEAAc pH 7.0) to give a red solid (3.4 mg, 22%, AcOH salt). HPLC: t_{R}=9.0 min (5-50); λₘₐₓ=428, 408, 301, 273, 227 nm. HRMS: m/z 390.1488 ([M+H]⁺) calculated for C₁₉H₂₄N₃O₄S⁺: 390.1482 (Δ1.5 ppm). ¹H NMR (400 MHz, D₂O/MeCN-d₃ 6:1): δ=8.69 (d, J=2.0 Hz, 1H), 8.07 (dd, J=9.0, 1.6 Hz, 1H), 7.78 (d, J=9.0 Hz, 1H), 7.56 (d, J=9.0 Hz, 1H), 7.45 (dd, J=9.0, 2.0 Hz, 1H), 7.24 (d, J=1.6 Hz, 1H), 4.09 (t, J=7.0 Hz, 2H), 3.91 (t, J=5.9 Hz, 2H), 3.73 (t, J=6.3 Hz, 2H), 3.51-3.63 (m, 2H), 2.17 (quin, J=6.3 Hz, 2H), 2.00-2.07 (m, 2H), 1.99 (s, AcOH) ppm.

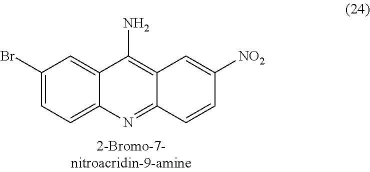 [SMILES: Nc1c2cc(Br)ccc2nc2ccc([N+](=O)[O-])cc12]

A mixture of 2-bromo-7-nitro-9-phenoxyacridine (100 mg, 253 μmol) and 25% aq. NH₃ (75 μL, 1 mmol) in DMF (5 mL) was stirred at rt for 1 hour. The reaction mixture was diluted with DCM-MeOH (10:1) and washed with brine (until pH became neutral, MeOH was added to the organic phase whenever precipitates were formed), dried (Na₂SO₄) and concentrated in vacuo. The red residue was purified by FC (SiO₂, RediSep R_{f} 24 g cartridge, dry-load, DCM—DCM/MeOH/H₂O 90:10:1) to give a red solid (67 mg, 83%). HPLC: t_{R}=7.2 min (20-100); λₘₐₓ=435, 415, 357, 311, 265, 250 nm. ESI-MS: m/z 318.1 [M+H]⁺. ¹H NMR (400 MHz, MeOD-d₃ with TFA-d): δ=9.64 (d, J=2.3 Hz, 1H), 8.82 (d, J=2.0 Hz, 1H), 8.72 (dd, J=9.4, 2.3 Hz, 1H), 8.17 (dd, J=9.0, 2.0 Hz, 1H), 7.96 (d, J=9.4 Hz, 1H), 7.81 (d, J=9.0 Hz, 1H) ppm. ³C NMR (101 MHz, MeOD-d₃ with TFA-d): δ=159.4, 143.5, 142.2, 139.6, 138.8, 129.0, 126.8, 122.1, 120.8, 120.2, 118.2, 113.7, 111.0 ppm.

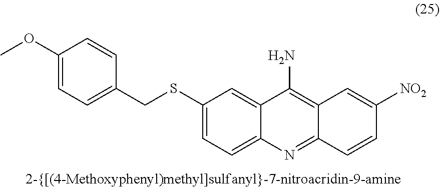 [SMILES: COc1ccc(CSc2ccc3nc4ccc([N+](=O)[O-])cc4c(N)c3c2)cc1]

Compound 24 (24 mg, 75 μmol), Pd₂(dba)₃ (4 mg, 4 μmol) and Xantphos (5 mg, 8 μmol) were placed in a heat dried Schlenk tube filled with Ar, which was evacuated-backfilled with Ar (3×). DMF (1 mL), DIPEA (26 μL, 150 μmol) and p-methoxybenzylmercaptane (15 mg, 90 μmol) were added and the reaction mixture was immediately heated up to 100° C. After 2 h of stirring at the same temperature, the reaction mixture was allowed to cool down to rt, diluted with DCM-MeOH (10:1), washed with 10% aq. Na₂SO₃— aq. sat. NaHCO₃ (1:1) and brine, dried (Na₂SO₄) and concentrated in vacuo. The residue was purified by FC (SiO₂, RediSep R_{f} 24 g cartridge, dry-load, DCM—DCM/MeOH/H₂O 90:10:1) to give a red solid (23 mg, 79%). HPLC: t_{R}=9.5 min (20-100); λₘₐₓ=434, 341, 273 nm. ESI-MS: m/z 392.3 [M+H]⁺. ¹H NMR (400 MHz, MeOD-d₃ with TFA-d): δ=9.66 (d, J=2.3 Hz, 1H), 8.71 (dd, J=9.4, 2.3 Hz, 1H), 8.46 (d, J=2.0 Hz, 1H), 8.01 (dd, J=9.0, 2.0 Hz, 1H), 7.93 (d, J=9.4 Hz, 1H), 7.78 (d, J=9.0 Hz, 1H), 7.24-7.30 (m, 2H), 6.79-6.85 (m, 2H), 4.32 (s, 2H), 3.74 (s, 3H) ppm.

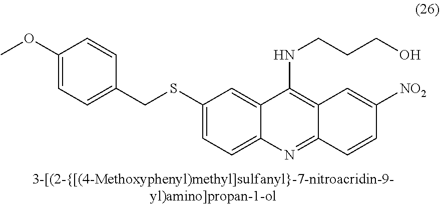 [SMILES: COc1ccc(CSc2ccc3nc4ccc([N+](=O)[O-])cc4c(NCCCO)c3c2)cc1]

Compound 21 (290 mg, 0.77 mmol), Pd₂(dba)₃ (37 mg, 0.04 mmol) and Xantphos (46 mg, 0.08 mmol) were placed in a heat dried Schlenk tube filled with Ar, which was evacuated-backfilled with Ar (3×). DMF (7.7 mL), DIPEA (268 μL, 1.54 mmol) and PMBSH (142 μg, 0.92 mmol) were added and the reaction mixture was immediately heated up to 100° C. After 2 h of stirring at the same temperature, the reaction mixture was allowed to cool down to rt, diluted with DCM-MeOH (10:1), washed with 10% aq. Na₂SO₃— aq. sat. NaHCO₃ (1:1) and brine, dried (Na₂SO₄) and concentrated in vacuo. The residue was purified by FC (SiO₂, RediSep R_{f} 24 g cartridge, dry-load, DCM—DCM/MeOH/H₂O 90:10:1) to give a red solid (325 mg, 94%). TLC (SiO₂): R_{f}=0.35 (DCM/MeOH/H₂O 90:10:1). HPLC: t_{R}=9.6 min (20-100); λₘₐₓ=441, 348, 274 nm. ESI-MS: m/z 450.3 [M+H]⁺. HRMS: m/z 450.1491 ([M+H]⁺) calculated for C₂₄H₂₄N₃O₄S⁺: 450.1482 (Δ2.0 ppm). ¹H NMR (400 MHz, MeOD-d₃ with TFA-d): δ=9.43 (d, J=2.0 Hz, 1H), 8.67 (dd, J=9.4, 2.3 Hz, 1H), 8.24 (br s, 1H), 7.92-8.05 (m, 1H), 7.88 (d, J=9.4 Hz, 1H), 7.75 (d, J=9.0 Hz, 1H), 7.26 (d, J=8.6 Hz, 2H), 6.76-6.88 (m, 2H), 4.28 (s, 2H), 4.00-4.63 (m, 2H), 3.82-3.99 (m, 2H), 3.74 (s, 3H), 1.93-2.31 (m, 2H) ppm.

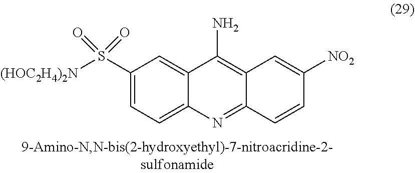

Compound 29 was synthesized from a corresponding sulfide 25 through a sulfonyl chloride intermediate 27 generated as described in Y-M. Pu, A. Christesen, Y.-Y. Ku, Tetrahedron Lett. 2010, 51, 418-421. Compound 25 (10 mg, 25 μmol) was suspended in the mixture of MeCN (500 μL), AcOH (20 μL) and H₂O (12 μL) at 0° C., further 100 μL of DCM was added. DCDMH (10 mg, 50 μmol) was added in portions. The color changed to yellow. The formation of the sulfonyl chloride intermediate was monitored by HPLC (t_{R}=7.3 min; λₘₐₓ=416 sh., 401, 357, 300, 261 nm; method 20-100). After 40 min of stirring at 0° C., the educt was fully converted. Mixture of DEA (10 mg, 100 μmol), DIPEA (100 μL) and ACN (100 μL) was added, and the reaction mixture was allowed to warm to rt. The reaction mixture became a clear orange solution. After 15 min of stirring at rt, the reaction mixture was concentrated in vacuo. The residue was dissolved in 10 mL of 0.1% aq. TFA and purified by FC (C18, 30C18AQ-F0012 cartridge, ACN—0.1% aq. TFA). Good fractions were pooled, basified with sat. NaHCO₃, and the product was extracted into EtOAc-MeOH (10:1). The combined organic phase was washed with brine, dried (Na₂SO₄) and concentrated in vacuo to give a light orange solid (1 mg, 10%). TLC (SiO₂): R_{f}=0.2 (DCM/MeOH/H₂O 90:10:1). HPLC: t_{R}=10.3 min (5-50); λₘₐₓ=424, 404, 356, 305, 260 nm. ESI-MS: m/z 407.3 [M+H]⁺.

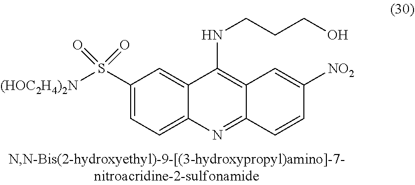

Compound 30 was synthesized from a corresponding sulfide 26 through a sulfonyl chloride intermediate 28 generated as described in Y-M. Pu, A. Christesen, Y.-Y. Ku, Tetrahedron Lett. 2010, 51, 418-421. Compound 26 (61 mg, 136 μmol) was suspended in 3 mL of MeCN/AcOH/H₂O (20:0.75:0.5) at 0° C. DCDMH (75 mg, 381 μmol) was added portionwise. The color of suspension changed to yellow. The reaction mixture was stirred at 0° C. for 30 min, then 30 min at rt. The formation of the sulfonyl chloride intermediate was assessed by HPLC (t_{R}=7.7 min, λₘₐₓ=423, 406, 360, 302, 265 nm, method 20-100), the educt was fully converted. The solvent was removed in vacuo (35° C., <20 mbar). DEA (75 mg) in ACN (15 mL) was added. The color changed immediately to orange-brown. After 1 h of stirring at rt, the reaction mixture was diluted with EtOAc-MeOH (10:1), washed with sat. NaHCO₃ and brine, dried (Na₂SO₄) and concentrated in vacuo. The residue was purified by FC (SiO₂, RediSep Rf 24 g cartridge, dry-load, DCM—DCM/MeOH/H₂O 90:10:1) to give a light orange solid (52 mg, 82%). TLC (SiO₂): R_{f}=0.2 (DCM/MeOH/H₂O 90:10:1). HPLC: t_{R}=10.5 min (5-50); λₘₐₓ=428, 410, 360, 307, 267 nm. HRMS: m/z 465.1441 ([M+H]⁺) calculated for C₂₀H₂₅N40₇S⁺: 465.1438 (Δ0.6 ppm). ¹H NMR (400 MHz, MeOD-d₃ with TFA-d): δ=9.33-9.63 (m, 1H), 8.95 (s, 1H), 8.73 (br dd, J=9.0, 0.8 Hz, 1H), 8.35 (br d, J=9.0 Hz, 1H), 7.95 (d, J=9.0 Hz, 2H), 4.31-4.55 (m, 2H), 3.83-4.04 (m, 2H), 3.74 (t, J=5.9, 5.5 Hz, 4H), 3.42 (t, J=5.5 Hz, 4H), 2.25 (quin, J=6.7, 6.3, 5.9, 5.5 Hz, 2H) ppm.

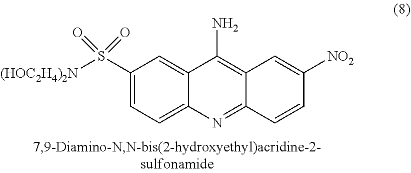

Compound 29 (1 mg, 2.5 μmol) was dissolved in MeOH (1 mL), containing AcOH (10 μL), under Ar. A 10% Pd/C catalyst (2 mg) was added, and hydrogen atmosphere was applied. After 1 hour of stirring at rt, orange fluorescent solution was filtered through a pad of Celite and concentrated in vacuo. The residue was dissolved in 10 mL of H₂O (with 0.1% TFA) and purified by FC (C18, 15C18HP-F0012 cartridge, ACN—0.1% aq. TFA). Good fractions were pooled and lyophilized to give a red solid (0.8 mg, TFA salt). HPLC: t_{R}=9.0 min (5-50); λₘₐₓ=453 sh., 422, 401, 302 sh., 270 nm. ESI-MS: m/z 377.3 [M+H]⁺.

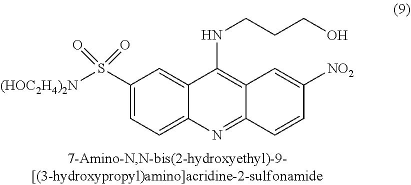

Compound 30 (5 mg, 11 μmol) was dissolved in MeOH (10 mL), containing AcOH (10 μL), under Ar. A 10% Pd/C catalyst (1 mg) was added, and hydrogen atmosphere was applied. After 1 hour of stirring at rt, orange fluorescent solution was filtered through a pad of Celite and concentrated in vacuo. The residue was dissolved in 10 mL of H₂O (with 0.1% TFA) and purified by FC (C18, 15C18HP-F0012 cartridge, ACN—0.1% aq. TFA). Good fractions were pooled and lyophilized (2×) to give a red solid (4.94 mg, 84%, TFA salt). HPLC: t_{R}=9.0 min (5-50); λₘₐₓ=430, 410, 272 nm. HRMS: m/z 435.1693 ([M+H]⁺) calculated for C₂₀H₂₇N₄O₅S⁺: 435.1697 (Δ0.9 ppm). ¹H NMR (400 MHz, D₂O): δ=8.35 (d, J=2.0 Hz, 1H), 7.95 (dd, J=9.0, 2.0 Hz, 1H), 7.48 (d, J=9.0 Hz, 1H), 7.25-7.38 (m, 2H), 7.07 (br s, 1H), 3.97 (t, J=7.0, 6.7 Hz, 2H), 3.81 (t, J=5.9, 5.5 Hz, 2H), 3.70 (t, J=5.9, 5.5 Hz, 4H), 3.37 (t, J=5.9, 5.5 Hz, 4H), 2.04 (quin, J=6.7, 6.3, 5.9 Hz, 2H) ppm. ¹⁹F NMR (376 MHz, D₂O): δ=−75.53 ppm.

Reaction of sulfide 26 with 3-hydroxyazetidine through a sulfonyl chloride intermediate.

Compound 26 (22 mg, 50 μmol) was suspended in 1 mL of MeCN/AcOH/H₂O (20:0.75:0.5) at 0° C. DCDMH (25 mg, 125 μmol) was added portionwise. The color of suspension changed to yellow. The reaction mixture was stirred at 0° C. for 30 min, then 30 min at rt. The formation of the sulfonyl chloride intermediate 26-SO₂Cl was assessed by HPLC (t_{R}=7.5 min, λₘₐₓ=423, 406, 360, 302, 265 nm, method 20-100), the educt was fully converted. The solvent was removed in vacuo (35° C., <20 mbar). MeCN (10 mL), 3-hydroxyazetidine hydrochloride (50 mg, 456 μmol) and DIPEA (15 mL) were added. The color changed immediately to orange-brown. After 1 h of stirring at rt, the reaction mixture was stored at −20° C. overnight. The reaction mixture was diluted with EtOAc-MeOH (10:1), washed with sat. NaHCO₃ and brine, dried (Na₂SO₄) and concentrated in vacuo. The residue was purified by FC (SiO₂, SNAP Ultra 10 g cartridge, dry-load, DCM—DCM/MeOH/H₂O 90:10:1) to give the desired product 31 (3 mg, 14%) as a brown solid and side-product 32 (6 mg, 28%) as a light orange solid.

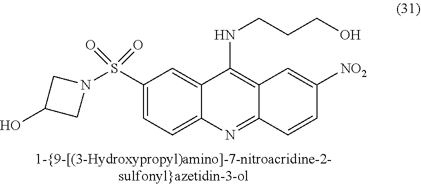 [SMILES: O=[N+]([O-])c1ccc2nc3ccc(S(=O)(=O)N4CC(O)C4)cc3c(NCCCO)c2c1]

HPLC: t_{R}=11.0 min (5-50); λₘₐₓ=428, 410, 359, 305, 267 nm. ESI-MS: m/z 433.2 [M+H]⁺. HRMS: m/z 433.1177 ([M+H]⁺) calculated for C₁₉H₂₁N₄O₆S⁺: 433.1176 (Δ0.2 ppm). ¹H NMR (400 MHz, MeOD-d₃ with TFA-d): δ=9.48 (br s, 1H), 8.94 (d, J=1.6 Hz, 1H), 8.76 (dd, J=9.4, 2.0 Hz, 1H), 8.35 (dd, J=9.0, 1.6 Hz, 1H), 8.02 (d, J=9.0 Hz, 1H), 7.96 (d, J=9.4 Hz, 1H), 4.43 (quin, J=6.3, 5.9, 5.5 Hz, 1H), 4.31-4.55 (m, 2H), 4.02-4.15 (m, 2H), 3.82-4.01 (m, 2H), 3.59 (dd, J=8.6, 5.5 Hz, 2H), 2.26 (quin, J=6.3, 5.9, 5.5 Hz, 2H) ppm.

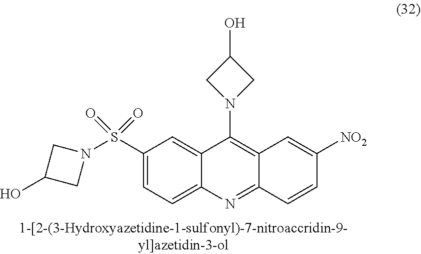 [SMILES: O=[N+]([O-])c1ccc2nc3ccc(S(=O)(=O)N4CC(O)C4)cc3c(N3CC(O)C3)c2c1]

HPLC: t_{R}=10.5 min (5-50); λₘₐₓ=425, 410, 361, 305, 269 nm. ESI-MS: m/z 431.3 [M+H]⁺. HRMS: m/z 431.1022 ([M+H]⁺) calculated for C₁₉H₁₉N₄O₆S⁻: 431.1020 (Δ0.5 ppm). ¹H NMR (400 MHz, MeOD-d₃ with TFA-d): δ=9.00 (d, J=2.3 Hz, 1H), 8.68 (dd, J=9.4, 2.3 Hz, 1H), 8.51 (d, J=2.0 Hz, 1H), 8.25 (dd, J=9.0, 2.0 Hz, 1H), 7.95 (d, J=9.0 Hz, 1H), 7.91 (d, J=9.4 Hz, 1H), 5.27-5.38 (m, 2H), 5.15-5.26 (m, 2H), 4.88 (dddd, J=6.6, 4.4 Hz, 1H), 4.45 (dddd, J=6.6, 5.5 Hz, 1H), 4.02-4.12 (m, 2H), 3.54-3.63 (m, 2H) ppm.

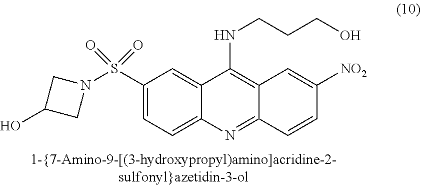 [SMILES: O=[N+]([O-])c1ccc2nc3ccc(S(=O)(=O)N4CC(O)C4)cc3c(NCCCO)c2c1]

Compound 31 (3 mg, 7 μmol) was dissolved in MeOH (10 mL), containing AcOH (10 μL), under Ar. A 10% Pd/C catalyst (1 mg) was added, and hydrogen atmosphere was applied. After 1 hour of stirring at rt, orange fluorescent solution was filtered through a pad of Celite and concentrated in vacuo. The residue was dissolved in 10 mL of H₂O (with 0.1% TFA) and purified by FC (C18, 15C18HP-F0012 cartridge, ACN—0.1% aq. TFA). Good fractions were pooled and lyophilized to give a red powder (2.26 mg, 63%, TFA salt). HPLC: t_{R}=9.7 min (5-50); λₘₐₓ=431, 410, 303, 273 nm. HRMS: m/z 403.1439 ([M+H]⁺) calculated for C₁₉H₂₃N₄O₄S⁺: 403.1435 (Δ1.0 ppm). ¹H NMR (400 MHz, D₂O): δ=8.45 (d, J=1.6 Hz, 1H), 8.00 (dd, J=9.0, 1.6 Hz, 1H), 7.64 (d, J=9.4 Hz, 1H), 7.37-7.48 (m, 2H), 7.25 (br s, 1H), 4.45 (quin, J=6.7, 6.3, 5.5, 5.1 Hz, 1H), 4.01-4.13 (m, 4H), 3.82 (t, J=5.9, 5.5 Hz, 2H), 3.60 (d, J=5.1 Hz, 1H), 3.57 (d, J=5.1 Hz, 1H), 2.07 (quin, J=6.7, 6.3, 5.9 Hz, 2H) ppm.

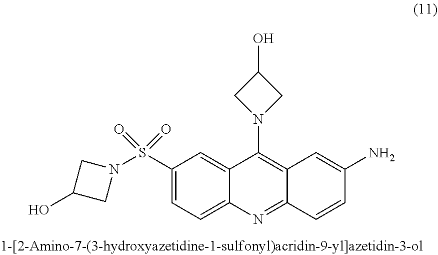 [SMILES: Nc1ccc2nc3ccc(S(=O)(=O)N4CC(O)C4)cc3c(N3CC(O)C3)c2c1]

Compound 32 (6 mg, 14 μmol) was dissolved in MeOH (10 mL), containing AcOH (10 μL), under Ar. A 10% Pd/C catalyst (1 mg) was added, and hydrogen atmosphere was applied. After 1 hour of stirring at rt, orange fluorescent solution was filtered through a pad of Celite and concentrated in vacuo. The residue was dissolved in 10 mL of H₂O (with 0.1% TFA) and purified by FC (C18, 15C18HP-F0012 cartridge, ACN—0.1% aq. TFA). Good fractions were pooled and lyophilized to give a red powder (6 mg, 86%, TFA salt). HPLC: t_{R}=8.6 min (5-50); λₘₐₓ=430, 410, 277 nm. HRMS: m/z 401.1281 ([M+H]⁺) calculated for Cl₉H₂₁N₄O₄S⁺: 401.1278 (Δ0.7 ppm). 1H NMR (400 MHz, MeOD-d₃): δ=8.47 (d, J=1.6 Hz, 1H), 8.08 (dd, J=9.0, 1.6 Hz, 1H), 7.85 (d, J=9.0 Hz, 1H), 7.64 (d, J=9.0 Hz, 1H), 7.47 (dd, J=9.0, 2.0 Hz, 1H), 7.32 (d, J=2.0 Hz, 1H), 5.17-5.43 (m, 2H), 4.98-5.16 (m, 2H), 4.83-4.91 (m, 1H), 4.45 (dq, J=6.3, 5.9 Hz, 1H), 4.06 (t, J=7.8 Hz, 2H), 3.57 (dd, J=8.6, 5.5 Hz, 2H) ppm. ¹³C NMR (101 MHz, MeOD-d₃, HSQC): δ=158.4, 146.0, 142.4, 134.5, 132.4 (CH), 129.7 (CH), 127.9 (CH), 127.8, 120.5 (2×CH), 115.5, 111.3, 107.3 (CH), 72.6 (2×CH₂), 63.4 (CH), 61.6 (2×CH₂), 60.8 (CH) ppm. ¹⁹F NMR (376 MHz, MeOD-d₃): δ=−76.71 ppm.

### Example 2

### Synthesis of Aminoacridine Dye-Glucose Conjugates

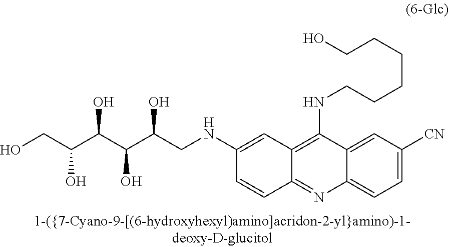 [SMILES: N#Cc1ccc2nc3ccc(NC[C@H](O)[C@@H](O)[C@H](O)[C@H](O)CO)cc3c(NCCCCCCO)c2c1]

Compound 6 (20 mg, 36 μmol), glucose (32 mg, 178 μmol) and AcOH (4 mg, 71 μmol) were mixed in 1 mL of water. NaBH₃CN (22 mg, 356 μmol) in 0.5 mL of MeOH was added. The reaction mixture was heated to 60° C. The reaction progress was monitored by HPLC. After 5 h of stirring at 60° C., the reaction mixture was cooled to rt, diluted with 10 mL of 0.1% aq. TFA and purified by FC (C18, 30C18AQ-F0025 cartridge, ACN—0.1% aq. TFA). The good fractions were pooled and lyophilized to give the title product 6-Glc (10 mg, TFA salt) as a red-purple solid. HPLC: t_{R}=10.9 min (5-50). ESI-MS: m/z 499.4 [M+H]⁺. HRMS: m/z 499.2553 ([M+H]⁺) calculated for C₂₆H₃₄N₄O₆⁺: 499.2551 (Δ0.4 ppm). ¹H NMR (400 MHz, D₂O): 6=8.12 (s, 1H), 7.76 (dd, J=9.0, 1.6 Hz, 1H), 7.39 (d, J=9.0 Hz, 1H), 7.30 (dd, J=9.0, 2.0 Hz, 1H), 7.25 (d, J=9.0 Hz, 1H), 6.62 (d, J=2.0 Hz, 1H), 4.06 (ddtd, J=5.1, 4.3, 3.9, 0.9 Hz, 1H), 3.91 (dd, J=5.3, 2.2 Hz, 1H), 3.86 (dd, J=15.7, 2.7 Hz, 1H), 3.80-3.89 (m, 1H), 3.66-3.78 (m, 4H), 3.57 (t, J=6.7 Hz, 2H), 3.33 (dd, J=13.3, 3.9 Hz, 1H), 3.18 (dd, J=13.3, 8.2 Hz, 1H), 1.76 (quin, J=7.0 Hz, 2H), 1.54 (quin, J=6.7 Hz, 2H), 1.30-1.45 (m, 4H) ppm. 19F NMR (376 MHz, D₂O): δ=−75.50 ppm.

General method for labelling of glucose with model compounds (7, 9-11). 1.5 mL Brand® micro tube with screw cap was charged with dye (1 equiv., 0.1 M solution in water), glucose (1 equiv., 0.1 M solution in water), malonic acid (10 equiv., 1 M solution in DMSO) and 2-picoline-borane complex (10 equiv., 1 M solution in DMSO). After vortexing for 10 s, the reaction mixture was incubated in an Eppendorf ThermoMixer® with shaking (400-600 rpm) at 40° C. for 18 h. The reaction mixture was cooled to rt, diluted with 10 mL of 0.1% aq. TFA and purified by FC (C18, 15C18HP-F0012 cartridge, ACN—0.1% aq. TFA). The good fractions were pooled and lyophilized.

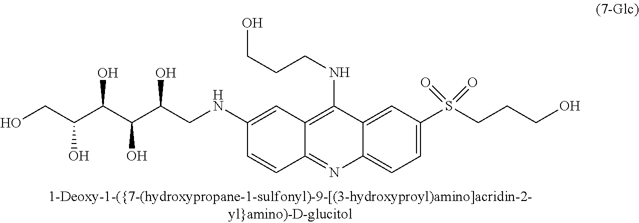 [SMILES: O=S(=O)(CCCO)c1ccc2nc3ccc(NC[C@H](O)[C@@H](O)[C@H](O)[C@H](O)CO)cc3c(NCCCO)c2c1]

The title compound 7-Glc (0.8 μmol, TFA salt) was obtained as an orange-red solid from dye 7 (1.3 μmol) according to the general method for labelling of glucose. HPLC: t_{R}=9.1 min (5-50); λₘₐₓ=482, 319, 275 nm. ESI-MS: m/z 554.3 [M+H]⁺. HRMS: m/z 554.2160 ([M+H]⁺) calculated for C₂₅H₃₆N₃O₉S⁺: 554.2167 (Δ1.3 ppm). 1H NMR (400 MHz, D₂O): δ=8.63 (d, J=1.0 Hz, 1H), 8.06 (d, J=9.0 Hz, 1H), 7.69 (dd, J=9.0, 0.4 Hz, 1H), 7.46 (d, J=9.0 Hz, 1H), 7.38-7.44 (m, 1H), 6.91 (s, 1H), 4.03-4.20 (m, 3H), 3.91 (dd, J=5.5, 2.2 Hz, 1H), 3.78-3.88 (m, 4H), 3.75 (dd, J=8.2, 2.2 Hz, 1H), 3.64-3.71 (m, 1H), 3.62 (t, J=6.3 Hz, 2H), 3.46-3.53 (m, 2H), 3.43 (dd, J=13.3, 3.5 Hz, 1H), 3.26 (dd, J=13.3, 8.2 Hz, 1H), 2.04-2.17 (m, 2H), 1.85-1.97 (m, 2H) ppm. ¹⁹F NMR (376 MHz, D₂O): δ=−75.53 ppm.

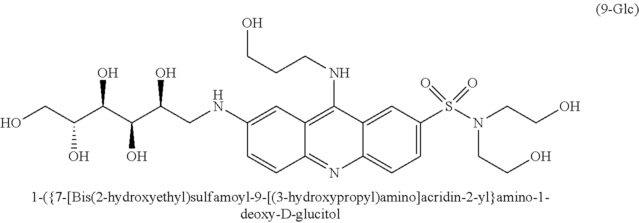 [SMILES: O=S(=O)(c1ccc2nc3ccc(NC[C@H](O)[C@@H](O)[C@H](O)[C@H](O)CO)cc3c(NCCCO)c2c1)N(CCO)CCO]

The title compound 9-Glc (0.7 μmol, TFA salt) was obtained as an orange-red solid from dye 9 (1.6 μmol) according to the general method for labelling of glucose. HPLC: t_{R}=9.1 min (5-50); λₘₐₓ=480, 319, 276 nm. ESI-MS: m/z 599.3 [M+H]⁺. HRMS: m/z 599.2381 ([M+H]⁺) calculated for C₂₆H₃₉N₄O₁₀S⁺: 599.2381 (Δ0.0 ppm). ¹H NMR (400 MHz, D₂O): δ=8.53 (s, 1H), 8.01 (dd, J=9.0, 1.0 Hz, 1H), 7.61 (d, J=9.0 Hz, 1H), 7.36-7.47 (m, 2H), 6.88 (s, 1H), 4.01-4.17 (m, 3H), 3.93 (dd, J=5.5, 2.0 Hz, 1H), 3.80-3.89 (m, 4H), 3.75-3.80 (m, 1H), 3.62-3.75 (m, 5H), 3.33-3.48 (m, 5H), 3.27 (dd, J=13.1, 8.4 Hz, 1H), 2.04-2.17 (m, 2H) ppm. ¹⁹F NMR (376 MHz, D₂O): δ=−75.52 ppm.

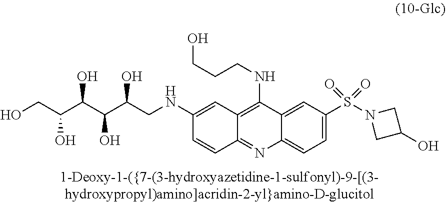 [SMILES: O=S(=O)(c1ccc2nc3ccc(NC[C@H](O)[C@@H](O)[C@H](O)[C@H](O)CO)cc3c(NCCCO)c2c1)N1CC(O)C1]

The title compound 10-Glc (0.6 μmol, TFA salt) was obtained as an orange-red solid from dye 10 (1.5 μmol) according to the general method for labelling of glucose. HPLC: t_{R}=9.6 min (5-50); λₘₐₓ=482, 320, 276 nm. ESI-MS: m/z 567.3 [M+H]⁺. HRMS: m/z 567.2120 ([M+H]⁺) calculated for C₂₅H₃₅N₄O₉S⁺: 567.2119 (Δ0.2 ppm). 1H NMR (400 MHz, D₂O): δ=8.53 (s, 1H), 7.95-8.12 (m, 1H), 7.60-7.82 (m, 1H), 7.33-7.57 (m, 2H), 6.82-7.00 (m, 1H), 4.39-4.56 (m, 1H), 4.00-4.25 (m, 5H), 3.90-3.97 (m, 1H), 3.80-3.89 (m, 4H), 3.74-3.80 (m, 1H), 3.66-3.74 (m, 1H), 3.56-3.65 (m, 2H), 3.37-3.51 (m, 1H), 3.20-3.32 (m, 1H), 2.07-2.18 (m, 2H) ppm. ¹⁹F NMR (376 MHz, D₂O): δ=−75.52 ppm.

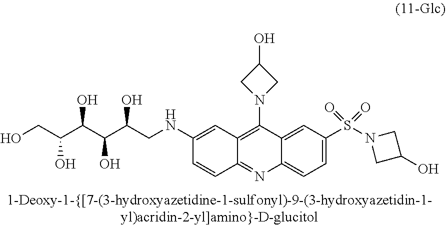 [SMILES: O=S(=O)(c1ccc2nc3ccc(NC[C@H](O)[C@@H](O)[C@H](O)[C@H](O)CO)cc3c(N3CC(O)C3)c2c1)N1CC(O)C1]

The title compound 11-Glc (1.3 μmol, AcOH salt) was obtained as a red solid from dye 11 (5.1 μmol) according to the general method for labelling of glucose. The material was further purified by FC (C18, 15C18HP-F0012 cartridge, ACN—0.1% aq. AcOH). The product appeared to be unstable in the presence of TFA (by HPLC). HPLC: t_{R}=8.8 min (5-50); λₘₐₓ=485, 431, 320, 277 nm. HRMS: m/z 565.1959 ([M+H]⁺) calculated for C₂₅H₃₃N₄O₉S⁺: 565.1963 (Δ0.7 ppm). ¹H NMR (400 MHz, D₂O): δ=8.12 (d, J=1.2 Hz, 1H), 7.96 (dd, J=9.0, 1.2 Hz, 1H), 7.54 (br d, J=9.0 Hz, 1H), 7.37-7.48 (m, 2H), 6.87 (s, 1H), 5.04-5.25 (m, 2H), 4.87-5.01 (m, 2H), 4.78-4.82 (m, 1H), 4.47 (dt, J=11.7, 5.9 Hz, 1H), 4.00-4.16 (m, 3H), 3.91 (dd, J=5.5, 2.3 Hz, 1H), 3.78-3.86 (m, 2H), 3.75 (dd, J=8.0, 2.2 Hz, 1H), 3.64-3.71 (m, 1H), 3.61 (dd, J=9.2, 5.3 Hz, 2H), 3.47 (dd, J=13.5, 4.1 Hz, 1H), 3.29 (dd, J=13.5, 8.0 Hz, 1H), 1.88 (s, 2H, AcOH) ppm.

### Example 3

### Synthesis of Phosphorylated Acridines (and Acridone)

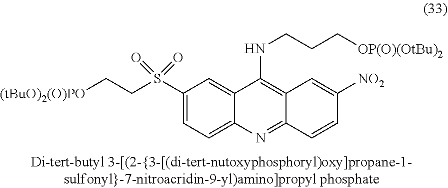

Compound 22 (330 mg, 0.79 mmol) and 1H-terazole (330 mg, 4.72 mmol) were placed in Ar-filled flask, which was evacuated-backfilled with Ar (3×). DMF (8 mL) and (t-BuO)₂PNi-Pr₂ (994 μL, 3.15 mmol) were added, and the reaction mixture was stirred 1 h at rt. Formation of the phosphite intermediate was monitored by HPLC (t_{R}=14.3 min, 5-50). Then 50% aq. H₂O₂ (358 μL, 7.3 mmol) was added in one portion. After 30 min of stirring at rt, the reaction mixture was diluted with DCM (200 mL), washed with 10% aq. Na₂SO₃— aq. sat. NaHCO₃ (100 mL, 1:1) and brine, dried (Na₂SO₄) and concentrated in vacuo. The residue was purified by FC (SiO₂, 15SIHP-F0040 cartridge, DCM/MeOH with 2-5% MeOH gradient) to give a light orange solid (260 mg, 41%). TLC (SiO₂): R_{f}=0.3 (DCM/MeOH 20:1). HPLC: t_{R}=10.9 min (20-100); t_{R}=17.6 min (5-50); λₘₐₓ=427, 410, 357, 303, 267 nm. ESI-MS: m/z 804.5 [M+H]⁺.

HRMS: m/z 804.3056 ([M+H]⁺) calculated for C₃₅H₅₆N₃O₁₂P₂S⁺: 804.3054 (Δ0.2 ppm). ¹H NMR (400 MHz, MeOD-d₃ with TFA-d): δ=9.29-9.75 (m, 1H), 8.89-9.28 (m, 1H), 8.75 (dd, J=9.4, 2.3 Hz, 1H), 8.42 (dd, J=9.0, 1.6 Hz, 1H), 8.00 (d, J=9.0 Hz, 1H), 7.95 (d, J=9.4 Hz, 1H), 4.36 (t, J=6.7 Hz, 2H), 4.19 (q, J=6.7, 6.3, 5.9 Hz, 2H), 4.06 (q, J=7.4, 6.3 Hz, 2H), 3.42-3.52 (m, 2H), 2.44 (quin, J=6.3 Hz, 2H), 2.04-2.17 (m, 2H), 1.44 (s, 9H), 1.44 (s, 9H), 1.42 (s, 18H) ppm. ³¹P NMR (162 MHz, CDCl₃): δ=−7.99 (br s, 1P), −10.13 (s, 1P) ppm.

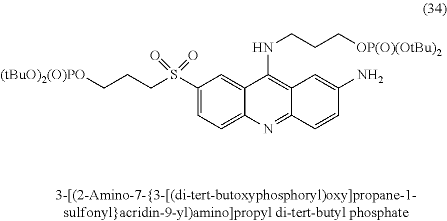

Compound 33 (50 mg, 62 μmol) was dissolved in MeOH (4 mL), containing AcOH (20 μL), under Ar. A 10% Pd/C catalyst (5 mg) was added, and hydrogen atmosphere was applied. After 1 h of stirring at rt, orange fluorescent solution was filtered through a pad of Celite and concentrated in vacuo. The residue was dissolved in 10 mL of ACN/0.1% aq. TFA (1:5) and purified by FC (C18, 30C18AQ-F0025 cartridge, ACN—0.1% aq. TFA). Good fractions were pooled and lyophilized to give a red powder (42 mg, 76%, TFA salt). HPLC: t_{R}=10.3 min (20-100). ESI-MS: m/z 774.6 [M+H]⁺. HRMS: m/z 774.3315 ([M+H]⁺) calculated for C₃₅H₅₈N₃O₁₀P₂S⁺: 774.3313 (Δ0.3 ppm). ¹H NMR (400 MHz, MeOD-d₃): δ=9.02 (d, J=1.6 Hz, 1H), 8.21 (dd, J=9.0, 1.6 Hz, 1H), 7.91 (d, J=9.0 Hz, 1H), 7.69 (dd, J=7.4, 2.3 Hz, 1H), 7.53 (s, 1H), 7.54 (dd, J=7.4, 2.3 Hz, 1H), 4.32 (t, J=7.0, 6.7 Hz, 2H), 4.20 (q, J=6.3, 5.9 Hz, 2H), 4.06 (q, J=7.8, 6.3, 5.9 Hz, 2H), 3.45 (m, J=7.8, 7.4, 2.3, 2.0 Hz, 2H), 2.38 (quin, J=6.3, 5.9 Hz, 2H), 2.05-2.16 (m, 2H), 1.45 (s, 18H), 1.44 (s, 18H) ppm. ³¹P NMR (162 MHz, MeOD-D₃): δ=−10.61 (s, 1P), −10.62 (s, 1P) ppm.

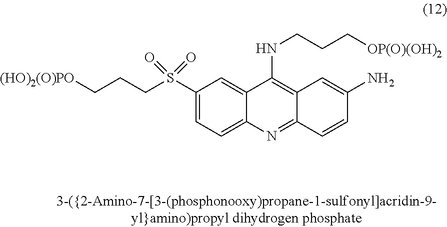

Compound 34 (43 mg, 48 μmol) was stirred in 5% TFA in DCM (4 mL) for 4 h at rt under Ar. The reaction mixture was then concentrated in vacuo (without heating bath). The residue was dissolved in 10 mL of TEAAc buffer (1.0 M, pH 7) and purified by FC (C18, 30C18AQ-F0025, ACN—10 mM TEAAc pH 7). Good fractions were pooled and lyophilized to give a red powder (20 mg, 65%, TEA salt). HPLC (Knauer Eurospher II 100-5 C18A): t_{R}=9.3 min (TEAB-O-25); λₘₐₓ=455, 303, 273 nm. ESI-MS: m/z 548.2 [M−H]⁻. HRMS: m/z 550.0805 ([M+H]⁺) calculated for C₁₉H₂₆N₃O₁₀P₂S⁺: 550.0809 (Δ0.7 ppm). 1H NMR (400 MHz, D₂O): δ=8.35 (s, 1H), 7.94 (dd, J=9.0, 1.6 Hz, 1H), 7.49 (d, J=9.0 Hz, 1H), 6.94-7.13 (m, 2H), 6.83 (s, 1H), 4.06-4.22 (m, 2H), 3.85-3.98 (m, 4H), 3.46-3.59 (m, 2H), 3.18 (q, J=7.2 Hz, 6H, TEA), 2.09-2.20 (m, 2H), 1.98-2.08 (m, 2H), 1.26 (t, J=7.2 Hz, 9H, TEA) ppm. ³¹P NMR (162 MHz, D₂O): δ=0.35 (s, 1P), 0.25 (s, 1P) ppm.

Reaction of 30 with di-tert-butyl N,N-diisopropylphosphoramidite with H₂O₂ as oxidant.

Compound 30 (57 mg, 0.12 mmol) and 1H-terazole (78 mg, 1.11 mmol) were placed in Ar-filled flask, which was evacuated-backfilled with Ar (3×). DMF (1.25 mL) and (t-BuO)₂PNi-Pr₂ (233 μL, 0.74 mmol) were added, and the reaction mixture was stirred 1 h at rt. Formation of the phosphite intermediate was monitored by HPLC (t_{R}=9.2 min, 20-100). Then 50% aq. H₂O₂ (278 μL, 4.9 mmol) was added in one portion. After 30 min of stirring at rt, the reaction mixture was diluted with DCM (200 mL), washed with 10% aq. Na₂SO₃— aq. sat. NaHCO₃ (100 mL, 1:1) and brine, dried (Na₂SO₄) and concentrated in vacuo. The residue was purified by FC (SiO₂, 30SIHP-F0025 cartridge, DCM/MeOH with 1-5% MeOH gradient) to give the desired product 35 (58 mg, 47%) as an orange oil and a side-product 36 (12 mg, 13%) as a yellow powder.

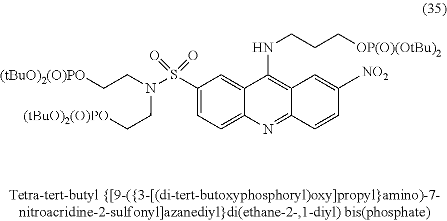

HPLC: t_{R}=12.3 min (20-100); λₘₐₓ=429, 412, 358, 307, 268 nm. HRMS: m/z 1041.4168 ([M+H]⁺) calculated for C₄₄H₇₆N₄O₁₆P₃S⁺: 1041.4184 (Δ1.5 ppm). ¹H NMR (400 MHz, MeOD-d₃ with TFA-d): δ=9.29-9.80 (m, 1H), 8.83-9.23 (m, 1H), 8.76 (dd, J=9.4, 2.3 Hz, 1H), 8.40 (dd, J=9.0, 2.0 Hz, 1H), 7.97 (d, J=9.0 Hz, 1H), 7.96 (d, J=9.4 Hz, 1H), 4.37 (t, J=7.0, 6.7 Hz, 2H), 4.21 (q, J=6.7, 5.9 Hz, 2H), 4.13 (q, J=6.3, 5.9, 5.5 Hz, 4H), 3.66 (t, J=5.5 Hz, 4H), 2.47 (quin, J=6.3, 5.9 Hz, 2H), 1.46 (s, 36H), 1.45 (s, 18H) ppm. ³¹P NMR (162 MHz, MeOD-d₃ with TFA-d): δ=−10.23 (s, 1P), −10.90 (s, 2P) ppm.

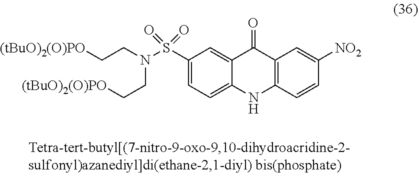

HPLC: t_{R}=12.6 min (20-100); λₘₐₓ=354, 298, 249 nm. ESI-MS: m/z 790.4 [M−H]⁻. HRMS: m/z 814.2515 ([M+Na]⁺) calculated for C₃₃H₅₁N₃NaO₁₃P₂S⁺: 814.2510 (Δ0.6 ppm). 1H NMR (400 MHz, CDCl₃): δ=12.05 (s, 1H), 9.08 (d, J=2.3 Hz, 1H), 8.82 (d, J=2.3 Hz, 1H), 8.29 (dd, J=9.0, 2.7 Hz, 1H), 7.98 (dd, J=8.6, 2.3 Hz, 1H), 7.57 (d, J=9.0 Hz, 1H), 7.43 (d, J=9.0 Hz, 1H), 4.19 (q, J=7.4, 6.3, 5.9 Hz, 4H), 3.64 (t, J=6.3, 5.9 Hz, 4H), 1.51 (s, 36H) ppm. ¹³C NMR (101 MHz, CDCl₃, HSQC): δ=176.6, 144.4, 143.2, 142.0, 133.2, 131.3 (CH), 127.6 (CH), 127.2 (CH), 123.9 (CH), 120.8, 120.4, 119.0 (CH), 118.4 (CH), 83.4 (d, 4×C, J_{CP}=7.6 Hz), 65.3 (d, 2×CH₂, J_{CP}=6.9 Hz), 48.9 (d, CH₂, J_{CP}=8.4 Hz), 29.9 (d, 12×CH₃, J_{CP}=3.8 Hz) ppm. ³¹P NMR (162 MHz, CDCl₃): δ=−11.19 (s, 2P) ppm.

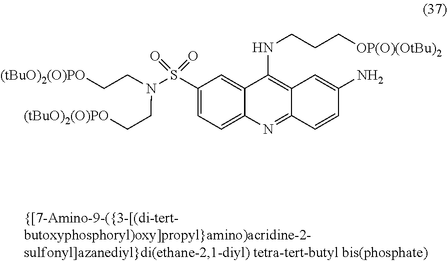

Compound 35 (58 mg, 56 μmol) was dissolved in MeOH (5 mL), containing AcOH (100 μL), under Ar. A 10% Pd/C catalyst (5 mg) was added, and hydrogen atmosphere was applied. After 1 h of stirring at rt, orange fluorescent solution was filtered through a pad of Celite and concentrated in vacuo. The residue was dissolved in 10 mL of ACN/0.1% aq. TFA (1:5) and purified by FC (C18, 30C18AQ-F0025 cartridge, ACN—0.1% aq. TFA). Good fractions were pooled and lyophilized to give a red powder (27 mg, 43%, TFA salt). HPLC: t_{R}=11.8 min (20-100); λₘₐₓ=475, 312, 275 nm. HRMS: m/z 1011.4449 ([M+H]⁺) calculated for C₄₄H₇₈N₄O₁₄P₃S⁺: 1011.4443 (A 0.6 ppm). ¹H NMR (400 MHz, MeOD-d₃): δ=8.95 (d, J=2.0 Hz, 1H), 8.17 (dd, J=9.0, 2.0 Hz, 1H), 7.86 (d, J=9.0 Hz, 1H), 7.66 (dd, J=8.2, 1.6 Hz, 1H), 7.51 (s, 1H), 7.49 (dd, J=9.0, 2.3 Hz, 1H), 4.30 (t, J=7.0, 6.7 Hz, 2H), 4.18 (q, J=6.3, 5.9 Hz, 2H), 4.10 (q, J=6.7, 5.9 Hz, 4H), 3.62 (t, J=5.9 Hz, 4H), 2.35 (quin, J=6.3, 5.9 Hz, 2H), 1.44 (s, 36H), 1.42 (s, 18H) ppm. ³¹P NMR (162 MHz, CDCl₃): δ=−10.02 (s, 2P), −10.86 (s, 1P) ppm.

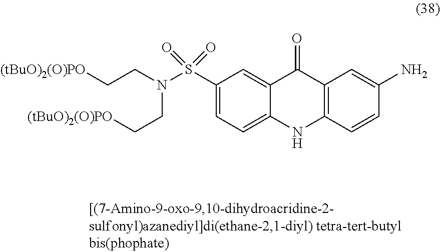

Compound 36 (12 mg, 15 μmol) was dissolved in MeOH (1 mL), containing AcOH (25 μL), under Ar. A 10% Pd/C catalyst (1 mg) was added, and hydrogen atmosphere was applied. After 1 h of stirring at rt, the reaction mixture was filtered through a pad of Celite and concentrated in vacuo. The residue was dissolved in 10 mL of ACN/0.1% aq. TFA (1:5) and purified by FC (C18, 30C18AQ-F0025 cartridge, ACN—0.1% aq. TFA). Good fractions were pooled and lyophilized to give a light yellow powder (6.3 mg, 77%, TFA salt). HPLC: t_{R}=9.6 min (20-100); λₘₐₓ=397, 380, 318, 306, 280, 256 nm. ESI-MS: m/z 762.4 [M+H]⁺. HRMS: m/z 762.2945 ([M+H]⁺) calculated for C₃₃H₅₄N₃O₁₁P₂S⁺: 762.2949 (Δ0.5 ppm). ¹H NMR (400 MHz, MeOD-d₃): δ=8.78 (d, J=2.0 Hz, 1H), 8.07-8.13 (m, 2H), 7.66 (d, J=8.6 Hz, 1H), 7.62 (d, J=1.6 Hz, 2H), 4.09 (q, J=7.0, 6.3, 5.9 Hz, 4H), 3.56 (t, J=5.9 Hz, 4H), 1.45 (s, 36H) ppm. ¹³C NMR (100 MHz, MeOD-d₃, HSQC): δ=177.2, 143.1, 138.6, 132.1, 131.2, 130.9 (CH), 127.5 (CH), 126.8 (CH), 121.6, 119.3 (CH), 119.2, 118.8 (CH), 116.2 (CH), 83.3 (d, 2×C, J_{CP}=7.6 Hz), 65.3 (d, 2×CH₂, J_{CP}=6.9 Hz), 48.7 (d, 2×CH₂, J_{CP}=8.4 Hz), 28.7 (d, 12×CH₃, J_{CP}=3.8 Hz) ppm. ³¹P NMR (162 MHz, MeOD-d₃): δ=−10.79 (s) ppm.

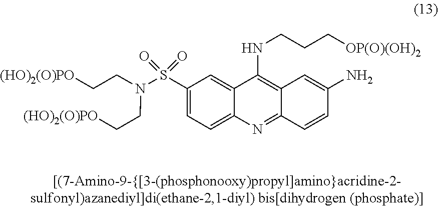

Compound 37 (11 mg, 10 μmol) was stirred in 5% TFA in DCM (0.75 mL) for 2 h at rt under Ar. The reaction mixture was then cooled to 0° C., and 5 mL of TEAAc buffer (1.0 M, pH 7) was added. The resulting emulsion was concentrated in vacuo (without heating bath) to obtain clear aqueous solution, which was purified by FC (C18, 30C18AQ-F0025, ACN—10 mM TEAAc pH 7). Good fractions were pooled and lyophilized (3×) to give a dark red solid (8 mg, 72%, TEA salt). HPLC (Knauer Eurospher II 100-5 C18A): t_{R}=8.7 min (TEAB-0-25); t_{R}=7.0 min (TEAB-0-40); λₘₐₓ=458, 305, 273 nm. ESI-MS: m/z 673.2 [M−H]⁻. HRMS: m/z 673.0532 ([M−H]⁻) calculated for C₂₀H₂₈N₄O₁₄P₃S⁻: 673.0541 (Δ1.3 ppm). 1H NMR (400 MHz, D₂O): 6=8.46 (s, 1H), 8.05 (d, J=9.0 Hz, 1H), 7.63 (d, J=9.4 Hz, 1H), 7.30 (d, J=8.6 Hz, 1H), 7.25 (d, J=9.0 Hz, 1H), 7.16 (s, 1H), 4.08-4.20 (m, 2H), 4.04 (t, J=6.3, 5.9 Hz, 2H), 3.98 (q, J=5.9, 5.5, 5.1 Hz, 4H), 3.58 (t, J=5.5, 5.1 Hz, 4H), 3.19 (q, J=7.4 Hz, 26H, TEA), 2.12-2.26 (m, 2H), 1.26 (t, J=7.4 Hz, 38H, TEA) ppm. ³¹P NMR (162 MHz, D₂O): δ=1.32 (s, 1P), 0.72 (s, 2P) ppm.

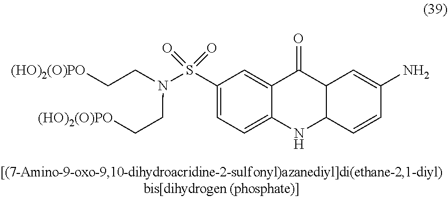

Compound 38 (6.3 mg, 7 μmol) was stirred in 5% TFA in DCM (0.75 mL) for 2 h at rt under Ar. The reaction mixture was then cooled to 0° C., and 5 mL of TEAAc buffer (1.0 M, pH 7) was added. The resulting emulsion was concentrated in vacuo (without heating bath) to obtain clear aqueous solution, which was purified by FC (C18, 30C18AQ-F0025, ACN—10 mM TEAAc pH 7). Good fractions were pooled and lyophilized (3×) to give a yellow-brown solid (4 mg, 79%, TEA salt). HPLC (Knauer Eurospher II 100-5 C18A): t_{R}=8.6 min (TEAB-0-40); λₘₐₓ=425, 297, 260 nm. ESI-MS: m/z 536.1 [M−H]⁻. HRMS: m/z 536.0298 ([M−H]⁻) calculated for C₁₇H₂₀N₃O₁₁P2S 536.0299 (Δ0.2 ppm). 1H NMR (400 MHz, D₂O): δ=8.32 (d, J=2.0 Hz, 1H), 7.84 (dd, J=9.0, 1.2 Hz, 1H), 7.27 (d, J=9.0 Hz, 1H), 7.23 (s, 1H), 7.18 (dd, J=8.6, 0.8 Hz, 1H), 7.06 (d, J=9.0 Hz, 1H), 4.00 (q, J=5.9, 5.5 Hz, 4H), 3.51 (t, J=5.5, 5.1 Hz, 4H), 3.17 (q, J=7.4 Hz, TEA), 1.25 (t, J=7.4 Hz, TEA) ppm. ¹³C NMR (101 MHz, D₂O, HSQC): 6=177.5, 141.7, 139.1, 135.2, 130.0 (CH), 129.6, 126.8 (CH), 126.6 (CH), 120.6, 119.1 (CH), 119.0 (CH), 117.4, 110.1 (CH), 63.5 (d, 2×CH₂, J_{CP}=4.6 Hz), 49.1 (d, 2×CH₂, J_{CP}=6.9 Hz), 46.7 (CH₂, TEA), 8.3 (CH₃, TEA) ppm. ³¹P NMR (162 MHz, D₂O): δ=0.28 (s) ppm.

### Example 4

### Reductive Amination of Carbohydrates with Negatively Charged Amino-Dyes

General method for labelling of carbohydrates with negatively charged amino-dyes for electrophoresis. 1.5 mL Brand® micro tube with a screw cap was charged with dye (1 equiv., 0.1 M solution in water), carbohydrate (1 equiv., 0.1 M solution in water), malonic acid (10 equiv., 1 M solution in DMSO) and 2-picoline-borane complex (10 equiv., 1 M solution in DMSO). After vortexing for 10 s, the reaction mixture was incubated in an Eppendorf ThermoMixer® with shaking (400-600 rpm) at 40° C. for 18 h. The reaction mixture was cooled to rt, diluted with 5 mL of TEAB buffer (1.0 M, pH 8) and purified by flash chromatography (RP C18, 15C18AQ-F0025 cartridge, ACN—20 mM TEAB, pH 8, 0-5% ACN, 10 column volumes). The appropriate fractions were pooled and lyophilized.

Removal of buffer at reduced pressure and prolonged drying gave the conjugation products of dye 13 as red powders. These compounds did not dissolve any more in water, methanol, ethanol, DMSO, or DMF even on heating.

General method for labelling of dextran ladder with negatively charged amino-dyes for electrophoresis. 1.5 mL Brand® micro tube with a screw cap was charged with a dextran ladder (0.4 mg, maltodextrin oligosaccharides—DP2 to DP15, Carbosynth), dye (5 μL, 0.1 M solution in water or DMSO), malonic acid (5 μL, 1 M solution in DMSO) and 2-picoline-borane complex (5 μL, 1 M solution in DMSO). After vortexing for 10 s, the reaction mixture was incubated in an Eppendorf ThermoMixer® with shaking (400-600 rpm) at 40° C. for 18 h. The reaction mixture was cooled to rt, diluted with 5 mL of TEAB buffer (1.0 M, pH 8) and purified by flash chromatography (RP C18, 15C18AQ-F0025 cartridge, ACN—20 mM TEAB, pH 8, 0-5% ACN, 10 column volumes). The appropriate fractions were pooled and concentrated in vacuo (rotary evaporator, then speedvac).

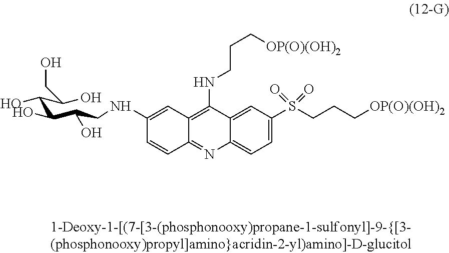

HPLC (Knauer Eurospher II 100-5 C18A): t_{R}=9.5 min (TEAB-0-25); λₘₐₓ=469, 317, 276 nm. ESI-MS: m/z 712.4 [M−H]⁻. HRMS: m/z 712.1339 ([M−H]⁻) calculated for C₂₅H₃₆N₃O₁₅P₂S⁻: 712.1348 (Δ1.3 ppm). ¹H NMR (400 MHz, D₂O): δ=8.60 (s, 1H), 8.04 (d, J=8.6 Hz, 1H), 7.69 (d, J=9.4 Hz, 1H), 7.39 (d, J=9.0 Hz, 1H), 7.16-7.35 (m, 1H), 6.91 (s, 1H), 4.06-4.17 (m, 4H), 4.04 (quin, J=5.5, 4.3, 3.5 Hz, 1H), 3.86-3.96 (m, 3H), 3.78-3.86 (m, 2H), 3.74-3.79 (m, 1H), 3.64-3.73 (m, 1H), 3.49-3.61 (m, 2H), 3.40 (dd, J=13.5, 3.7 Hz, 1H), 3.23 (dd, J=13.7, 8.2 Hz, 1H), 3.18 (q, J=7.4 Hz, TEA), 2.17-2.29 (m, 2H), 1.97-2.08 (m, 2H), 1.25 (t, J=7.4 Hz, TEA) ppm. ³¹P NMR (162 MHz, D₂O): δ=0.69 (s, 1P), 0.33 (s, 1P) ppm.

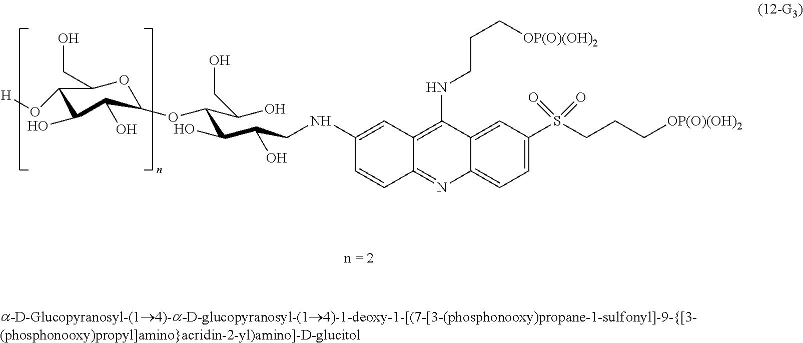

HPLC (Knauer Eurospher II 100-5 C18A): t_{R}=8.8 min (TEAB-0-25). ESI-MS: m/z 1036.6 [M−H]⁻.

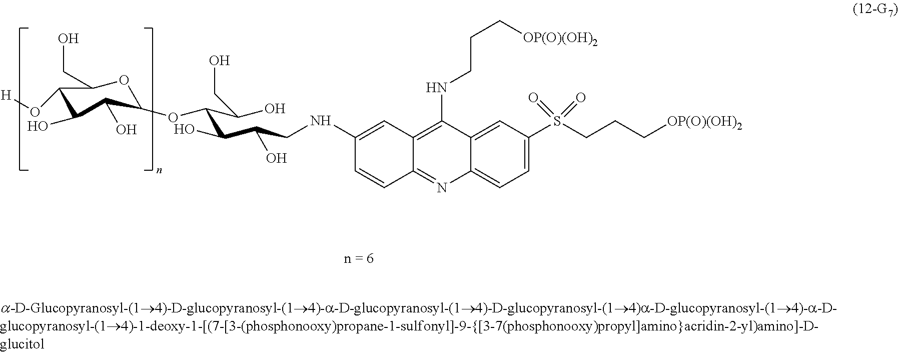

HPLC (Knauer Eurospher II 100-5 C18A): t_{R}=8.8 min (TEAB-0-25). ESI-MS: m/z 1685.6 [M−H]⁻.

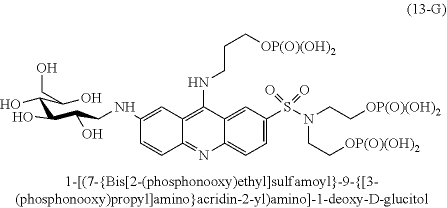

HPLC (Knauer Eurospher II 100-5 C18A): t_{R}=8.8 min (TEAB-0-25); λₘₐₓ=473, 318, 276 nm. ESI-MS: m/z 836.9 [M−H]⁻. HRMS: m/z 418.0568 ([M−2H]²⁻) calculated for C₂₆H₃₉N₄O₁₉P₃S²: 418.0577 (z 2.2 ppm).

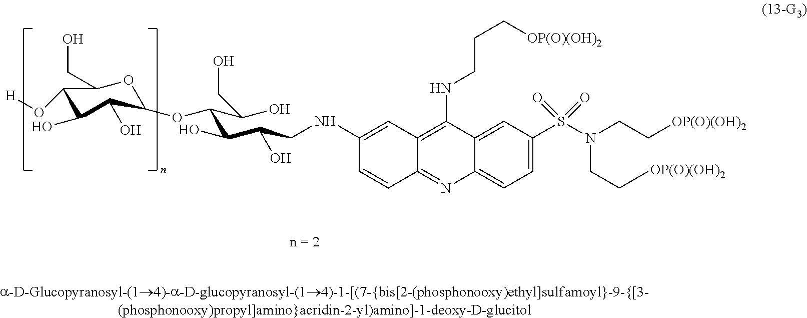

HPLC (Knauer Eurospher II 100-5 C18A): t_{R}=9.0 min (TEAB-0-25). ESI-MS: m/z 1161.1 [M−H]⁻.

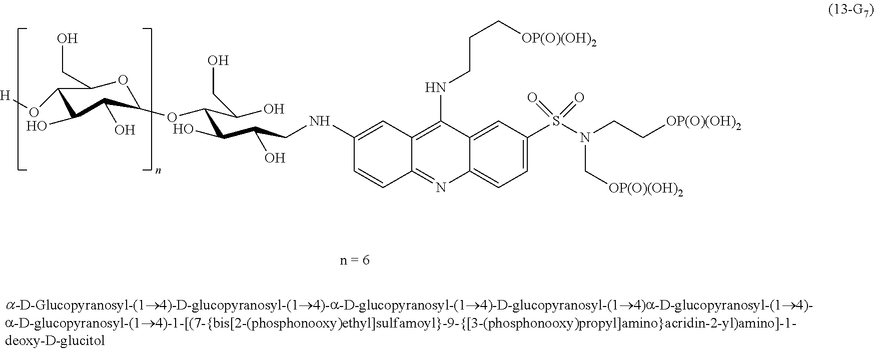

HPLC (Knauer Eurospher II 100-5 C18A): t_{R}=8.8 min (TEAB-0-25). ESI-MS: m/z 904.1 [M−2H]²⁻.

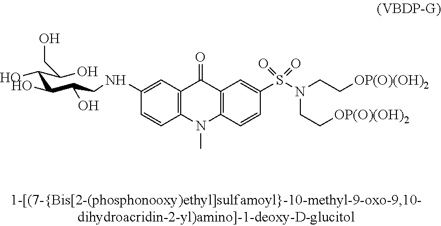

HPLC (Knauer Eurospher 11100-5 C18A): t_{R}=10.6 min (TEAB-0-25); λₘₐₓ=439, 306, 254 nm. ESI-MS: m/z 714.2 [M−H]⁻. HRMS: m/z 356.5533 ([M−2H]²⁻) calculated for C₂₄H₃₃N₃O₁₆P₂S²⁻: 356.5534 (Δ0.3 ppm).

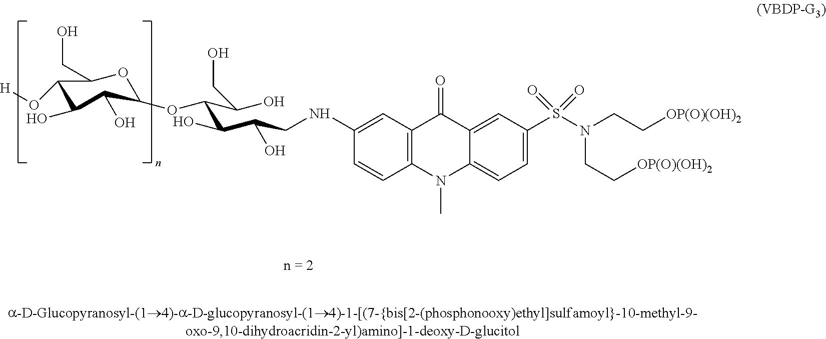

HPLC (Knauer Eurospher II 100-5 C18A): t_{R}=10.2 min (TEAB-0-25). ESI-MS: m/z 1038.4 [M−H]⁻.

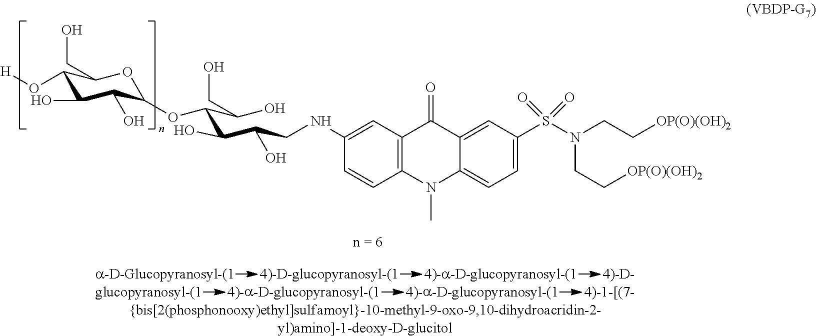

HPLC (Knauer Eurospher II 100-5 C18A): t_{R}=10.3 min (TEAB-0-25). ESI-MS: m/z 1686.6 [M−H]⁻.

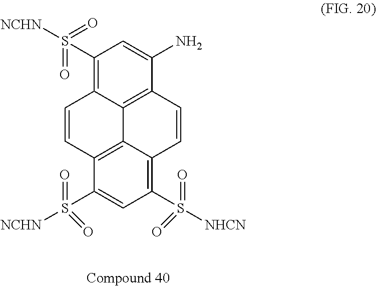

a) Anhydrous trisodium salt of 8-aminopyrene-1,3,6-trisulfonic acid (52 mg, 0.10 mmol) was introduced into a 10 mL flask, cooled down to 0° C. (ice bath), and chlorosulfonic acid (356 mg, 204 μL, 3 mmol) was added dropwise with stirring. The reaction mixture was stirred at r. t. for 4 h. After cooling down to 0° C., the reaction mixture was carefully transferred onto crushed ice (50 g). The red precipitate of trisulfonyl chloride was isolated by centrifugation and washed with ice water (3×50 mL; with centrifugation). The crude compound was lyophilized to afford 42 mg of 1,3,6-tri(chlorosulfonyl)-8-aminopyrene (49) (82% yield). The flask was purged with Ar and kept in the freezer (−20° C.).

b) To a stirred and ice-cooled solution of cyanamide (50 mg, 1.2 mmol) and triethylamine (122 mg, 176 μL, 1.2 mmol) in aqueous MeCN (1:1, 1.5 mL), the solid sulfonylchloride (21 mg, 0.04 mmol) was added in portions. The reaction mixture turned orange. When the addition was complete, the reaction mixture was stirred for 10 min, diluted with aq. TEAB (Et3N*H₂CO₃, 2 mL, pH=8), frozen and lyophilized. Analytical HPLC: Kinetex, 5 μm C18 100, 250 mm, 4.6 mm, ACN/0.05 M TEAB: 5/95-60/40 in 20 min, 1.2 mL/min; t_{R}=9.4 min. The title compound was isolated by preparative IPLC with UV-VIS detection (MeCN/TEAB 0.05 M in water, 5:95→30:70 in 20 min detected at 500 nm)

¹H NMR (400 MHz, D₂O) δ 9.14 (s, 1H), 8.84 (d, J=9.7 Hz, 1H), 8.57 (d, J=9.6 Hz, 1H), 8.52 (d, J=9.7 Hz, 1H), 8.03 (d, J=9.6 Hz, 1H), 7.99 (s, 1H), 3.20 (d, J=7.2 Hz, 1H), 2.82 (q, J=7.3 Hz, 18H, CH₂ in Et₃N), 0.99 (t, J=7.3 Hz, 27H, CH₃ in Et₃N). ¹³C NMR (101 MHz, D₂O) δ 144.8, 138.2, 131.3, 131.1, 130.8, 130.4, 128.2, 126.4, 125.5, 124.8, 124.7, 122.5, 120.1, 116.6, 116.0, 115.4, 46.4, 8.0.

HR-MS: C₁₉H₁₁N₇O₆S₃ found 527.9850 [M−H]⁻; calculated 527.9860.

λₘₐₓ (absorption)=454 nm (H₂O), F=23 900 M⁻¹ cm⁻¹, λₘₐₓ (emission)=531 nm (excitation at 440 nm); Stocks shift 75 nm, fluorescence lifetime 5.6 ns (H₂O; excitation at 440 nm); fluorescence quantum yield: 0.93 (H₂O). See FIG. 23.

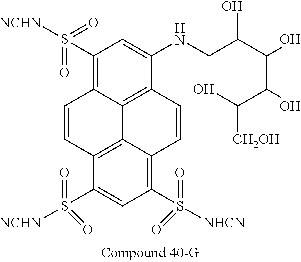

A 1.5 mL Eppendorf vial was charged with dye 40 (100 μL of 0.02 M solution in water), glucose (5 equiv., 10 μmol, 2 mg), and malonic acid (10 equiv., 20 μL of 1 M solution in DMSO). The closed vial shaken at 40° C. for 1 h (Eppendorf ThermoMixer®), and then the solvents (water and DMSO) were removed by lyophilization (p<0.2 mbar). A solution of 2-picoline-borane complex (10 equiv., 20 μL of 1 M solution in DMSO) was added, and the sample was shaken at 40° C. for 16 hours (Eppendorf ThermoMixer®). The product was isolated by preparative HPLC with UV-VIS detection (MeCN/TEAB 0.05 M in water, 5:95→30:70 in 20 min, detected at 500 nm). The product was characterized by ESI-HRMS, UV-Vis and fluorescence spectroscopy.

HR-MS: C₂₅H₂₃N₇O₁₁S₃ found 692.9850 [M−H]⁻; calculated 692.9860.

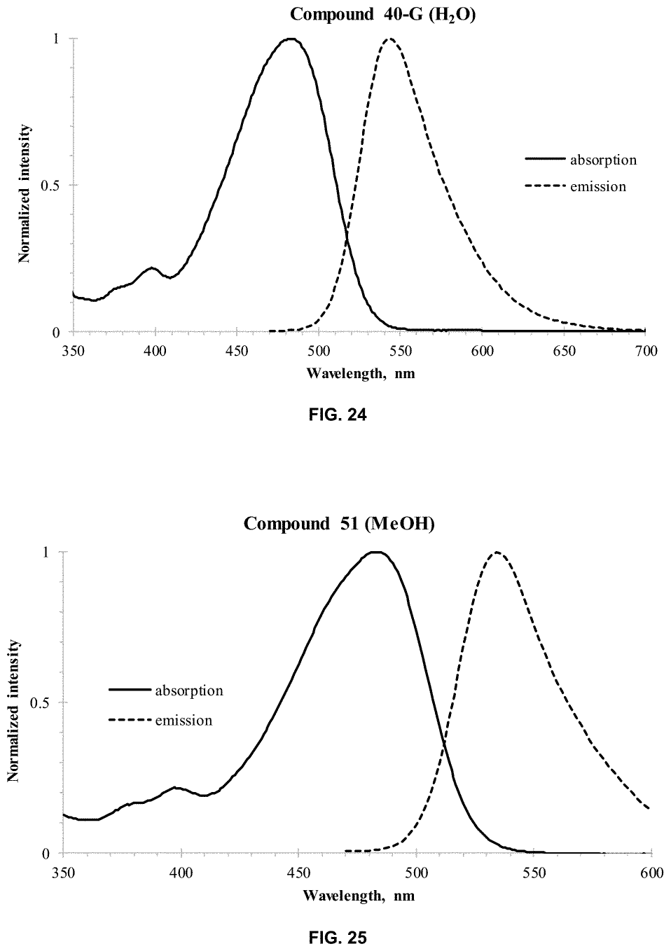

λₘₐₓ (absorption)=483 nm (H₂O), λₘₐₓ (emission)=544 nm (excitation at 460 nm); Stocks shift 61 nm, fluorescence lifetime 5.34 ns (H₂O; excitation at 440 nm); fluorescence quantum yield: 0.92 (H₂O). See FIG. 24.

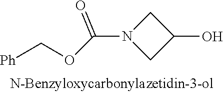 [SMILES: O=C(OCc1ccccc1)N1CC(O)C1]

N-enzyloxycarbonylazetidini-3-ol was synthesized according to the literature (T. A. Davis, M. W. Danneman, J. N. Johnston. Chem. Comm. 2012, 48, 5578-5580).

¹H NMR (400 MHz, Acetone-d₆) δ 7.47-7.24 (m, 5H), 5.06 (d, J=0.7 Hz, 2H), 4.62-4.53 (m, 1H), 4.15 (s, 2H), 3.84-3.68 (m, 2H).

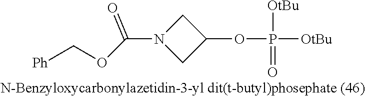 [SMILES: CC(C)(C)OP(=O)(OC1CN(C(=O)OCc2ccccc2)C1)OC(C)(C)C]

1H-Tetrazole (840 mg, 12 mmol) and di-t-butyl N,N-diisopropylphosphoramidite (2.8 g, 3.18 mL, 9.6 mmol) were added to a solution of N-benzyloxycarbonylazetidin-3-ol (1.0 g, 4.8 mmol) in DMF (8 mL) under Ar, and the mixture was stirred at r.t. for 1 h. The course of the reaction was monitored by HPLC. After the starting compound disappeared, the mixture was cooled to 0° C., and H₂O₂ (70% aqueous solution, 1.5 mL) was added. After 15 min the cooling bath was removed, the reaction mixture analyzed by HPLC, and then aqueous Na₂SO₃ (10%, 30 mL) was added (with cooling of the reaction mixture in an ice bath). After 30 min, the reaction mixture was extracted with EtOAc (30 mL×3). The combined solutions were washed with brine, dried over MgSO₄, and evaporated. The residue was submitted to flash chromatography (SNAP Ultra cartridge with 50 g SiO₂, hexane/EtOAc with 20-65% EtOAc-gradient over 15 CV) to provide a white solid (1.2 g, 62% yield).

HR-MS: C₁₉H₃₀NO₆P found 400.1882 [M+H]⁺, calculated 400.1884; found 422.1703 [M+Na]⁺, calculated 422.1703.

¹H NMR (400 MHz, Acetone-d₆) δ 7.41-7.28 (m, 5H), 5.08 (s, 2H), 5.06-4.98 (m, 1H), 4.33-4.23 (m, 2H), 4.06-3.97 (m, 2H), 1.47 (d, J=0.7 Hz, 18H).

¹³C NMR (101 MHz, Acetone-d₆) δ 138.0, 129.2, 128.7, 128.6, 83.2, 83.1, 66.9, 66.0, 65.9, 30.0. ³¹P NMR (162 MHz, Acetone-d₆) δ−10.93.

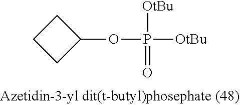 [SMILES: CC(C)(C)OP(=O)(OC1CCC1)OC(C)(C)C]

A solution of N-benzyloxycarbonylazetidin-3-yl di(t-butyl)phosphate (46) (640 mg, 1.6 mmol) in MeOH (10 mL) was added to a Schlenk-flask charged with Pd/C (10% Pd in an oxidized form, 160 mg) in THE (3 mL), which was pre-reduced with H₂. The reaction mixture was stirred overnight at r.t. under hydrogen. The reaction mixture was flushed with argon, transferred into centrifuge tubes, the catalyst removed, and washed with THF. The supernatant was evaporated to give a colorless oil (390 mg, 92% yield).

HR-MS: C₁₁H₂₄NO₄P, found 266.1504 [M+H]⁺, calculated 266.1516; found 288.1346 [M+Na]⁺, calculated 288.1335.

¹H NMR (400 MHz, Acetonitrile-d₃) δ 5.03-4.94 (m, 1H), 4.26-4.18 (m, 2H), 4.11-4.01 (m, 2H), 1.41 (d, J=0.7 Hz, 18H).

¹³C NMR (101 MHz, Acetonitrile-d₃) δ 85.6, 85.5, 67.2, 65.2, 55.3, 54.6, 30.0, 29.9, 1.9, 1.7.

³¹P NMR (162 MHz, Acetonitrile-d₃) δ −12.02.

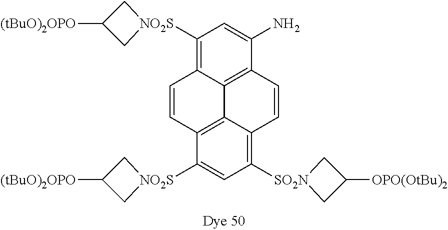

A solid sulfonyl chloride 49 (0.02 mmol, 11 mg) was added in portions to a stirred and cooled (0° C.) solution of azetidin-3-yl di(t-butyl)phosphate 48 (0.2 mmol, 53 mg) and Et₃N (52 μL, 0.4 mmol) in MeCN (2 mL). After stirring for 10 min, the reaction mixture was analyzed by TLC (DCM/MeOH, 10/1), and a spot with R_{f}=0.5 was detected. The reaction mixture was lyophilized, and the residue was submitted to flash chromatography (SNAP Ultra cartridge with 10 g SiO₂, gradient of DCM/MeOH with 2-15% MeOH over 15 CV) to provide an orange solid (2.0 mg, 8% yield).

HR-MS: C₄₉H₇₇N₄O₁₈P₃S₃, found 1199.3645 [M+H]⁺, calculated 1199.3681; found 1221.3505 [M+Na]⁺, calculated 1221.3500

¹H NMR (400 MHz, Acetonitrile-d₃) δ 9.24 (d, J=9.8, 0.6 Hz, 1H), 9.09 (d, J=0.6 Hz, 1H), 9.05 (d, J=9.7, 0.6 Hz, 1H), 8.89 (d, J=9.8, 0.6 Hz, 1H), 8.60 (d, J=9.7 Hz, 1H), 8.24 (d, J=0.6 Hz, 1H), 6.18 (s, 2H), 4.84-4.72 (m, 3H), 4.26-4.10 (m, 6H), 4.01-3.84 (m, 6H), 1.29 (s, 18H), 1.29 (s, 18H), 1.24 (s, 18H).

³¹P NMR (162 MHz, Acetonitrile-d₃) 6-11.63 (“d”, J=4.3 Hz), −11.75.

### Example 5

### Synthesis of Fluorescent 1-Aminopyrene Dyes and their Precursors

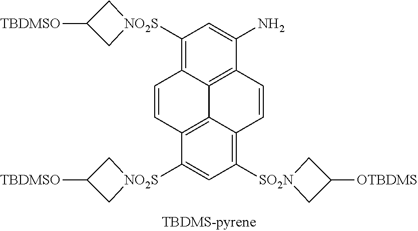

A solution of sulfonyl chloride 49 (0.2 mmol, 110 mg) in 3.0 mL of ACN was added dropwise to a stirred and cooled (0° C.) solution of 3-(tet-butyldimethylsilyloxy)azetidine (1.35 mmol, 253 mg) and Et₃N (176 μL, 1.35 mmol) in MeCN (1 mL). After stirring for 1 h, the reaction mixture was analyzed by TLC (DCM/MeOH, 10/1), and a spot with R_{f}=0.98 was detected. The reaction mixture was lyophilized, the residue submitted to flash chromatography (SNAP Ultra cartridge with 25 g SiO₂, gradient of DCM/MeOH with 1-10% MeOH over 15 CV) to provide an orange solid (48 mg, 25% yield).

HR-MS: C₄₃H₆₈N₄O₉S₃Si₃, found 965.3522 [M+H]⁺, calculated 965.3529; found 987.3341 [M+Na]⁺, calculated 987.3348

¹H NMR (400 MHz, Acetonitrile-d₃) δ 9.24 (d, J=9.8 Hz, 1H), 9.08 (s, 1H), 9.06 (d, J=9.6 Hz, 1H), 8.90 (d, J=9.7 Hz, 1H), 8.57 (d, J=9.7 Hz, 1H), 8.22 (s, 1H), 6.11 (s, 2H), 4.55-4.43 (m, 3H), 4.11-4.01 (m, 6H), 3.75-3.62 (m, 6H), 0.67 (d, J=0.7 Hz, 18H), 0.61 (s, 10H), −0.09 (d, J=0.5 Hz, 12H), −0.13 (s, 6H).

¹³C NMR (126 MHz, Acetonitrile-d₃) δ 147.7, 135.4, 134.4, 131.9, 130.3, 128.0, 127.3, 127.2, 127.1, 126.6, 124.2, 121.4, 119.5, 61.5, 61.4, 61.3, 61.2, 25.8, 25.7, 18.3, −5.0, −5.05, −5.1.

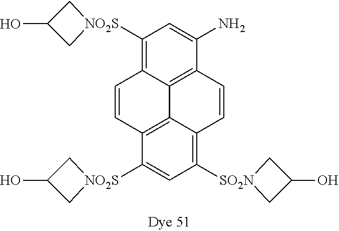

The reaction was carried out in a plastic test-tube (with a screw-cap) used for centrifugation. To a stirred solution of TBDMS-pyrene (44 mg, 0.45 mmol) in MeCN, 50-55% aq. HF (74 μL, 2.33 mmol) was added at 0° C. After stirring for 4 h, an additional amount of HF was added (74 μL, 2.33 mmol), and the reaction mixture was stirred overnight at r.t. The reaction mixture was analyzed by TLC (DCM/MeOH, 10/1), a new spot with R_{f}=0.25 was detected. The reaction was “quenched” by addition of an aqueous sodium bicarbonate solution (5%, 10 mL) and brine. The product was extracted with DCM/iPrOH (1:1; 3×100 mL). The combined organic solutions were dried over MgSO₄ and concentrated in vacuo. The residue was submitted to flash chromatography (SNAP Ultra cartridge with 25 g SiO₂, gradient of DCM/MeOH with 2-20% MeOH over 15 CV) to provide an orange solid (27 mg, 96%).

HR-MS: C₂₅H₂₆N₄O₉S₃, found 623.0913 [M+H]⁺, calculated 623.0935; found 621.0773 [M−H]⁻, calculated 624.0789.

¹H NMR (400 MHz, DMSO-d₆) δ 9.06 (d, J=9.7 Hz, 1H), 8.91 (s, 1H), 8.87 (d, J=9.7 Hz, 1H), 8.83 (d, J=9.7 Hz, 1H), 8.70 (d, J=9.8 Hz, 1H), 8.16 (s, 1H), 7.70 (s, 2H), 5.72-5.59 (m, 3H), 4.38-4.24 (m, 3H), 4.07-3.91 (m, 6H), 3.65-3.45 (m, 6H).

¹³C NMR (101 MHz, DMSO-d₆) δ 148.2, 134.3, 133.3, 133.1, 130.6, 129.1, 127.1, 126.6, 125.9, 125.0, 124.0, 121.9, 119.1, 117.1, 116.7, 115.8, 60.2, 60.0, 58.6, 58.5, 40.4.

λₘₐₓ (absorption)=483 nm (ε=19000 M⁻¹ cm⁻¹, MeOH), λₘₐₓ (emission)=534 nm (MeOH; excitation at 450 nm); Stocks shift 51 nm, fluorescence lifetime 5.6 ns (MeOH), fluorescence quantum yield: 0.77 (absolute value in MeOH). See FIG. 25.

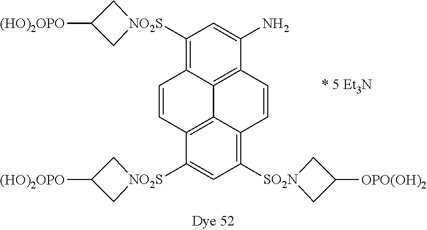

Ester 50 (2.0 mg, 1.67 μmol) was dissolved in dichloromethane (0.5 mL), the solution cooled to +5° C. in an ice bath, and then a solution of TFA in DCM (5% v/v, 0.5 mL) was added slowly with stirring. The reaction mixture was allowed to warm-up to r. t. and stirred for 2 h. The volatile materials were removed in vacuo. The residue was treated with 1 M aq. Et₃N*H₂CO₃ buffer (TEAB; pH=8-9) and stirred, until the pH stabilized at 8-9. The title compound was isolated by preparative HPLC (Kinetex 5 μm EVO C18 100A 250×21 mm column, MeCN/water+0.05 M TEAB, 10 mL/min, 5-30% MeCN over 20 min) to afford 2 mg of dye 52 as red solid (88% yield of pentakis triethylammonium salt, according to ¹H-NMR).

HR-MS: C₂₅H₂₉N₄O₁₈P₃S₃, found 860.9760 [M−H]⁻, calculated 860.9779.

¹H NMR (400 MHz, D₂O) δ 9.05 (s, 1H), 8.97 (d, J=9.8 Hz, 1H), 8.72 (d, 1H), 8.63 (d, J=7.0 Hz, 1H), 8.44 (d, J=16.6 Hz, 1H), 8.14 (s, 1H), 4.23-4.13 (m, 6H), 4.04-3.89 (m, 6H), 3.17 (q, 30H), 1.26 (t, J=7.3, 0.6 Hz, 45H).

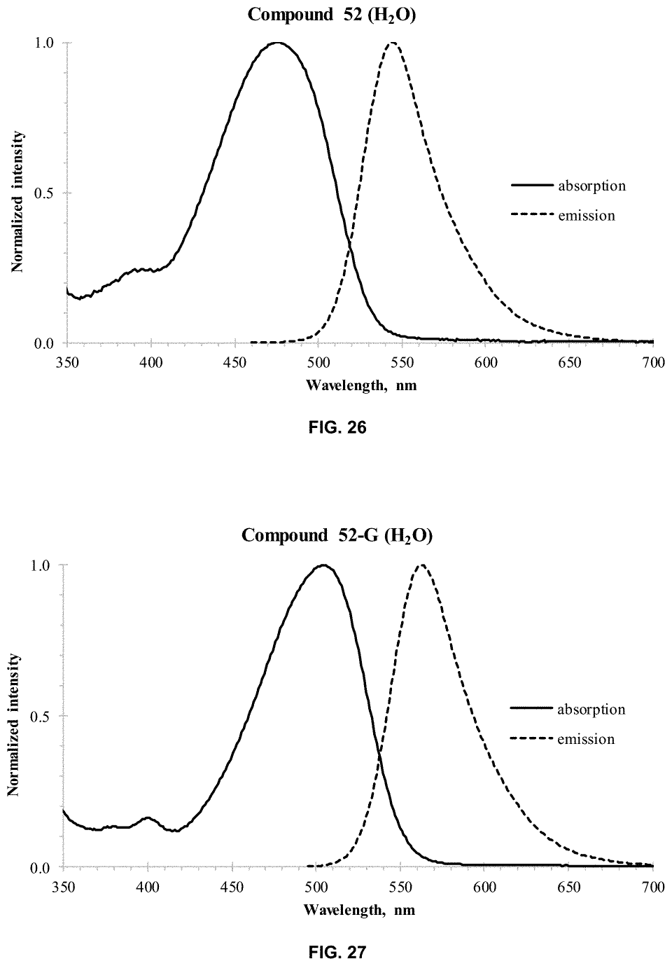

λₘₐₓ (absorption)=476 nm (H₂O), λₘₐₓ (emission)=543 nm (H₂O; excitation at 450 nm); Stocks shift 67 nm, fluorescence lifetime 5.9 ns (H₂O), fluorescence quantum yield: 0.8 (absolute value in H₂O). See FIG. 26.

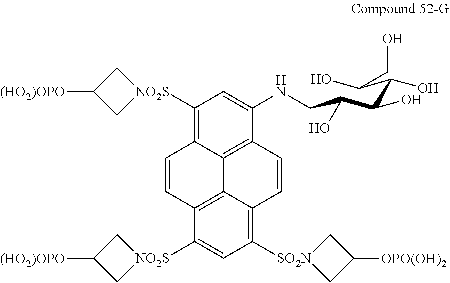

A 1.5 mL Eppendorf vial was charged with dye 52 (2 mg, 1.5 μmol), glucose (5 equiv., 7.5 μmol, 1.5 mg) and malonic acid (10 equiv., 15 μL of 1 M solution in DMSO). The sample was stirred at 40° C. for 1 h (Eppendorf ThermoMixer®), and then water and DMSO were removed under reduced pressure (p<0.2 mbar) in lyophilizer. A solution of 2-picoline-borane complex (10 equiv., 15 μL of 1 M solution in DMSO) was added to the residue, and the samples were shaken at 40° C. for 16 h (Eppendorf ThermoMixer®). The product was isolated by preparative HPLC with UV-VIS detection (MeCN/TEAB 0.05 M in water, 5:95→30:70 in 20 min, detected at 500 nm).

HR-MS: C₃₁H₄₁N₄O₂₃P₃S₃, found 1025.3522 [M−H]⁻, calculated 1025.3529.

λₘₐₓ (absorption)=505 nm (H₂O), λₘₐₓ (emission)=565 nm (H₂O; excitation at 485 nm); Stocks shift 60 nm, fluorescence lifetime 5.8 ns (H₂O), fluorescence quantum yield: 0.85 (absolute value in H₂O). See FIG. 27.

### Example 6

### Conjugation of Aminopyrene Dyes with Sugars (“Sugar Ladders”)

General Procedure:

1.5 mL Eppendorf vial was charged with a dye (compound 40 or APTS, 10 μL of 0.1 M solution in water), a dextran ladder (1.0 mg, maltodextrin oligosaccharides—DP2 to DP15, Carbosynth), and malonic acid (10 equiv., 10 μL of 1 M solution in DMSO). The samples were shaken at 40° C. for 1 h (Eppendorf ThermoMixer®), and then solvents were removed under reduced pressure (p<0.2 mbar) in lyophilizer. A solution of 2-picoline-borane complex (10 equiv., 10 μL of 1 M solution in DMSO) was added, and the samples were shaken at 40° C. for 16 h (Eppendorf ThermoMixer®). The products were isolated by preparative HPLC with UV-VIS detection (MeCN/aq. TEAB 5:95→30:70 in 20 min detected at 500 nm) or FC (C18, 15C18AQ-F0025 multi-use cartridge, MeCN/aq. TEAB 5:95→30:70).

Analytical HPLC: Kinetex, 5 μm C18 100, 25 cm×4.6 mm, MeCN/aq. TEAB 5:95→30:70 in 20 min detected at 500 nm, 1.2 mL/min;

Preparative HPLC: Kinetex, 5 μm C18 100, 25 cm×10 mm, MeCN/aq. TEAB 5:95→30:70 in 20 min detected at 500 nm, 4 mL/min;

Flash chromatography: RP (C18) cartridge, 15C18AQ-F0025 (Interchim), MeCN/aq. TEAB 5:95→30:70

1. A fluorescent dye having multiple ionizable and/or negatively charged groups which is selected from the group consisting of compounds of the following general formulae A, B, C, D, E, including salts or protonated forms thereof: 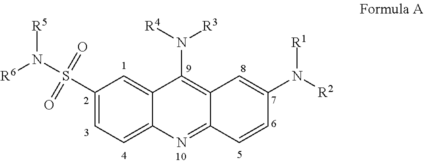
wherein
R¹, R², R³, R⁴, R⁵, R⁶ are independent from each other and selected from the group consisting of
a) hydrogen;
b) CH₃, C₂H₅, a straight or branched C₃-C₆ alkyl, deuterated alkyl, or perfluoroalkyl group;
c) straight or branched C₂-C₆ alkyl group that is substituted with 1 to 3 halogen atoms, or incorporates 1 to 3 heteroatoms selected from O, S, and N, or that is optionally substituted with solubilizing groups selected from OH, NH₂, SH, and COOH and/or ionizable anionic residues selected from the group consisting of (alkyl) carboxy (CH₂)ₙCO₂H, (alkyl) sulfonate (CH₂)ₙSO₃H, (alkyl) sulfate (CH₂)ₙOSO₃H, (alkyl) phosphate (CH₂)ₙOP(O)(OH)₂ and (alkyl) phosphonate (CH₂)ₙP(O)(OH)₂, wherein n is an integer ranging from 0 to 6, or analogs thereof wherein one or more of the CH₂ groups are replaced by CF₂ and/or CD₂;
d) (CH₂)ₙCOOR⁷, where n=1-6, and R⁷ is alkyl CH₂CN, CH₂CF₃, benzyl, substituted benzyl, polyhaloalkyl, polyhalophenyl, 2- and 4-nitrophenyl, N-succinimidyl, sulfo-N-succinimidyl, 1-benzotriazolyl, 9-aza-1-benzotriazolyl or other forming an active ester of a carboxylic acid;
e) a carbonate or carbamate derivative (CH₂)ₘOCOOR⁸ or (CH₂)ₘNHCOOR⁸, where m=1-6 and R⁸═C₁-C₄ alkyl, benzyl, 9-fluorenylmethyl, CH₂CN, N-succinimidyl, sulfo-N-succinimidyl, 1-benzotriazolyl, phenyl, substituted phenyl group, 2-pyridyl, 4-pyridyl, or pyrimid-4-yl;
f) (CH₂)ₘNRₐR_{b}, where m=1-6, with a straight or branched alkyl chain; R^{a}, R^{b} are independent from each other and represent hydrogen, C₁-C₄ alkyl and/or C₁-C₄ acyl groups, optionally substituted with a hydroxyalkyl group (CH₂)ₙOH, where n=1-6, with a straight or branched alkyl chain, a phosphorylated hydroxyalkyl group (CH₂)ₚOP(O)(OH)₂, where p=1-6, with a straight or branched alkyl chain;
g) an alkyl azide (CH₂)_{q}N₃, where q=1-8, with a straight or branched alkyl chain;
h) an alkylalkyne (CH₂)ᵣC≡CR⁹, where r=1-8, with a straight or branched alkyl chain in (CH₂)ᵣ and R⁹=hydrogen, C₁-C₆ alkyl or (functionally) substituted a C₁-C₆ alkyl;
i) (CH₂)ₛCONHR¹⁰, with s=1-8, R¹⁰═H, C₁-C₆ alkyl, (CH₂)_{q}N₃, (CH₂)ᵣC≡CH or (CH₂)ₜ—N-maleimido, (CH₂)ᵤ—NH—COCH₂X with X═Br or I, where q, r, t, u indices are independent in the range 1-8, and with straight or branched alkyl chains in (CH₂)ₛ, (CH₂)_{q}, (CH₂)ᵣ, (CH₂)ₜ, (CH₂)ᵤ and R¹⁰;
j) a primary amino group (NH₂) or secondary amino group (NHR¹¹); R¹¹═C₁-C₆ alkyl or (functionally) substituted C₁-C₆ alkyl;
R¹, R², R³, R⁴, R⁵, R⁶ may contain a terminal alkyloxyamino group (CH₂)ᵥONH₂, where v=1-12 with a straight or branched alkyl chain that can include one or multiple (alkyl)oxy (CH₂)ₖO, (alkyl)amino (CH₂)ₗNH or (alkyl)amido (CH₂)_{w}CONH groups in all possible combinations with indices k, l and w=0-12 (independent from each other;
R¹, R², R³, R⁴, R⁵, R⁶ may contain a terminal acylhydrazino group (CH₂)ₓCONHNH₂, where x=1-12, with a straight or branched alkyl chain that can include one or multiple (alkyl)oxy (CH₂)ₖO, (alkyl)amino (CH₂)ₗNH or (alkyl)amido (CH₂)_{w}CONH groups in all possible combinations with indices k, l and w=0-12 independent from each other;
one of the residues R¹, R², R³, R⁴ may represent C(O)R¹² or COOR¹³, where R¹² or R¹³═C₁-C₄ alkyl, optionally substituted with a hydroxyalkyl group (CH₂)_{y}OH, where y=1-6, with a straight or branched alkyl chain, a phosphorylated hydroxyalkyl group (CH₂)ₘOP(O)(OH)₂, where m=1-6, with a straight or branched alkyl chain;
one of the residues R⁵, R⁶ may represent CN;
further, one of the residues R¹, R², R³, R⁴, R⁵, R⁶ may represent (CH₂)_{z}—C₆H₄—NH₂, COC₆H₄—NH₂, CONHC₆H₄—NH₂ or CSNHC₆H₄—NH₂ with z=1-4, and C₆H₄ being a 1,2-, 1,3- or 1,4-phenylene, COC₅H₃N—NH₂ or (CH₂)ₛ—C₅H₃N—NH₂, with z=1-4, and C₅H₃N being pyridin-2,4-diyl, pyridin-2,5-diyl, pyridin-2,6-diyl, or pyridin-3,5-diyl;
additionally, R¹-R² (R³-R⁴, R⁵-R⁶) may form a four-, five, six-, or seven-membered heterocycle comprising the nitrogen atom; which heterocycle may be unsubstituted or substituted with a straight or branched (C₂-C₆) alkyl group, which is unsubstituted, substituted with 1 to 3 halogen atoms or incorporates 1 to 3 heteroatoms selected from O, S, and N, or which is optionally substituted with solubilizing groups selected from OH, NH₂, SH, and COOH and/or ionizable anionic residues selected from (alkyl) carboxy (CH₂)ₙCO₂H, (alkyl) sulfonate (CH₂)ₙSO₃H, (alkyl) sulfate (CH₂)ₙOSO₃H, (alkyl) phosphate (CH₂)ₙOP(O)(OH)₂ and (alkyl) phosphonate (CH₂)ₙP(O)(OH)₂, wherein n is an integer ranging from 0 to 6, or analogs thereof wherein one or more of the CH₂ groups are replaced by CF₂ and/or CD₂;
or R¹-R² (R³-R⁴, R⁵-R⁶) may form a four-, five, six-, or seven-membered cycle (comprising the nitrogen atom) with a hydroxyl group OH, or a phosphorylated hydroxyl group OP(O)(OH)₂ attached to one of the carbon atoms in this cycle, a primary amino group NH₂, secondary amino group NHR^{a} (where R^{a}═C₁-C₆ alkyl of functionally substituted alkyl group), or the combination of such groups;
with the proviso that in all compounds of Formula A 1, 2, 3, 4, 5 or 6 negatively charged groups are present under basic conditions, i.e. 7<pH<14, and these negatively charged groups represent at least partially deprotonated residues of ionizable groups selected from the following set: OH, SH, COOH, a sulfonic acid residue SO₃H, a sulfate residue OSO₃H, an N-cyanosulfonamide residue SO₂NHCN, a phosphate group OP(O)(OH)₂, and a phosphonate group P(O)(OH)₂; 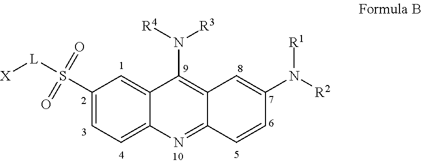
wherein
R¹, R², R³, R⁴ are independent from each other and may represent:
a) hydrogen;
b) CH₃, C₂H₅, a straight or branched C₃-C₆ alkyl, deuterated alkyl, or perfluoroalkyl group;
c) straight or branched C₂-C₆ alkyl group that is substituted with 1 to 3 halogen atoms, or incorporates 1 to 3 heteroatoms selected from O, S, and N, or which is optionally substituted with solubilizing groups selected from OH, NH₂, SH, and COOH and/or ionizable anionic residues selected from (alkyl) carboxy (CH₂)ₙCO₂H, (alkyl) sulfonate (CH₂)ₙSO₃H, (alkyl) sulfate (CH₂)ₙOSO₃H, (alkyl) phosphate (CH₂)ₙOP(O)(OH)₂ and (alkyl) phosphonate (CH₂)ₙP(O)(OH)₂, wherein n is an integer ranging from 0 to 6, or analogs thereof wherein one or more of the CH₂ groups are replaced by CF₂ and/or CD₂ residues;
d) (CH₂)ₙCOOR⁷, where n=1-6, and R⁷ is alkyl, CH₂CN, CH₂CF₃, benzyl, substituted benzyl, polyhaloalkyl, polyhalophenyl, 2- and 4-nitrophenyl, N-succinimidyl, sulfo-N-succinimidyl, 1-benzotriazolyl, 9-aza-1-benzotriazolyl or other groups forming an active ester of a carboxylic acid;
e) a carbonate or carbamate derivative (CH₂)ₘOCOOR⁸ or (CH₂)ₘNHCOOR⁸, where m=1-6 and R⁸═C₁-C₄ alkyl, benzyl, 9-fluorenylmethyl, CH₂CN, N-succinimidyl, sulfo-N-succinimidyl, 1-benzotriazolyl, phenyl, substituted phenyl group, 2-pyridyl, 4-pyridyl, or pyrimid-4-yl;
f) (CH₂)ₘNR^{a}R^{b}, where m=1-6, with a straight or branched alkyl chain; R^{a}, R^{b} are independent from each other and represent hydrogen, C₁-C₄ alkyl and/or C₁-C₄ acyl groups, optionally substituted with a hydroxyalkyl group (CH₂)ₙOH, where n=1-6, with a straight or branched alkyl chain, a phosphorylated hydroxyalkyl group (CH₂)ₚOP(O)(OH)₂, where p=1-6, with a straight or branched alkyl chain;
g) an alkyl azide (CH₂)_{q}N₃, where q=1-8, with a straight or branched alkyl chain;
h) an alkylalkyne (CH₂)ᵣ≡CR⁹, where r=1-8, with a straight or branched alkyl chain in (CH₂)ᵣ and R⁹=hydrogen, alkyl C₁-C₆ or (functionally) substituted alkyl C₁-C₆;
i) (CH₂)ₛCONHR¹⁰, with s=1-8, R¹⁰═H, C₁-C₆ alkyl, (CH₂)_{q}N₃, (CH₂)ᵣC≡CH or (CH₂)ₜ—N-maleimido, (CH₂)ᵤ—NH—COCH₂X (X ═Br or I), where q, r, t, u indices are independent in the range 1-8, and with straight or branched alkyl chains in (CH₂)ₛ, (CH₂)_{q}, (CH₂)ᵣ, (CH₂)ₜ, (CH₂)ᵤ and R¹⁰;
j) a primary amino group (NH₂) or secondary amino group (NHR¹¹); R¹¹═C₁-C₆ alkyl (functionally) substituted a C₁-C₆ alkyl;
R¹, R², R³, R⁴ may contain a terminal alkyloxyamino group (CH₂)ᵥONH₂, where v=1-12, with a straight or branched alkyl chain that can include one or multiple (alkyl)oxy (CH₂)ₖO, (alkyl)amino (CH₂)ₗNH or (alkyl)amido (CH₂)_{w}CONH groups in all possible combinations with indices k, 1 and w=0-12 independent from each other;
R¹, R², R³, R⁴ may contain a terminal acylhydrazino group (CH₂)ₓCONHNH₂, where x=0-12, with a straight or branched alkyl chain that can include one or multiple (alkyl)oxy (CH₂)ₖO, (alkyl)amino (CH₂)ₗNH or (alkyl)amido (CH₂)nCONH groups in all possible combinations with indices k, 1 and w=0-12 (independent from each other;
one of the residues R¹, R², R³, R⁴ may represent C(O)R¹² or COOR¹³, where R¹² or R¹³═C₁-C₄ alkyl, optionally substituted with a hydroxyalkyl group (CH₂)_{y}OH, where y=1-6, with a straight or branched alkyl chain, a phosphorylated hydroxyalkyl group (CH₂)ₘOP(O)(OH)₂, where m=1-6, with a straight or branched alkyl chain;
further, one of the residues R¹, R², R³, R⁴ may represent (CH₂)_{z}—C₆H₄—NH₂, COC₆H₄—NH₂, CONHC₆H₄—NH₂ or CSNHC₆H₄—NH₂ with z=1-4, and C₆H₄ being a 1,2-, 1,3- or 1,4-phenylene, COC₅H₃N—NH₂ or (CH₂)ₛ—C₅H₃N—NH₂, with z=1-4, and C₅H₃N being pyridin-2,4-diyl, pyridin-2,5-diyl, pyridin-2,6-diyl, or pyridin-3,5-diyl;
additionally, R¹-R² (R³-R⁴) may form a four-, five, six-, or seven-membered cycle (comprising the nitrogen atom); which heterocycle may be unsubstituted or substituted with a straight or branched C₂-C₆ alkyl group, which is unsubstituted, substituted with 1 to 3 halogen atoms or incorporates 1 to 3 heteroatoms selected from O, S, and N, or which is optionally substituted with solubilizing groups selected from OH, NH₂, SH, and COOH) and/or ionizable anionic residues selected from (alkyl) carboxy (CH₂)ₙCO₂H, (alkyl) sulfonate (CH₂)ₙSO₃H, (alkyl) sulfate (CH₂)ₙOSO₃H, (alkyl) phosphate (CH₂)nOP(O)(OH)₂ and (alkyl) phosphonate (CH₂)ₙP(O)(OH)₂, wherein n is an integer ranging from 0 zero to 6, or analogs thereof wherein one or more of the CH₂ groups are replaced by CF₂ and/or CD₂;
or R¹-R² (R³-R⁴) may form a four-, five, six-, or seven-membered cycle (comprising the nitrogen atom) with a hydroxyl group OH, or a phosphorylated hydroxyl group OP(O)(OH)₂ attached to one of the carbon atoms in this cycle, a primary amino group NH₂, secondary amino group NHR^{a} (where R^{a}═C₁-C₆ alkyl of functionally substituted alkyl group), or the combination of such groups;
with the proviso that in all compounds of Formula A 1 2, 3, 4, 5 or 6 negatively charged groups are present under basic conditions, i.e. 7<pH<14, and these negatively charged groups represent at least partially deprotonated residues of ionizable groups selected from the following set: OH, SH, COOH, a sulfonic acid residue SO₃H, a sulfate residue OSO₃H, an N-cyanosulfonamide residue SO₂NHCN, a phosphate group OP(O)(OH)₂, and a phosphonate group P(O)(OH)₂;
the linker L (connecting the dye core with the group X) comprises at least one carbon atom and can be represented by alkyl, alkyloxy, CH₂OCH₂, CH₂CH₂O, CH₂CH₂OCH₂, difluoromethyl (CF₂), alkene or alkyne moieties in any combinations, at any occurrence, linear or branched, with the length ranging from C₁ to C₁₂, which allow further connection to a solubilizing and/or ionizable moieties X, or incorporation of heteroatoms selected from O, N, and S; into such (a) linker(s);
in particular, the linker L may consist of alkanediyl (C₂-C₁₂), heterosubstituted alkanediyl containing heteroatoms selected from O, N and/or S in any combination, or alkyloxyalkyl (CH₂)ₙO(CH₂)ₘ, alkylthiaalkyl (CH₂)ₙS(CH₂)ₘ, or alkylazaalkyl (CH₂)ₙNR(CH₂)ₘ, where n=1-6, m=1-6 (independent from each other) and R═H or (functionally substituted) alkyl (C₁-C₄);
further on, the linker L can be represented by a divalent residue derived from dialkylamine, diethanolamine or N-alkylmonoethanolamine moieties such as —N(CH₃)CH₂CH₂O- and N(CH₂CH₂O-)₂, difluoromethyl (CF₂), alkene or alkyne moieties in any combinations, at any occurrence, linear or branched, with the number of C-atoms ranging from C₁ to C₁₂;
additionally, the linker L may also include a carbonyl (>CO) moiety, the group CF₂CO, one or several CD₂ groups and/or terminal CD₃ groups;
wherein X denotes a solubilizing and/or ionizable anion-providing moiety selected from hydroxyalkyl (CH₂)ₙOH, thioalkyl (CH₂)ₙSH, carboxy alkyl (CH₂)ₙCO₂H, alkyl sulfonate (CH₂)ₙSO₃H, alkyl sulfate (CH₂)ₙOSO₃H, alkyl phosphate (CH₂)ₙOP(O)(OH)₂ or phosphonate (CH₂)ₙP(O)(OH)₂, wherein n is an integer ranging from 0 to 12 or 1 to 12, or an analog thereof wherein one or more of the CH₂ groups are replaced by CF₂ and/or CD₂;
with the proviso that in all compounds of Formula B 1 2, 3, 4, 5 or 6 negatively charged groups are present under basic conditions, i.e. 7<pH<14, and these negatively charged groups represent at least partially deprotonated residues of ionizable groups selected from hydroxyl OH, thiol SH, carboxyl COOH, a sulfonic acid residue SO₃H, a sulfate residue OSO₃H, an N-cyanosulfonyl group SO₂NHCN, a phosphate group OP(O)(OH)₂, and a phosphonate group P(O)(OH)₂; 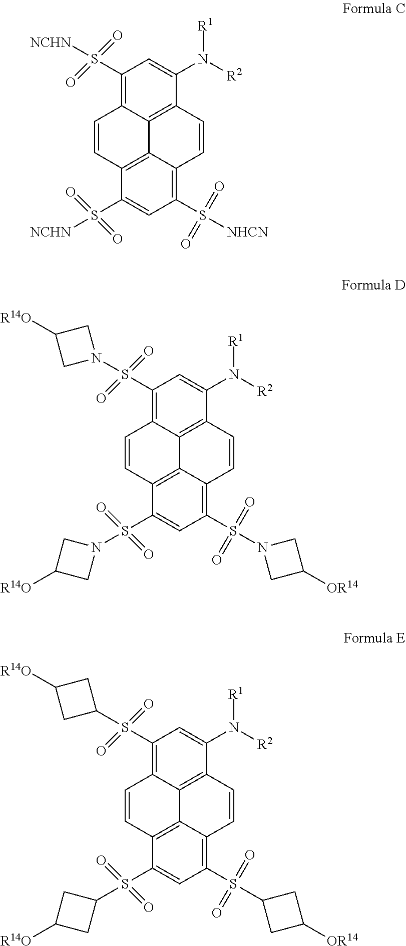
wherein
R¹, R² are independent from each other and selected from the group consisting of
a) hydrogen;
b) CH₃, C₂Hs, a straight or branched C₃-C₆, alkyl, deuterated alkyl, or perfluoroalkyl group;
c) straight or branched C₂-C₆ alkyl group that is unsubstituted, substituted with 1 to 3 halogen atoms, or incorporates 1 to 3 heteroatoms selected from O, S, and N, or optionally substituted with solubilizing groups selected from OH, NH₂, SH, and COOH and/or ionizable anionic residues selected from the group consisting of (alkyl) carboxy (CH₂)ₙCO₂H, (alkyl) sulfonate (CH₂)ₙSO₃H, (alkyl) sulfate (CH₂)ₙOSO₃H, (alkyl) phosphate (CH₂)ₙOP(O)(OH)₂ and (alkyl) phosphonate (CH₂)ₙP(O)(OH)₂, wherein n is an integer ranging from 0 to 6, or analogs thereof wherein one or more of the CH₂ groups are replaced by CF₂ and/or CD₂ residues;
d) (CH₂)ₙCOOR⁷, where n=1-6, and R⁷ may be alkyl, CH₂CN, CH₂CF₃, benzyl, substituted benzyl, CH₂CF₃, polyhaloalkyl, polyhalophenyl, 2- and 4-nitrophenyl, N-succinimidyl, sulfo-N-succinimidyl, 1-benzotriazolyl, 9-aza-1-benzotriazolyl or other groups forming an active ester of a carboxylic acid;
e) a carbonate or carbamate derivative (CH₂)ₘOCOOR⁸ or (CH₂)ₘNHCOOR⁸, where m=1-6 and R⁸═C₁-C alkyl, benzyl, 9-fluorenylmethyl, CH₂CN, N-succinimidyl, sulfo-N-succinimidyl, 1-benzotriazolyl, phenyl, substituted phenyl group, 2-pyridyl, 4-pyridyl, or pyrimid-4-yl;
f) (CH₂)ₘNR^{c}R^{d}, where m=1-6, with a straight or branched alkyl chain; R^{c}, R^{d} are independent from each other and represent hydrogen and/or C₁-C₄ alkyl groups, a hydroxyalkyl group (CH₂)ₘOH, where m=2-6, with a straight or branched alkyl chain, a phosphorylated hydroxyalkyl group (CH₂)ₘOP(O)(OH)₂, where m=2-6, with a straight or branched alkyl chain;
g) an alkyl azide (CH₂)ₘN₃, where m=1-8, with a straight or branched alkyl chain;
h) an alkylalkyne (CH₂)C≡CR⁹, where r=1-8, with a straight or branched alkyl chain in (CH₂)ᵣ and R⁹=hydrogen, alkyl C₁-C₆ or (functionally) substituted alkyl C₁-C₆.
i) (CH₂)ₛCONHR¹⁰, with s=1-8, R¹⁰═H, C₁-C₆ alkyl, (CH₂)_{q}N₃, (CH₂)ᵣC≡CH or (CH₂)ᵣN-maleimido, (CH₂)ₙ—NH—COCH₂X with X═Br or I, where q, r, t, u indices are independent in the range 1-8, and with straight or branched alkyl chains in (CH₂)s, (CH₂)_{q}, (CH₂)ᵣ, (CH₂)t, (CH₂)ᵤ and R¹⁰.
j) a primary amino group (NH₂) or secondary amino group (NHR¹¹); R¹¹=alkyl C₁-C₆ or (functionally) substituted alkyl C₁-C₆;
R¹, R² may contain a terminal alkyloxyamino group (CH₂)ᵥONH₂, where v=1-12, with a straight or branched alkyl chain that can include one or multiple (alkyl)oxy (CH₂)ₖO, (alkyl)amino (CH₂)ₗNH or (alkyl)amido (CH₂)_{w}CONH groups in all possible combinations with indices k, 1 and w=0-12 independent from each other); R¹, R² may contain a terminal acylhydrazino group (CH₂)ₓCONHNH₂, where x=1-12, with a straight or branched alkyl chain that can include one or multiple (alkyl)oxy (CH₂)ₖO, (alkyl)amino (CH₂)ₗNH or (alkyl)amido (CH₂)_{w}CONH groups in all possible combinations with indices k, 1 and w=0-12 independent from each other; one of the residues R¹, R² or both residues R¹ and R² may represent C(O)R¹² or COOR¹³, where R¹² or R¹³═C₁-C₄ alkyl, optionally substituted with a hydroxyalkyl group (CH₂)_{y}OH, where y=1-6, with a straight or branched alkyl chain, a phosphorylated hydroxyalkyl group (CH₂)ₘOP(O)(OH)₂, where m=1-6, with a straight or branched alkyl chain;
further, one of the residues R¹, R² may represent (CH₂)_{z}—C₆H₄—NH₂, COC₆H₄—NH₂, CONHC₆H₄—NH₂ or CSNHC₆H₄—NH₂ with z=1-4, and C₆H₄ being a 1,2-, 1,3- or 1,4-phenylene, COC₅H₃N—NH₂ or (CH₂)S-C₅H₃N—NH₂, with z=1-4, and C₅H₃N being pyridin-2,4-diyl, pyridin-2,5-diyl, pyridin-2,6-diyl, or pyridin-3,5-diyl;
additionally, R¹-R² may form a four-, five, six-, or seven-membered cycle (comprising the nitrogen atom); which (hetero)cycle may be unsubstituted or substituted with a straight or branched C₂-C₆ alkyl group, which is unsubstituted, substituted with 1 to 3 halogen atoms or incorporates 1 to 3 heteroatoms selected from O, S, and N, or which is optionally substituted with solubilizing groups selected from OH, NH₂, SH, and COOH and/or ionizable anionic residues, selected from (alkyl) carboxy (CH₂)ₙCO₂H, (alkyl) sulfonate (CH₂)ₙSO₃H, (alkyl) sulfate (CH₂)ₙOSO₃H, (alkyl) phosphate (CH₂)ₙOP(O)(OH)2 and (alkyl) phosphonate (CH₂)ₙP(O)(OH)₂, wherein n is an integer ranging from 0 to 6, or analogs thereof wherein one or more of the CH₂ groups are replaced by CF₂ and/or CD₂; or R¹-R² may form a four-, five, six-, or seven-membered cycle (comprising the nitrogen atom) decorated with a hydroxyl group OH, or a phosphorylated hydroxyl group OP(O)(OH)₂ attached to one of the carbon atoms in this cycle, a primary amino group NH₂, secondary amino group NHR³ (where R^{a}═C₁-C₆ alkyl of functionally substituted alkyl group), or the combination of such groups;
with the proviso that in all compounds of Formulae C, D, E at least 3 negatively charged groups are present under basic conditions, i.e. 7<pH<14, and these negatively charged groups represent at least partially deprotonated residues of ionizable groups selected from the following set: OH, SH, COOH, a sulfonic acid residue SO₃H, a sulfate residue OSO₃H, an N-cyanosulfonamide residue SO₂NHCN, a phosphate group OP(O)(OH)₂, and a phosphonate group P(O)(OH)₂;
R¹⁴═H or primary phosphate group (P(O)(OH)₂).

2. The fluorescent dye according to claim 1, wherein the analyte-reactive group at variable positions R¹, R² R³, R⁴, R⁵, R⁶ in Formula A or the analyte-reactive group at variable positions R¹, R², R³, R⁴ in Formula B may be represented by an aromatic or heterocyclic amine, carboxylic acid, ester of the carboxylic acid; or represented by alkyl azide (CH₂)ₙN₃, alkyne, strained cyclic alkene, strained cyclic alkyne, amino (NH₂), amino-oxyalkyl (CH₂)ₘONH₂, maleimido or halogeno ketone function COCH₂X with X selected from ═Cl, Br and I, as well as halogeno amide group NRCOCH₂X, with R═H, C₁-C₆-alkyl, X═Cl, Br, I) connected either directly or indirectly via alkandiyl —(CH₂)ₚ—, carbonyl (>CO), amido (—CONR^{a}—), nitrogen (—NR^{b}—), oxygen (—O—) or sulfur-containing linkers (—S—, >SO, >SO₂)n R, R^{a}, R^{b} are independent from each other and represent H, C₁-C₆ alkyl group, or (functionally substituted) C₁-C₆ alkyl group; n, m, p=1-8 (independent from each other).

3. The fluorescent dye according to claim 1, wherein the aryl amino groups (NR¹R² and/or NR³R⁴) in Formula A or B are connected to an analyte-reactive group via (poly)methylene, poly(ethylene glycol), carbonyl, nitrogen or sulfur-containing linear or branched linkers their combinations, or linked as a part of nitrogen-containing non-aromatic heterocycles or alternatively
the aryl amino groups (NR¹R² and/or NR³R⁴) in Formula A or B are connected to an acyl hydrazine or alkyl hydrazine moiety indirectly, via linkers, thus comprising hydrazides (ZCONHNH₂) or hydrazines (ZNHNH₂), respectively, wherein Z denotes the dye residue of Formula A or B that includes aryl amino groups and linkers;
R¹ and R³ may be represented by: (CH₂)ₘCONR, CO(CH₂)ₙNR (R═H, low (substituted) alkyl), CO(CH₂)ₚS(CH₂)ₙ, (CH₂)_{q}S(CH₂)ᵣCO, CO(CH₂)ₛSO₂(CH₂)ₜ, (CH₂)ᵤSO₂(CH₂)_{w}CO and their combinations; m, n, p, q, r, s, t, u, w are independent integers ranging from 0 to 12;
linkers may also be represented by non-aromatic O, N and S-containing heterocycles.

4. The fluorescent dye according to claim 1 of one of general Formulae C-E wherein
R¹, R² are independent from each other and selected from the group consisting of
a) hydrogen;
b) CH₃, C₂H₅, a straight or branched C₃-C₆ group;
c) aminooxyalkyl H₂NO(CH₂)ₙ, wherein n is an integer ranging from 2 to 6, or analogs thereof wherein one or more of the CH₂ groups are replaced by CF₂ and/or CD₂ residues;
d) (CH₂)ₘCOOR⁷, where m=1-6, and R₇ may be H, CH₂CN, CH₂CF₃, CF₃, polyhaloalkyl, polyhalophenyl, e.g. tetra- or pentafluorophenyl, pentachlorophenyl, 2- and 4-nitrophenyl, N-succinimidyl, sulfo-N-succinimidyl, 1-benzotriazolyl, 9-aza-1-benzotriazolyl or other groups forming an active ester of a carboxylic acid;
e) a carbonate or carbamate derivative (CH₂)ₘOCOOR⁸ or (CH₂)ₘNHCOOR⁸, where m=1-6 and R⁸═CH₂CN, N-succinimidyl, sulfo-N-succinimidyl, 1-benzotriazolyl, phenyl, substituted phenyl group, e.g., 2- or 4-nitrophenyl, pentachlorophenyl, pentafluorophenyl, 2,3,5,6-tetrafluorophenyl, 2-pyridyl, 4-pyridyl, pyrimid-4-yl;
f) an alkyl azide (CH₂)ₘN₃, where m=1-8, with a straight or branched alkyl chain; and
g) a terminal acylhydrazino group (CH₂)ₓCONHNH₂, where x=1-12,
with the proviso that in all compounds of Formulae C, D, E at least 3 negatively charged groups are present under basic conditions, i.e. 7<pH<14, and these negatively charged groups represent at least partially deprotonated residues of ionizable groups selected from the following set: OH, SH, COOH, a sulfonic acid residue SO₃H, a sulfate residue OSO₃H, an N-cyanosulfonamide residue SO₂NHCN, a phosphate group OP(O)(OH)₂, and phosphonate group P(O)(OH)₂;
R¹⁴═H or primary phosphate group (P(O)(OH)₂).

5. The fluorescent dye according to claim 1 of Formulae A or B above which has a negative net charge q of −1, −3, −5, or higher; or of Formula C which has a net charge z of −3, or of Formulae D or E above which has a net charge z of 0, −3, or −6, in an aqueous medium at pH ranging from 7 to 13.

6. The fluorescent dye according claim 1 of general Formula A above having one of the following formulae, including salts or protonated forms thereof:

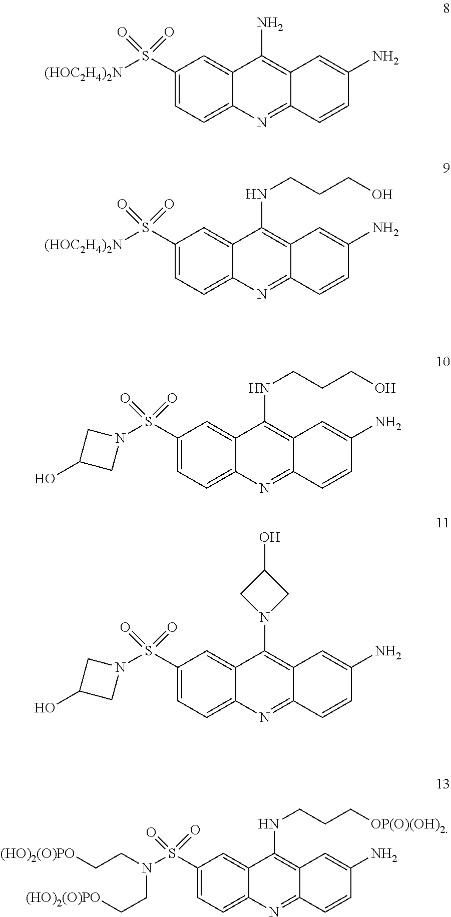

7. The fluorescent dye according claim 1 of general Formula B having one of the following formulae, including salts or protonated forms thereof:

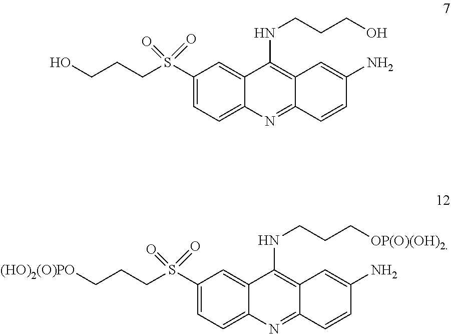

8. The fluorescent dye according claim 1 of one of general Formulae C-E having one of the following formulae, including salts or protonated forms thereof:

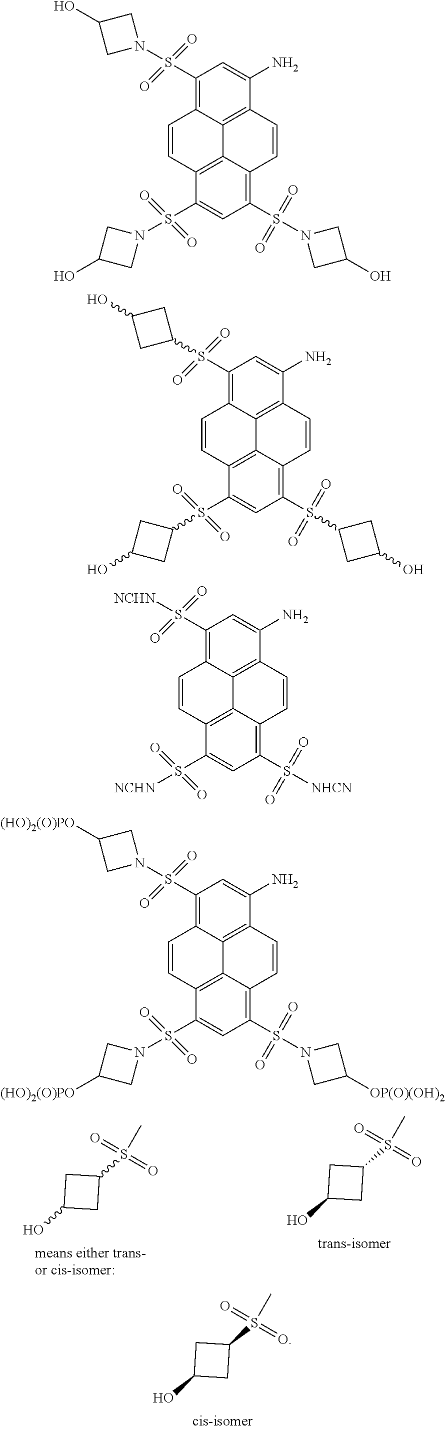

9. The fluorescent dye salt according to claim 1, comprising negatively charged N-cyanamidosulfonate and/or phosphate groups and counterions selected from inorganic or organic cations, and/or comprising a positively charged group or a charge-transfer complex formed at the nitrogen site N(R¹)R² (with R¹ and R² as defined in claim 1 above) in the dye of formulae A-E with a counterion selected from anions of a strong mineral acid, an organic acid or a Lewis acid.

10. A fluorescent dye or dye salt according to claim 1, wherein carbohydrate-reactive groups selected from the group consisting of hydrazine —N(R)NH₂, hydroxylamine —N(R)OH and aminooxy —ONH₂ groups with R being H, (C₁-C₆) alkyl, heteroalkyl; n, m=1-6), alkenyl, or (per)fluoroalkyl (C₁-C₆) are connected to the nitrogen site N(R¹)R² in the dye of formulae A or B via all types of linkers (groups R¹, R², R³, R⁴, R⁵ or R⁶ in structures A and B), or linkers L in formula B.

11. A compound according to claim 1 and salts thereof for use in the reductive amination and in the condensation with reducing sugars, which are monomeric, oligomeric or polymeric carbohydrates possessing an aldehyde group in a free form or as hemiacetal, including glycans.

12. A compound according to claim 1 and salts thereof for use as a fluorescent label for natural products, including amino acids, peptides, proteins, including primary and secondary antibodies, single-domain antibodies, avidin, streptavidin and their modifications, aptamers, nucleotides, nucleic acids, toxins, lipids, carbohydrates, including 2-deoxy-2-amino glucose and other 2-deoxy-2-aminoaminopyranosides, glycans, biotin, and other small molecules having molecular masses of less than 1500 Da, and selected from docetaxel, cabazitaxel, larotaxel, aminophalloidin, jasplakinolide and their modifications.

13. A method of using the compounds according to claim 1 and salts thereof as fluorescent reagents for conjugation to analytes, wherein the conjugation comprises formation of at least one covalent chemical bond or at least one molecular complex with a chemical entity or substance selected from the group consisting of an amine, carboxylic acid, aldehyde, alcohol, aromatic compound, heterocycle, dye, amino acid, peptide, protein, carbohydrate, nucleic acid, toxin and lipid, followed by fluorescence detection, which can be used alone or combined with any other detection method, including mass-spectrometric detection.

14. A carbohydrate-dye conjugate comprising fluorescent dyes according to claim 1, wherein the carbohydrate moiety is a reducing glycans.

15. The carbohydrate-dye conjugate according to claim 14 which is selected from the following structures: 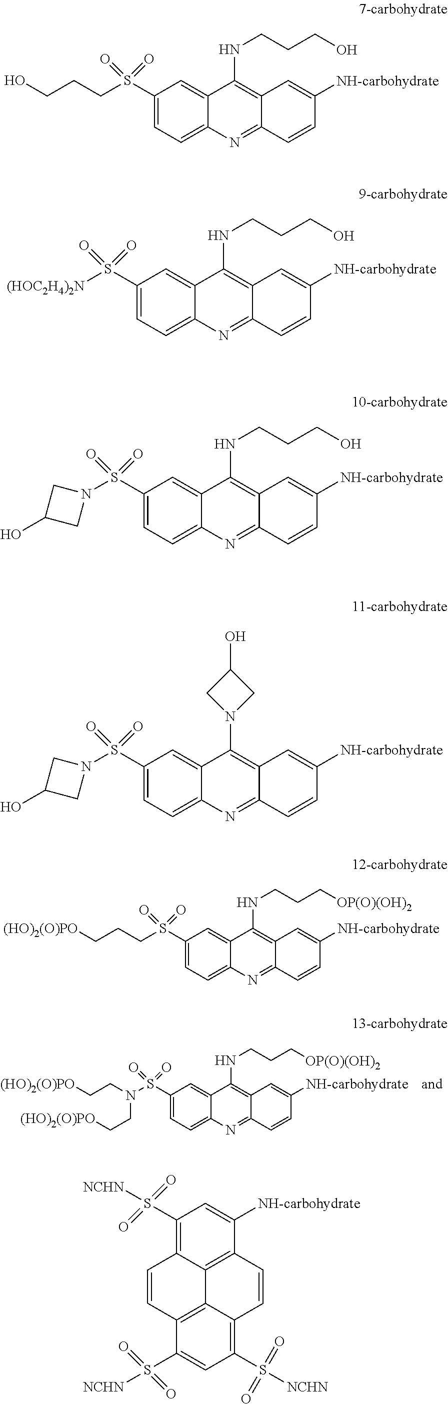 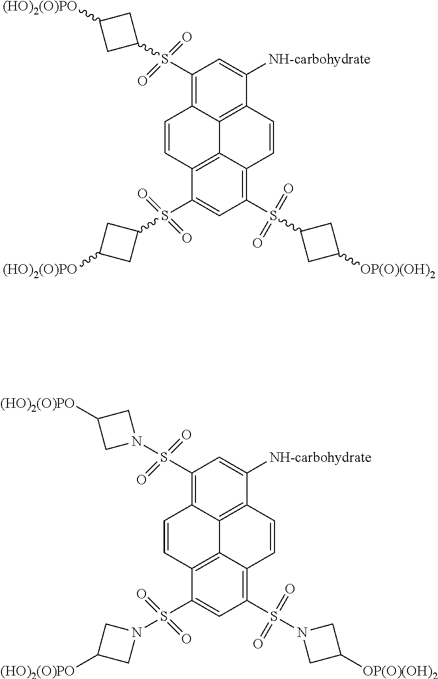
wherein the carbohydrate moiety is a reducing glycan.

16. A kit or composition comprising one or more of the dyes of claim 1.

17. A method for obtaining of dyes' conjugates with reducing sugars based on a two-step procedure: 1) formation of the (protonated) imine intermediate (Schiff's base) prepared from the fluorescent dye, in particular selected from 8-aminopyrene-1,3,6-trisulfonic acid (APTS), the salt thereof, or any dye according to claim 1, and an unlabeled mixture of reducing mono-, di- and oligosaccharides with incremental addition of monomeric units and stepwise increasing molecular masses, and 2) reduction of the said intermediate.

18. The method for obtaining of dyes' conjugates with reducing sugars according to claim 17 (step 1), in which the formation of the protonated imine intermediate (Schiff's base) is facilitated by removal of water and high-boiling solvent from the reaction solution containing: a) APTS, the salt thereof, or the dye, b) a mixture of mixture of reducing mono-, di- and oligosaccharides with incremental addition of monomeric units and stepwise increasing molecular masses; and c) acid with pKa=2-6.

19. The method for obtaining of dyes' conjugates with reducing sugars according to claim 17 (step 1), in which a dye, an individual reducing sugar, or mixture of reducing mono-, di- and oligosaccharides with incremental addition of monomeric units and stepwise increasing molecular masses are dissolved in water, combined with an organic acid dissolved in a suitable solvent, incubated at elevated temperature in a range of 25-70° C. for 0.5-2 h, followed by removal of solvents under reduced pressure.

20. The method according to claim 17, in which a dye is mixed with maltooligosaccharides with incremental addition of monomeric units and stepwise increasing molecular masses, and malonic acid in DMSO, followed by incubation at 25-70° C. for 0.5-2 h, removal of the solvents under reduced pressure, addition of a solution of 2-picoline-borane complex, incubation at 25-70° C. for 1-48 h, and, finally, isolation of the products.

21. A method of characterizing a sample containing one or more glycans, the method comprising:
a) providing a sample containing one or more glycans conjugated with a fluorescent dye “A” as analytes of unknown structure and quantity or concentration and a known quantity of a fluorescent reference glycan standard, wherein the reference glycan standard is labeled with a dye (“B”) according to Formulae A-E of claim 1;
b) contacting the sample with a dye “B” according to Formulae A-E conjugated with known reference glycans that is different from the fluorescent dye “A” conjugated with one or more unknown glycans used in step (a);
c) separating the sample by electrophoresis or HPLC;
d) selective/separate detection of the analytes and reference glycans due to different emission signatures of dyes “A” and “B” and
e) quantifying at least one glycan in the sample relative to the reference glycan standard.

22. A method for spectral calibration of a fluorescence detector which comprises injecting a solution of a dye mixture containing 2 or more fluorescent dyes according to claim 1 into an electrophoresis device followed by separation and detection of light emitted by each component of the dye mixture by a CCD camera, diodes array detector or similar device providing the spectral resolution and separate detection of the emitted light.

23. A method for spectral calibration of a fluorescence detector which comprises injecting a solution containing 2 or more of the carbohydrate-dye conjugate according to claim 14 as a dye mixture into an electrophoresis device followed by separation and detection of light emitted by each component of the dye mixture by a CCD camera, diodes array detector or similar device providing the spectral resolution and separate detection of the emitted light.

24. The carbohydrate-dye conjugate according to claim 14, wherein the glycans are selected from mannose, N-acetylglucosamine and N-acetylgalactosamine residues, galactose, fucose, glucose, maltose and/or their oligomers, as well as sialic acids in various possible combinations.

25. The carbohydrate-dye conjugate according to claim 15, wherein the glycans are selected from mannose, N-acetylglucosamine and N-acetylgalactosamine residues, galactose, fucose, glucose, maltose and/or their oligomers, as well as sialic acids in various possible combinations.

26. The method for spectral calibration of a fluorescence detector according to claim 22, wherein the detector is a detector for detection of laser induced fluorescence (LIF) including a detector used in capillary gel electrophoresis with detection by laser induced fluorescence (CGE-LIF).
